(19) United States
(12) Patent Application Publication
(10) Pub. No.: US 2024/0245792 A1
(43) Pub. Date: Jul. 25, 2024

(54) HEAD AND NECK CANCER COMBINATION THERAPY COMPRISING AN IL-2 CONJUGATE AND PEMBROLIZUMAB

(71) Applicants: Synthorx, Inc., La Jolla, CA (US); MSD INTERNATIONAL GMBH, Luzern (CH); MSD INTERNATIONAL BUSINESS GMBH, Luzern (CH)

(72) Inventors: Giovanni Abbadessa, Winchester, MA (US); Carolina E. Caffaro, San Diego, CA (US); Brigitte Demers, Paris (FR); Joseph Leveque, San Diego, CA (US); Wan-Ju Meng, Cambridge, MA (US); Marcos Milla, Carlsbad, CA (US); Jerod Ptacin, San Diego, CA (US)

(21) Appl. No.: 18/525,282

(22) Filed: Nov. 30, 2023

Related U.S. Application Data
(63) Continuation of application No. PCT/US2022/031968, filed on Jun. 2, 2022.
(60) Provisional application No. 63/196,448, filed on Jun. 3, 2021.
(60) Provisional application No. 63/214,634, filed on Jun. 24, 2021.
(60) Provisional application No. 63/253,892, filed on Oct. 8, 2021.
(60) Provisional application No. 63/257,921, filed on Oct. 20, 2021.

(51) Int. Cl. A61K 47/64; A61K 39/00; A61P 35/04; C07K 16/22; C07K 16/28

(52) U.S. Cl. CPC A61K 47/642; A61P 35/04; C07K 16/22; C07K 16/2818; C07K 16/2863; A61K 2039/507

(57) ABSTRACT

Disclosed herein are methods for treating head and neck squamous cell carcinoma (HNSCC) in a subject in need thereof, comprising administering IL-2 conjugates in combination with one or more additional agents.

### CROSS-REFERENCE TO RELATED APPLICATIONS

This application is a continuation of International Patent Application No.: PCT/US2022/013968, filed Jun. 2, 2022, which claims the benefit of priority to U.S. Provisional Application No. 63/196,448, filed on Jun. 3, 2021, U.S. Provisional Application No. 63/214,634, filed on Jun. 24, 2021, U.S. Provisional Application No. 63/253,892, filed on Oct. 8, 2021, and U.S. Provisional Application No. 63/257,921, filed on Oct. 20, 2021.

### REFERENCE TO A SEQUENCE LISTING

This application contains a Sequence Listing which has been submitted electronically in xml format and is hereby incorporated by reference in its entirety. Said xml copy, created on Mar. 7, 2024, is named SeqList2-052838-00138.xml and is 56,135 bytes in size.

### BACKGROUND OF THE DISCLOSURE

Distinct populations of T cells modulate the immune system to maintain immune homeostasis and tolerance. For example, regulatory T (Treg) cells prevent inappropriate responses by the immune system by preventing pathological self-reactivity while cytotoxic T cells target and destroy infected cells and/or cancerous cells. In some instances, modulation of the different populations of T cells provides an option for treatment of a disease or indication.

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins that modulate the development and differentiation of T and B lymphocytes, cells of the monocytic lineage, neutrophils, basophils, eosinophils, megakaryocytes, and hematopoietic cells. Interleukins are produced by helper CD4+T and B lymphocytes, monocytes, macrophages, endothelial cells, and other tissue residents.

In some instances, interleukin 2 (IL-2) signaling is used to modulate T cell responses and subsequently for treatment of a cancer. Accordingly, in one aspect, provided herein are methods of treating head and neck squamous cell carcinoma (HNSCC) in a subject comprising administering an IL-2 conjugate in combination with one or more additional agents.

PD-1 is recognized as an important molecule in immune regulation and the maintenance of peripheral tolerance. PD-1 is moderately expressed on naive T, B and NKT cells and up-regulated by T/B cell receptor signalling on lymphocytes, monocytes and myeloid cells (Sharpe, Arlene H et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong, Haidong et al., Tumor-associated B7-H1 promotes T-cell apoptosis: a potential mechanism of immune evasion. Nat Med. 2002 August; 8(8):793-800; Yang, Wanhua et al., PD-1 interaction contributes to the functional suppression of T-cell responses to human uveal melanoma cells in vitro. Invest Ophthalmol Vis Sci. 2008 June; 49(6 (2008): 49: 2518-2525; Ghebeh, Hazem et al., The B7-H1 (PD-L1) T lymphocyte-inhibitory molecule is expressed in breast cancer patients with infiltrating ductal carcinoma: correlation with important high-risk prognostic factors. Neoplasia (2006) 8: 190-198; Hamanishi, Junzo et al., Programmed cell death 1 ligand 1 and tumor-infiltrating CD8+T lymphocytes are prognostic factors of human ovarian cancer. Proc. Natl. Acad. Sci. USA (2007): 104: 3360-3365; Thompson, R Houston, and Eugene D Kwon, Significance of B7-H1 overexpression in kidney cancer. Clinical genitourin Cancer (2006): 5: 206-211; Nomi, Takeo et al., Clinical significance and therapeutic potential of the programmed death-1 ligand/programmed death-1 pathway in human pancreatic cancer. Clinical Cancer Research (2007); 13:2151-2157; Ohigashi, Yuichiro et al., Clinical significance of programmed death-1 ligand-1 and programmed death-1 ligand 2 expression in human esophageal cancer. Clin. Cancer Research (2005): 11: 2947-2953; Inman, Brant A et al., PD-L1 (B7-H1) expression by urothelial carcinoma of the bladder and BCG-induced granulomata: associations with localized stage progression. Cancer (2007): 109: 1499-1505; Shimauchi, Takatoshi et al., Augmented expression of programmed death-1 in both neoplasmatic and nonneoplastic CD4+ T-cells in adult T-cell Leukemia/Lymphoma. Int. J. Cancer (2007): 121:2585-2590; Gao, Qiang et al., Overexpression of PD-L1 significantly associates with tumor aggressiveness and postoperative recurrence in human hepatocellular carcinoma. Clinical Cancer Research (2009) 15: 971-979; Nakanishi, Juro et al., Overexpression of B7-H1 (PD-L1) significantly associates with tumor grade and postoperative prognosis in human urothelial cancers. Cancer Immunol Immunother. (2007) 56: 1173-1182; Hino et al., Tumor cell expression of programmed cell death-1 is a prognostic factor for malignant melanoma. Cancer (2010): 00: 1-9). Similarly, PD-1 expression on tumor infiltrating lymphocytes was found to mark dysfunctional T cells in breast cancer and melanoma (Ghebeh, Hazem et al., Foxp3+ tregs and B7-H1+/PD-1+T lymphocytes co-infiltrate the tumor tissues of high-risk breast cancer patients: implication for immunotherapy. BMC Cancer. 2008 Feb. 23; 8:57; Ahmadzadeh, Mojgan et al., Tumor antigen-specific CD8 T cells infiltrating the tumor express high levels of PD-1 and are functionally impaired. Blood (2009) 114: 1537-1544) and to correlate with poor prognosis in renal cancer (Thompson, R Houston et al., PD-1 is expressed by tumor infiltrating cells and is associated with poor outcome for patients with renal carcinoma. Clinical Cancer Research (2007) 15: 1757-1761). Thus, it has been proposed that PD-L1-expressing tumor cells interact with PD-1-expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab (KEYTRUDA®, Merck & Co., Inc., Rahway, NJ, USA) is a potent humanized immunoglobulin G4 (IgG4) mAb with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Keytruda® (pembrolizumab) is indicated for the treatment of patients across a number of indications and is indicated for the first-line treatment of patients with unresectable or metastatic CRC that is microsatellite instability-high or mismatch repair deficient (MSI-H/dMMR). Pembrolizumab is the current standard of care for first line MSI-H/dMMR mCRC.

### SUMMARY OF THE DISCLOSURE

Described herein are methods of treating head and neck squamous cell carcinoma (HNSCC) in a subject in need thereof, comprising administering to the subject an IL-2 conjugate in combination with one or more additional agents, wherein the IL-2 conjugate comprises the amino acid sequence of SEQ ID NO: 1 having an unnatural amino acid residue described herein at position 64, e.g., the amino acid sequence of SEQ ID NO: 2.

The invention relates to methods of treating head and neck squamous cell carcinoma (HNSCC) in a subject in need thereof, comprising administering to the subject an IL-2 conjugate in combination with an amount of PD-1 antagonist, wherein the IL-2 conjugate comprises the amino acid sequence of SEQ ID NO: 1 having an unnatural amino acid residue described herein at position 64, e.g., the amino acid sequence of SEQ ID NO: 2.

The invention relates to methods of treating head and neck squamous cell carcinoma (HNSCC) in a subject in need thereof, comprising administering to the subject an amount of PD-1 antagonist in combination with an amount of an IL-2 conjugate, wherein the IL-2 conjugate comprises the amino acid sequence of SEQ ID NO: 1 having an unnatural amino acid residue described herein at position 64, e.g., the amino acid sequence of SEQ ID NO: 2.

Exemplary embodiments include the following.

Embodiment 1. A method of treating head and neck squamous cell carcinoma (HNSCC) in a subject in need thereof, comprising administering to the subject a combination therapy comprising (a) an IL-2 conjugate and (b) pembrolizumab, wherein:
the subject has recurrent and/or metastatic HNSCC; and
the IL-2 conjugate comprises the amino acid sequence of SEQ ID NO: 1 wherein the amino acid at position P64 is replaced by the structure of Formula (I):

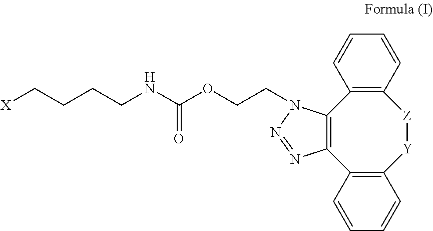

wherein:
Z is CH₂ and Y is

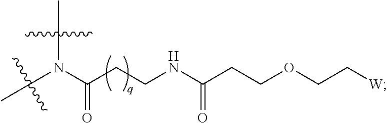

Y is CH₂ and Z is

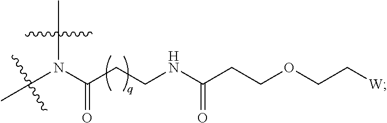

Z is CH₂ and Y is

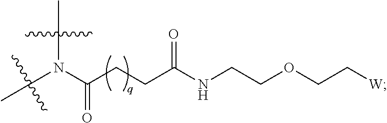

or
Y is CH₂ and Z is

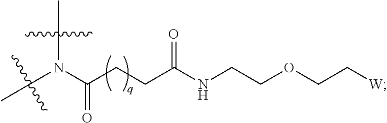

W is a PEG group having an average molecular weight of about 25 kDa-35 kDa;
q is 1, 2, or 3;
X is an L-amino acid having the structure:

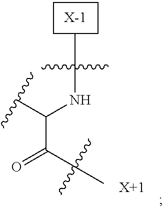 [SMILES: "X-1" ; *NC(*)C(*)=O ; "X+1"]

X−1 indicates the point of attachment to the preceding amino acid residue; and
X+1 indicates the point of attachment to the following amino acid residue.

Embodiment 2. A method of treating head and neck squamous cell carcinoma (HNSCC) in a subject in need thereof, comprising:
selecting a subject having HNSCC, wherein the subject is selected at least in part on the basis of the subject having recurrent and/or metastatic HNSCC; and
administering to the subject a combination therapy comprising (a) an IL-2 conjugate, and (b) pembrolizumab, wherein:
the IL-2 conjugate comprises the amino acid sequence of SEQ ID NO: 1 wherein the amino acid at position P64 is replaced by the structure of Formula (I):

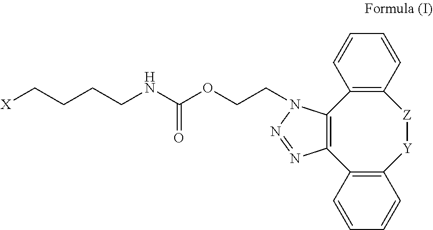

wherein:
Z is CH₂ and Y is

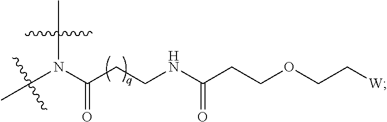

Y is CH₂ and Z is

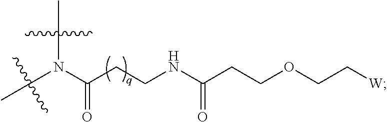

Z is CH₂ and Y is

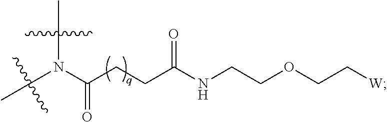

or
Y is CH₂ and Z is

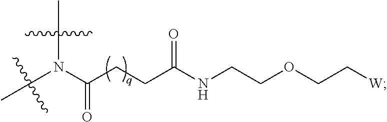

W is a PEG group having an average molecular weight of about 25 kDa-35 kDa;
q is 1, 2, or 3;
X is an L-amino acid having the structure:

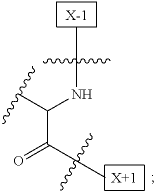

X−1 indicates the point of attachment to the preceding amino acid residue; and
X+1 indicates the point of attachment to the following amino acid residue.

Embodiment 3. The method of embodiment 1 or 2, wherein the method further comprises administering cetuximab to the subject.

Embodiment 4. The method of embodiment 1 or 2, wherein the method further comprises administering an anti-transforming growth factor beta (TGFβ) antibody to the subject.

Embodiment 5. A method of treating head and neck squamous cell carcinoma (HNSCC) in a subject in need thereof, comprising administering to the subject a combination therapy comprising (a) an IL-2 conjugate, (b) pembrolizumab, and (c) cetuximab, wherein: the IL-2 conjugate comprises the amino acid sequence of SEQ ID NO: 1 wherein the amino acid at position P64 is replaced by the structure of Formula (I):

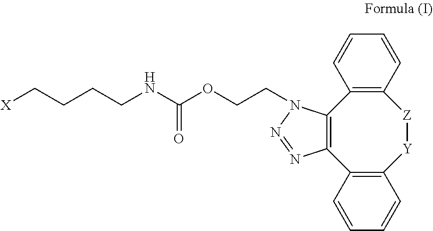

wherein:
Z is CH₂ and Y is

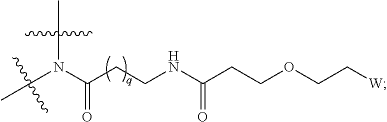

Y is CH₂ and Z is

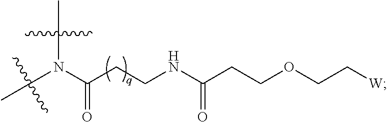

Z is CH₂ and Y is

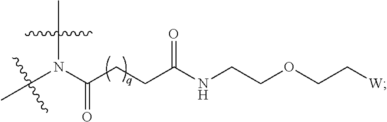

or
Y is CH₂ and Z is

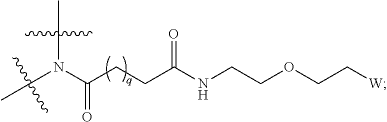

W is a PEG group having an average molecular weight of about 25 kDa-35 kDa;
q is 1, 2, or 3;
X is an L-amino acid having the structure:

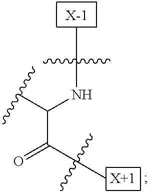

X−1 indicates the point of attachment to the preceding amino acid residue; and
X+1 indicates the point of attachment to the following amino acid residue.

Embodiment 6. A method of treating head and neck squamous cell carcinoma (HNSCC) in a subject in need thereof, comprising administering to the subject a combination therapy comprising (a) an IL-2 conjugate, (b) pembrolizumab, and (c) an anti-transforming growth factor beta (TGFβ) antibody, wherein:
the IL-2 conjugate comprises the amino acid sequence of SEQ ID NO: 1 wherein the amino acid at position P64 is replaced by the structure of Formula (I):

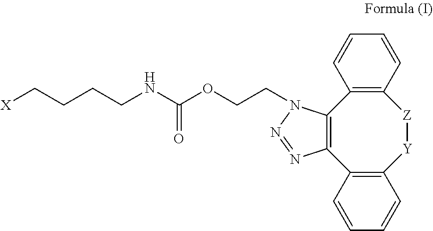

wherein:
Z is CH₂ and Y is

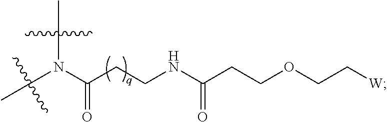

Y is CH₂ and Z is

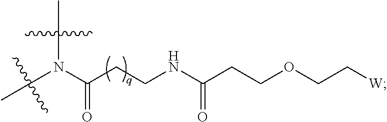

Z is CH₂ and Y is

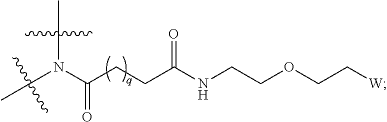

or
Y is CH₂ and Z is

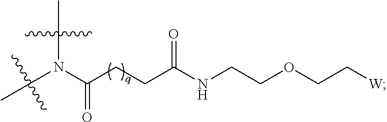

W is a PEG group having an average molecular weight of about 25 kDa-35 kDa;
q is 1, 2, or 3;
X is an L-amino acid having the structure:

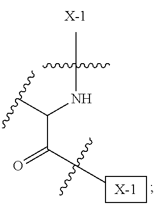 [SMILES: "X-1" ; *NC(*)C(*)=O ; "X-1"]

X−1 indicates the point of attachment to the preceding amino acid residue; and
X+1 indicates the point of attachment to the following amino acid residue.

Embodiment 7. The method of any one of embodiments 1-6, wherein the subject has a PD-L1 combined positive score (CPS) greater than or equal to 1.

Embodiment 8. The method of any one of embodiments 1-7, wherein the subject is treatment-naïve for recurrent and/or metastatic HNSCC.

Embodiment 9. A method of treating head and neck squamous cell carcinoma (HNSCC) in a subject in need thereof, comprising administering to the subject a combination comprising (a) an IL-2 conjugate and (b) pembrolizumab, wherein:
the HNSCC is recurrent and/or metastatic HNSCC; and
the IL-2 conjugate comprises the amino acid sequence of SEQ ID NO: 1 wherein the amino acid at position P64 is replaced by the structure of Formula (I):

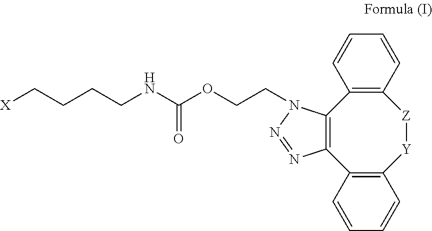

wherein:
Z is CH₂ and Y is

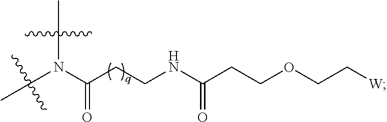

Y is CH₂ and Z is

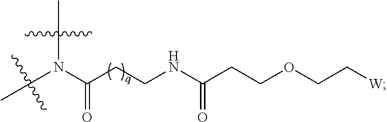

Z is CH₂ and Y is

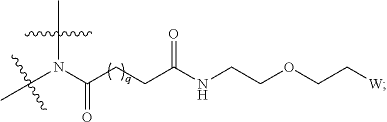

or
Y is CH₂ and Z is

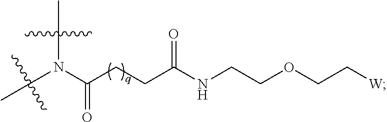

W is a PEG group having an average molecular weight of about 25 kDa-35 kDa;
q is 1, 2, or 3;
X is an L-amino acid having the structure:

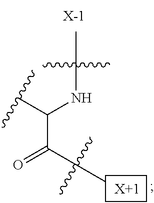 [SMILES: "X-1" ; *NC(*)C(*)=O ; "X+1"]

X−1 indicates the point of attachment to the preceding amino acid residue; and
X+1 indicates the point of attachment to the following amino acid residue.

Embodiment 10. A method of treating head and neck squamous cell carcinoma (HNSCC) in a subject in need thereof, comprising:
selecting a subject having HNSCC, wherein the subject is selected at least in part on the basis of the subject having recurrent and/or metastatic HNSCC; and
administering to the subject a combination comprising (a) an IL-2 conjugate, and (b) pembrolizumab, wherein:
the IL-2 conjugate comprises the amino acid sequence of SEQ ID NO: 1 wherein the amino acid at position P64 is replaced by the structure of Formula (I):

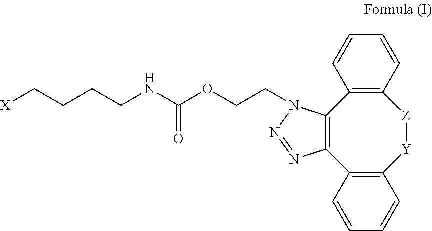

wherein:
Z is CH₂ and Y is

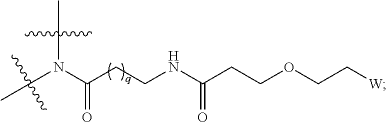

Y is CH₂ and Z is

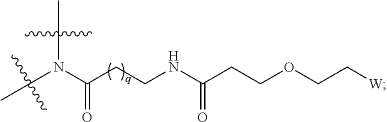

Z is CH₂ and Y is

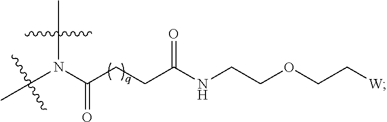

or
Y is CH₂ and Z is

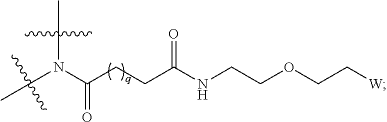

W is a PEG group having an average molecular weight of about 25 kDa-35 kDa;
q is 1, 2, or 3;
X is an L-amino acid having the structure:

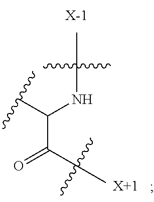

X−1 indicates the point of attachment to the preceding amino acid residue; and
X+1 indicates the point of attachment to the following amino acid residue.

Embodiment 11. The method of embodiment 9 or 10, wherein the subject was previously treated with a PD-1/PD-L1-based regimen.

Embodiment 12. The method of any one of embodiments 3, 5, 7, and 8, wherein the subject was not previously treated with cetuximab.

Embodiment 13. The method of any one of embodiments 9-12, wherein the subject has platinum-refractory HNSCC.

Embodiment 14. The method of any one of embodiments 9-13, wherein the subject was previously treated for HNSCC and the previous treatment for HNSCC comprised failure of no more than two regimens.

Embodiment 15. The method of any one of embodiments 9-14, wherein the subject has platinum-refractory HNSCC and the subject's previous treatment for HNSCC comprised failure of one regimen.

Embodiment 16. The method of any one of embodiments 9-14, wherein the subject has platinum-refractory HNSCC and the subject's previous treatment for HNSCC comprised failure of two regimens.

Embodiment 17. The method of any one of embodiments 1-16, comprising administering to the subject about 8 μg/kg to 32 μg/kg of the IL-2 conjugate.

Embodiment 18. The method of any one of embodiments 1-17, comprising administering to the subject about 8 μg/kg of the IL-2 conjugate.

Embodiment 19. The method of any one of embodiments 1-17, comprising administering to the subject about 16 μg/kg of the IL-2 conjugate.

Embodiment 20. The method of any one of embodiments 1-17, comprising administering to the subject about 24 μg/kg of the IL-2 conjugate.

Embodiment 21. The method of any one of embodiments 1-17, comprising administering to the subject about 32 μg/kg of the IL-2 conjugate.

Embodiment 22. The method of any one of embodiments 1-21, wherein in the IL-2 conjugate the PEG group has an average molecular weight of about 30 kDa.

Embodiment 23. The method of any one of embodiments 1-22, wherein in the IL-2 conjugate Z is CH₂ and Y is

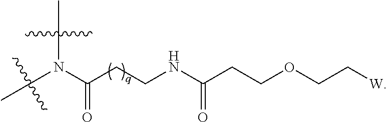

Embodiment 24. The method of any one of embodiments 1-22, wherein in the IL-2 conjugate Y is CH₂ and Z is

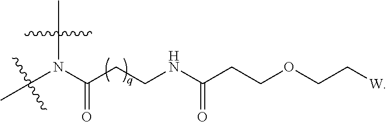

Embodiment 25. The method of any one of embodiments 1-22, wherein in the IL-2 conjugate Z is CH₂ and Y is

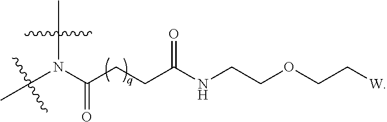

Embodiment 26. The method of any one of embodiments 1-22, wherein in the IL-2 conjugate Y is CH₂ and Z is

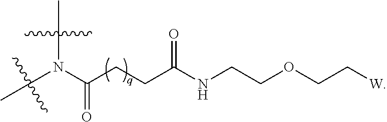

Embodiment 27. The method of any one of embodiments 1-22, wherein the structure of Formula (I) has the structure of Formula (IV) or Formula (V), or is a mixture of Formula (IV) and Formula (V):

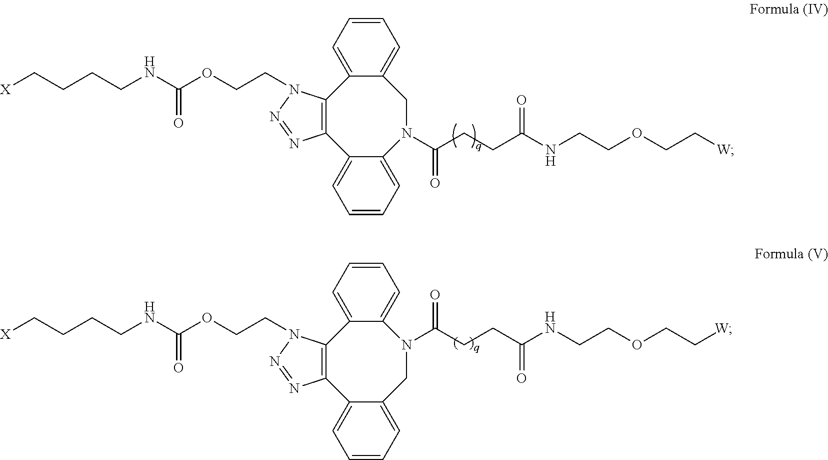

wherein:
W is a PEG group having an average molecular weight of about 25 kDa-35 kDa;
q is 1, 2, or 3;
X is an L-amino acid having the structure:

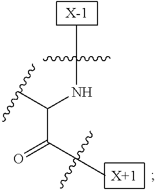

X−1 indicates the point of attachment to the preceding amino acid residue; and
X+1 indicates the point of attachment to the following amino acid residue.

Embodiment 28. The method of any one of embodiments 1-22, wherein the structure of Formula (I) has the structure of Formula (XII) or Formula (XIII), or is a mixture of Formula (XII) and Formula (XIII):

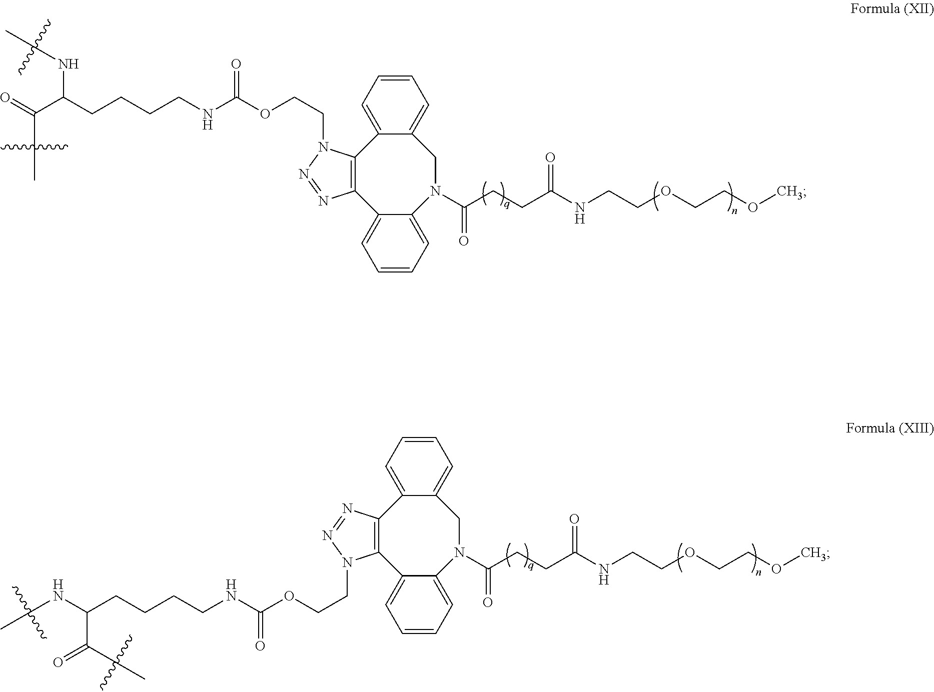

wherein:
n is an integer such that —(OCH₂CH₂)ₙ—OCH₃ has a molecular weight of about 30 kDa;
q is 1, 2, or 3; and
the wavy lines indicate covalent bonds to amino acid residues within SEQ ID NO: 1 that are not replaced.

Embodiment 29. The method of any one of embodiments 1-28, wherein q is 1.

Embodiment 30. The method of any one of embodiments 1-28, wherein q is 2.

Embodiment 31. The method of any one of embodiments 1-28, wherein q is 3.

Embodiment 32. The method of any one of embodiments 1-31, wherein the average molecular weight is a number average molecular weight.

Embodiment 33. The method of any one of embodiments 1-31, wherein the average molecular weight is a weight average molecular weight.

Embodiment 34. The method of any one of embodiments 1-33, wherein the IL-2 conjugate is administered to the subject about once every two weeks, about once every three weeks, or about once every 4 weeks.

Embodiment 35. The method of any one of embodiments 1-33, wherein the IL-2 conjugate and pembrolizumab are administered to the subject about once every two weeks, about once every three weeks, or about once every 4 weeks.

Embodiment 36. The method of any one of embodiments 3, 5, 7, 8, and 12-35, wherein the IL-2 conjugate and cetuximab are administered to the subject about once every two weeks, about once every three weeks, or about once every 4 weeks.

Embodiment 37. The method of any one of embodiments 4, 6, 7, 8, and 17-35, wherein the IL-2 conjugate and the anti-TGFβ antibody are administered to the subject about once every two weeks, about once every three weeks, or about once every 4 weeks.

Embodiment 38. The method of any one of embodiments 1-37, wherein the IL-2 conjugate is a pharmaceutically acceptable salt, solvate, or hydrate.

Embodiment 39. The method of any one of embodiments 1-38, wherein pembrolizumab is administered at a dose of about 200 mg every 3 weeks.

Embodiment 40. The method of any one of embodiments 1-38, wherein pembrolizumab is administered at a dose of about 400 mg every 6 weeks.

Embodiment 41. The method of any one of embodiments 1-38, wherein pembrolizumab is administered at a dose of about 2 mg/kg every 3 weeks.

Embodiment 42. The method of any one of embodiments 1-41, wherein the IL-2 conjugate and pembrolizumab are administered separately.

Embodiment 43. The method of embodiment 42, wherein the IL-2 conjugate and pembrolizumab are administered sequentially.

Embodiment 44. The method of embodiment 43, wherein the IL-2 conjugate is administered after pembrolizumab.

Embodiment 45. The method of embodiment 43, wherein pembrolizumab is administered after the IL-2 conjugate.

Embodiment 46. The method of any one of embodiments 3, 5, 7, 8, 12-36, and 38-45, wherein the initial dose of cetuximab is administered at a dose of about 400 mg/m², and subsequent doses of cetuximab are administered at a dose of about 250 mg/m².

Embodiment 47. The method of any one of embodiments 3, 5, 7, 8, 12-36, and 38-46, wherein cetuximab is administered after pembrolizumab.

Embodiment 48. The method of any one of embodiments 3, 5, 7, 8, 12-36, and 38-47, wherein cetuximab is administered before the IL-2 conjugate.

Embodiment 49. The method of any one of embodiments 3, 5, 7, 8, 12-36, and 38-48, wherein the IL-2 conjugate, pembrolizumab, and cetuximab are administered separately.

Embodiment 50. The method of embodiment 49, wherein the IL-2 conjugate, pembrolizumab, and cetuximab are administered sequentially.

Embodiment 51. The method of any one of embodiments 4, 6, 7, 8, 17-35, and 37-45, wherein the anti-TGFβ antibody is administered after the IL-2 conjugate.

Embodiment 52. The method of any one of embodiments 4, 6, 7, 8, 17-35, 37-45, and 51, wherein the IL-2 conjugate, pembrolizumab, and the anti-TGFβ antibody are administered separately.

Embodiment 53. The method of embodiment 51, wherein the IL-2 conjugate, pembrolizumab, and the anti-TGFβ antibody are administered sequentially.

Embodiment 54. The method of any one of embodiments 3, 5, 7, 8, 12-36, and 38-47, wherein the IL-2 conjugate and cetuximab are administered separately.

Embodiment 55. The method of embodiment 54, wherein the IL-2 conjugate and cetuximab are administered sequentially.

Embodiment 56. The method of embodiment 55, wherein the IL-2 conjugate is administered after cetuximab.

Embodiment 57. The method of any one of embodiments 1-56, wherein the IL-2 conjugate is administered to the subject by subcutaneous administration.

Embodiment 58. The method of any one of embodiments 1-57, wherein the IL-2 conjugate and pembrolizumab are administered to the subject by subcutaneous administration.

Embodiment 59. The method of any one of embodiments 3, 5, 7, 8, 12-36, 38-50, and 54-57, wherein the IL-2 conjugate, pembrolizumab, and cetuximab are administered to the subject by subcutaneous administration.

Embodiment 60. The method of any one of embodiments 4, 6, 7, 8, 17-35, 37-45, and 51-53, wherein the IL-2 conjugate, pembrolizumab, and the anti-TGFβ antibody are administered to the subject by subcutaneous administration.

Embodiment 61. The method of any one of embodiments 3, 5, 7, 8, 12-36, 38-50, and 55-56, wherein the wherein the IL-2 conjugate and cetuximab are administered to the subject by subcutaneous administration.

Embodiment 62. The method of any one of embodiments 1-56, wherein the IL-2 conjugate is administered to the subject by intravenous administration.

Embodiment 63. The method of any one of embodiments 1-56, wherein the IL-2 conjugate and pembrolizumab are administered to the subject by intravenous administration.

Embodiment 64. The method of any one of embodiments 3, 5, 7, 8, 12-36, 38-50, and 54-56, wherein the IL-2 conjugate, pembrolizumab, and cetuximab are administered to the subject by intravenous administration.

Embodiment 65. The method of any one of embodiments 4, 6, 7, 8, 17-35, 37-45, and 51-53, wherein the IL-2 conjugate, pembrolizumab, and the anti-TGFβ antibody are administered to the subject by intravenous administration.

Embodiment 66. The method of any one of embodiments 1-65, further comprising administering acetaminophen to the subject.

Embodiment 67. The method of any one of embodiments 1-66, further comprising administering diphenhydramine to the subject.

Embodiment 68. The method of any one of embodiments 1-67, further comprising administering ondansetron to the subject.

Embodiment 69. The method of any one of embodiments 65-68, wherein the acetaminophen, diphenhydramine, and/or ondansetron is administered to the subject before administering the IL-2 conjugate.

Embodiment 70. The method of any one of embodiments 65-68, wherein the acetaminophen, diphenhydramine, and/or ondansetron is administered to the subject before administering cetuximab.

Embodiment 71. An IL-2 conjugate for use in the method of any one of embodiments 1-70.

Embodiment 72. Use of an IL-2 conjugate for the manufacture of a medicament for the method of any one of embodiments 1-70.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows the change in peripheral CD8+T_{eff} counts in the indicated subjects at specified times following administration of 8 μg/kg IL-2 conjugate and pembrolizumab. Here and elsewhere, designations such as “C1D1” indicate the treatment cycle and day (e.g., treatment cycle 1, day 1). “PRE” indicates the baseline measurement before administration; 24 HR indicates 24 hours after administration; and so on.

FIG. 1B shows the change in peak peripheral CD8+T_{eff} cell expansion following administration of the first dose of IL-2 conjugate and pembrolizumab. Data is normalized to pre-treatment (C1D1) CD8+ T cell count. Listed values indicate median fold changes.

FIG. 1C shows the change in peripheral CD8+T_{eff} counts in the indicated subjects at specified times following administration of 16 μg/kg IL-2 conjugate and pembrolizumab.

FIG. 2 shows the percentage of CD8+T_{eff} cells expressing Ki67 in the indicated subjects at specified times following administration of 8 μg/kg IL-2 conjugate and pembrolizumab.

FIG. 3A shows the change in peripheral natural killer (NK) cell counts in the indicated subjects at specified times following administration of 8 μg/kg IL-2 conjugate and pembrolizumab.

FIG. 3B shows the change in peak peripheral NK cell expansion following administration of the first dose of IL-2 conjugate and pembrolizumab. Data is normalized to pre-treatment (C1D1) NK cell count. Listed values indicate median fold changes.

FIG. 3C shows the change in peripheral natural killer (NK) cell counts in the indicated subjects at specified times following administration of 16 μg/kg IL-2 conjugate and pembrolizumab.

FIG. 4 shows the percentage of NK cells expressing Ki67 in the indicated subjects at specified times following administration of 8 μg/kg IL-2 conjugate and pembrolizumab.

FIG. 5A shows the change in peripheral CD4+T_{reg} counts in the indicated subjects at specified times following administration of 8 μg/kg IL-2 conjugate and pembrolizumab.

FIG. 5B shows the change in peak peripheral CD4+T_{reg} cell expansion following administration of the first dose of IL-2 conjugate and pembrolizumab. Data is normalized to pre-treatment (C1D1) CD4+ T cell count. Listed values indicate median fold changes.

FIG. 5C shows the change in peripheral CD4+T_{reg} counts in the indicated subjects at specified times following administration of 16 μg/kg IL-2 conjugate and pembrolizumab.

FIG. 6 shows the percentage of CD4+T_{reg} cells expressing Ki67 in the indicated subjects at specified times following administration of 8 μg/kg IL-2 conjugate and pembrolizumab.

FIG. 7A shows the change in eosinophil cell counts in the indicated subjects at specified times following administration of 8 μg/kg IL-2 conjugate and pembrolizumab.

FIG. 7B shows the change in peak peripheral eosinophil cell expansion following administration of the first dose of IL-2 conjugate and pembrolizumab. Data is normalized to pre-treatment (C1D1) eosinophil cell count. Listed values indicate median fold changes.

FIG. 7C shows the change in eosinophil cell counts in the indicated subjects at specified times following administration of 16 μg/kg IL-2 conjugate and pembrolizumab.

FIG. 8A shows serum levels of IFN-7, IL-5, and IL-6 in the indicated subjects at specified times following administration of 8 μg/kg IL-2 conjugate and pembrolizumab.

FIG. 8B shows the serum level of IL-5 following administration of 8 μg/kg IL-2 conjugate and pembrolizumab. BLQ=below limit of quantification. Data is plotted as mean (range BLQ to maximum value).

FIG. 8C shows the serum level of IL-6 following administration of 8 μg/kg IL-2 conjugate and pembrolizumab. BLQ=below limit of quantification. Data is plotted as mean (range BLQ to maximum value).

FIG. 8D shows serum levels of IFN-7, IL-5, and IL-6 in the indicated subjects at specified times following administration of 16 μg/kg IL-2 conjugate and pembrolizumab.

FIG. 9A and FIG. 9B show mean concentrations of the IL-2 conjugate, administered at a dose of 8 μg/kg with pembrolizumab, after 1 and 2 cycles, respectively.

FIG. 9C and FIG. 9D show mean concentrations of the IL-2 conjugate, administered at a dose of 16 μg/kg with pembrolizumab, after 1 and 2 cycles, respectively.

FIG. 10 shows the change in peripheral CD8+T_{eff} cell counts in the indicated subjects at specified times following administration of 24 μg/kg IL-2 conjugate and pembrolizumab.

FIG. 11 shows the change in peripheral NK cell counts in the indicated subjects at specified times following administration of 24 μg/kg IL-2 conjugate and pembrolizumab.

FIG. 12 shows the change in peripheral CD4+T_{reg} cell counts in the indicated subjects at specified times following administration of 24 μg/kg IL-2 conjugate and pembrolizumab.

FIG. 13 shows the change in peripheral eosinophil cell counts in the indicated subjects at specified times following administration of 24 μg/kg IL-2 conjugate and pembrolizumab.

FIG. 14A and FIG. 14B show mean concentrations of the IL-2 conjugate, administered at a dose of 24 μg/kg with pembrolizumab, after 1 and 2 cycles, respectively.

FIG. 15 shows the levels of IFN-7, IL-6, and IL-5 in the indicated subjects treated with 24 μg/kg of the IL-2 conjugate and pembrolizumab at specified times following administration of the IL-2 conjugate.

FIG. 16 shows the change in peripheral CD8+T_{eff} cell counts in the indicated subjects at specified times following administration of 32 μg/kg IL-2 conjugate and pembrolizumab.

FIG. 17 shows the peripheral CD4+T_{reg} cell counts in the indicated subjects at specified times following administration of 32 μg/kg IL-2 conjugate and pembrolizumab.

FIG. 18A and FIG. 18B show mean concentrations of the IL-2 conjugate, administered at a dose of 32 μg/kg with pembrolizumab, after 1 and 2 cycles, respectively.

FIG. 19 shows the levels of IFN-γ, IL-6, and IL-5 in the indicated subjects treated with 32 μg/kg of the IL-2 conjugate and pembrolizumab at specified times following administration of the IL-2 conjugate.

FIGS. 20A-C show the % cytotoxicity in CAL27 cells co-cultured with 3 separate donor human PBMCs and varying amounts of an IL-2 conjugate and cetuximab.

FIG. 21A shows the % cytotoxicity in CAL27 cells co-cultured with human PBMCs and varying amounts of an IL-2 conjugate and cetuximab.

FIG. 21B shows the % cytotoxicity in A431 cells co-cultured with human PBMCs and varying amounts of an IL-2 conjugate and cetuximab.

FIG. 22A shows the cytotoxic effect on A431 cells co-cultured with NK92 cells and treated varying amounts of an IL-2 conjugate and cetuximab.

FIG. 22B shows the % cytotoxicity on DLD-1 cells co-cultured with NK92 cells and treated varying amounts of an IL-2 conjugate and cetuximab.

FIG. 22C shows the % cytotoxicity on FaDu cells co-cultured with NK92 cells and treated varying amounts of an IL-2 conjugate and cetuximab.

FIG. 22D shows the % cytotoxicity on CAL27 cells co-cultured with NK92 cells and treated varying amounts of an IL-2 conjugate and cetuximab.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless the context requires otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, the terms “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “unnatural amino acid” refers to an amino acid other than one of the 20 naturally occurring amino acids. Exemplary unnatural amino acids are described in Young et al., “Beyond the canonical 20 amino acids: expanding the genetic lexicon,” J. of Biological Chemistry 285(15): 11039-11044 (2010), the disclosure of which is incorporated herein by reference.

The term “antibody” herein is used in the broadest sense and encompasses various antibody structures, including but not limited to monoclonal antibodies (including full length monoclonal antibodies), polyclonal antibodies, multispecific antibodies (e.g., bispecific antibodies), humanized, fully human antibodies, chimeric antibodies and camelized single domain antibodies and antibody fragments so long as they exhibit the desired antigen-binding activity. In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

An “antibody fragment” or “antigen binding fragment” refers to a molecule other than an intact antibody that comprises a portion of an intact antibody that binds the antigen to which the intact antibody binds. The antibody fragment retains the ability to bind specifically to the antigen bound by the full-length antibody, e.g. fragments that retain one or more CDR regions, e.g. all six CDRs. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

As used herein, “nucleotide” refers to a compound comprising a nucleoside moiety and a phosphate moiety. Exemplary natural nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP). Exemplary natural deoxyribonucleotides, which comprise a deoxyribose as the sugar moiety, include dATP, dTTP, dCTP, dGTP, dADP, dTDP, dCDP, dGDP, dAMP, dTMP, dCMP, and dGMP. Exemplary natural ribonucleotides, which comprise a ribose as the sugar moiety, include ATP, UTP, CTP, GTP, ADP, UDP, CDP, GDP, AMP, UMP, CMP, and GMP.

“CDR” or “CDRs” as used herein means complementarity determining region(s) in a immunoglobulin variable region, defined using the Kabat numbering system, unless otherwise indicated.

As used herein, “base” or “nucleobase” refers to at least the nucleobase portion of a nucleoside or nucleotide (nucleoside and nucleotide encompass the ribo or deoxyribo variants), which may in some cases contain further modifications to the sugar portion of the nucleoside or nucleotide. In some cases, “base” is also used to represent the entire nucleoside or nucleotide (for example, a “base” may be incorporated by a DNA polymerase into DNA, or by an RNA polymerase into RNA). However, the term “base” should not be interpreted as necessarily representing the entire nucleoside or nucleotide unless required by the context. In the chemical structures provided herein of a base or nucleobase, only the base of the nucleoside or nucleotide is shown, with the sugar moiety and, optionally, any phosphate residues omitted for clarity. As used in the chemical structures provided herein of a base or nucleobase, the wavy line represents connection to a nucleoside or nucleotide, in which the sugar portion of the nucleoside or nucleotide may be further modified. In some embodiments, the wavy line represents attachment of the base or nucleobase to the sugar portion, such as a pentose, of the nucleoside or nucleotide. In some embodiments, the pentose is a ribose or a deoxyribose.

In some embodiments, a nucleobase is generally the heterocyclic base portion of a nucleoside. Nucleobases may be naturally occurring, may be modified, may bear no similarity to natural bases, and/or may be synthesized, e.g., by organic synthesis. In certain embodiments, a nucleobase comprises any atom or group of atoms in a nucleoside or nucleotide, where the atom or group of atoms is capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, they are referred to as nucleobases for simplicity. In some embodiments, when referring to a nucleobase, a “(d)” indicates that the nucleobase can be attached to a deoxyribose or a ribose, while “d” without parentheses indicates that the nucleobase is attached to deoxyribose.

As used herein, a “nucleoside” is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

As used herein, “dose-limiting toxicity” (DLT) is defined as an adverse event occurring within Day 1 through Day 29 (inclusive)±1 day of a treatment cycle that was not clearly or incontrovertibly solely related to an extraneous cause and that meets the criteria set forth in Example 2 for DLT.

As used herein, “PD-L1 combined positive score (CPS)” is the number of PD-L1 staining cells (tumor cells, lymphocytes, macrophages) divided by the total number of viable tumor cells in a specimen, multiplied by 100.

As used herein, a “platinum-refractory” cancer is defined as a cancer in which the disease progresses during platinum-based therapy (i.e., patients do not achieve at least stable disease or a partial response to the platinum-based therapy), or the disease relapses within 6 months after the end of the platinum-based treatment.

As used herein, “treatment-naïve” refers to a subject who has never received treatment with a particular therapy. For example, a subject is treatment-naïve for cetuximab if the subject has never received treatment with cetuximab.

As used herein, “cetuximab” refers to the chimeric (mouse/human) anti-EGFR antibody marketed under the brand name “Erbitux” by Eli Lilly and Co.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“PD-1 antagonist” means any chemical compound or biological molecule that blocks binding of PD-L1 expressed on a cancer cell to PD-1 expressed on an immune cell (T cell, B cell or Natural Killer T cell) and in specific embodiments also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and in specific embodiments blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 2. Pembrolizumab has been approved by the U.S. FDA as described in the Prescribing Information for KEYTRUDA™ (Merck & Co., Inc., Rahway, NJ, USA; initial U.S. approval 2014, updated March 2021).

As used herein, a “pembrolizumab variant” or “a variant thereof” pertaining to a pembrolizumab sequence means a monoclonal antibody that comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### IL-2 Combination Therapy

Interleukin 2 (IL-2) is a pleiotropic type-1 cytokine whose structure comprises a 15.5 kDa four α-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and to a lesser extent, by CD8+ cells, Natural Killer (NK) cells, and Natural killer T (NKT) cells, activated dendritic cells (DCs), and mast cells. IL-2 signaling occurs through interaction with specific combinations of IL-2 receptor (IL-2R) subunits, IL-2Rα (also known as CD25), IL-2Rβ (also known as CD122), and IL-2Rγ (also known as CD132). Interaction of IL-2 with the IL-2Rα forms the “low-affinity” IL-2 receptor complex with a K_{d} of about 10⁻⁸ M. Interaction of IL-2 with IL-2Rβ and IL-2Rγ forms the “intermediate-affinity” IL-2 receptor complex with a K_{d} of about 10⁻⁹ M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, or CD4⁺CD25⁺Foxp3⁺ regulatory T (Treg) cells, mediate maintenance of immune homeostasis by suppression of effector cells such as CD4⁺ T cells, CD8⁺ T cells, B cells, NK cells, and NKT cells. In some instances, Treg cells are generated from the thymus (tTreg cells) or are induced from naïve T cells in the periphery (pTreg cells). In some cases, Treg cells are considered as the mediator of peripheral tolerance. Indeed, in one study, transfer of CD25-depleted peripheral CD4⁺ T cells produced a variety of autoimmune diseases in nude mice, whereas cotransfer of CD4+CD25⁺ T cells suppressed the development of autoimmunity (Sakaguchi, et al., “Immunologic self-tolerance maintained by activated T cells expressing IL-2 receptor alpha-chains (CD25),” J. Immunol. 155(3): 1151-1164 (1995), the disclosure of which is incorporated herein by reference). Augmentation of the Treg cell population down-regulates effector T cell proliferation and suppresses autoimmunity and T cell anti-tumor responses.

IL-2 signaling via the “intermediate-affinity” IL-2Rβγ complex modulates the activation and proliferation of CD8⁺ effector T (Teff) cells, NK cells, and NKT cells. CD8⁺ Teff cells (also known as cytotoxic T cells, Tc cells, cytotoxic T lymphocytes, CTLs, T-killer cells, cytolytic T cells, Tcon, or killer T cells) are T lymphocytes that recognize and kill damaged cells, cancerous cells, and pathogen-infected cells. NK and NKT cells are types of lymphocytes that, similar to CD8⁺ Teff cells, target cancerous cells and pathogen-infected cells.

In some instances, IL-2 signaling is utilized to modulate T cell responses and subsequently for treatment of a cancer. For example, IL-2 is administered in a high-dose form to induce expansion of Teff cell populations for treatment of a cancer. However, high-dose IL-2 further leads to concomitant stimulation of Treg cells that dampen anti-tumor immune responses. High-dose IL-2 also induces toxic adverse events mediated by the engagement of IL-2R alpha chain-expressing cells in the vasculature, including type 2 innate immune cells (ILC-2), eosinophils and endothelial cells. This leads to eosinophilia, capillary leak and vascular leak syndrome (VLS).

Adoptive cell therapy enables physicians to effectively harness a patient's own immune cells to fight diseases such as proliferative disease (e.g., cancer) as well as infectious disease. The effect of IL-2 signaling may be further enhanced by the presence of additional agents or methods in combination therapy.

One attractive therapy for combination with an IL-2 derivative is PD-1/PD-L1-based therapy. Programmed cell death protein 1, also known as PD-1 or CD279, is a cell surface receptor expressed on T cells and pro-B cells which plays a role in regulating the immune system's response to the cells of the human body. PD-1 down-regulates the immune system and promotes self-tolerance by suppressing T cell inflammatory activity. This prevents autoimmune diseases but can also prevent the immune system from killing cancer cells. PD-1 guards against autoimmunity through two mechanisms. First, PD-1 promotes apoptosis (programmed cell death) of antigen-specific T-cells in lymph nodes. Second, PD-1 reduces apoptosis in regulatory T cells (anti-inflammatory, suppressive T cells). Binding of PD-L1, a protein which is overexpressed on certain cancer cells, to PD-1 prevents T cells from killing the PD-L1-containing cells. Pembrolizumab is a humanized anti-PD-1 antibody that can block PD-1, activate the immune system to attack tumors, and is approved for treatment of certain cancers.

Another attractive therapy is further combining the IL-2 derivative and PD-1/PD-L1-based therapy with cetuximab. Cetuximab is a monoclonal antibody that binds to epidermal growth factor receptor (EGFR). EGFR is a cell surface receptor overexpressed in many types of cancer. Activation of EGFR promotes cell proliferation and survival, as well as angiogenesis, leading to tumor growth and metastasis. Cell growth and angiogenesis may be regulated by blocking the binding of EGFR to epidermal growth factor (EGF). By preventing EGF from binding to EGFR, the downstream signal transduction cascade is inhibited, leading to decreased cell growth. The same effect can be achieved by inhibiting transforming growth factor alpha (TGF-α) from binding to EGFR. The mechanism of action of cetuximab appears to include antibody dependent cell mediated cytotoxicity (Iannello, A. et al., Cancer Metastasis Rev. 2005, 24(4):487-99, the disclosure of which is incorporated herein by reference) in addition to EGFR blockade, which may contribute to its efficacy and may be further exploited. Cetuximab is indicated for the treatment of locally or regionally advanced squamous cell carcinoma of the head and neck in combination with radiation therapy; recurrent locoregional disease or metastatic squamous cell carcinoma of the head and neck in combination with platinum-based therapy with fluorouracil; and recurrent or metastatic squamous cell carcinoma of the head and neck.

A still further therapy is further combining the IL-2 derivative and PD-1/PD-L1-based therapy with an anti-transforming growth factor beta (TGFβ) antibody. TGFβ is a cytokine that has an important physiological role in the regulation of cell proliferation, differentiation, extracellular matrix production, angiogenesis, embryonic development, and immune regulation. Each of these mechanisms is associated with tumor promotion and metastasis. In addition, Treg cells are one of the key immune suppressive cell types within the tumor microenvironment. TGFβ is responsible for the development, maintenance, and function of Treg cells. Inhibition of TGFβ has the potential to treat various malignancies.

Head and neck squamous cell carcinoma (HNSCC) is the ninth leading cancer by incidence worldwide and constitutes 90% of all head and neck cancers (Gupta et al. Oncology, 2016, 91(1):13-23, the disclosure of which is incorporated herein by reference). HNSCC is a biologically diverse and genomically heterogeneous disease that arises from the squamous mucosal lining of the upper aerodigestive tract, including the lip and oral cavity, nasal cavity, paranasal sinuses, nasopharynx, oropharynx, larynx and hypopharynx. A large number of patients with head and neck cancer initially present with locally advanced, Stage III/IV disease that is initially treated with combinations of chemotherapy, radiation and/or surgery. This initial treatment can result in disease control rates ranging between 33% and 86% of patients. Patients who progress after initial therapy require subsequent treatment for recurrent (R) or metastatic (M) disease.

### IL-2 Conjugates

Provided herein are methods of treating HNSCC in a subject in need thereof, comprising administering to the subject an IL-2 conjugate in combination with one or more additional agents.

In some embodiments, the IL-2 sequence comprises the sequence of SEQ ID NO: 1:

(SEQ ID NO: 1)
PTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGS
ETTFMCEYADETATIVEFLNRWITFSQSIISTLT

wherein the amino acid at position P64 is replaced by the structure of Formula (I):

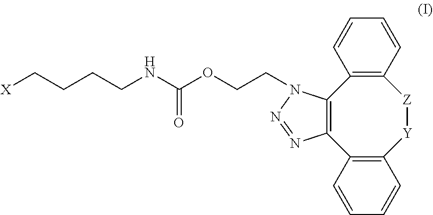

wherein:
Z is CH₂ and Y is

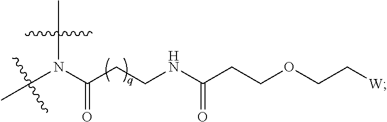

Y is CH₂ and Z is

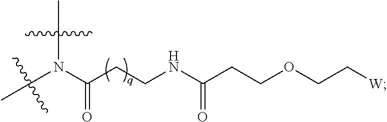

Z is CH₂ and Y is

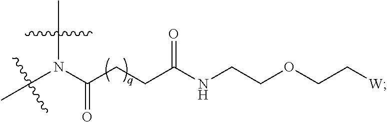

or
Y is CH₂ and Z is

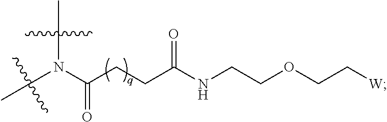

W is a PEG group having an average molecular weight of about 25 kDa-35 kDa;
q is 1, 2, or 3;
X is an L-amino acid having the structure:

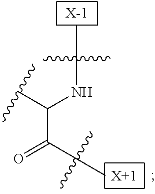

X−1 indicates the point of attachment to the preceding amino acid residue; and
X+1 indicates the point of attachment to the following amino acid residue.

In any of the embodiments or variations of Formula (I) described herein, the IL-2 conjugate is a pharmaceutically acceptable salt, solvate, or hydrate. In some embodiments, the IL-2 conjugate is a pharmaceutically acceptable salt. In some embodiments, the IL-2 conjugate is a solvate. In some embodiments, the IL-2 conjugate is a hydrate.

In any of the embodiments or variations of Formula (I) described herein and pharmaceutical compositions comprising the same, average molecular weight encompasses both weight average molecular weight and number average molecular weight; in other words, for example, both a 30 kDa number average molecular weight and a 30 kDa weight average molecular weight qualify as a 30 kDa molecular weight. In some embodiments, the average molecular weight is weight average molecular weight. In other embodiments, the average molecular weight is number average molecular weight. It is understood that in the methods provided herein, administering an IL-2 conjugate as described herein to a subject comprises administering more than a single molecule of IL-2 conjugate; as such, use of the term “average” to describe the molecular weight of the PEG group refers to the average molecular weight of the PEG groups of the IL-2 conjugate molecules in a dose administered to the subject.

In some embodiments of Formula (I), Z is CH₂ and Y is

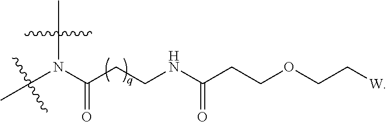

In some embodiments of Formula (I), Y is CH₂ and Z is

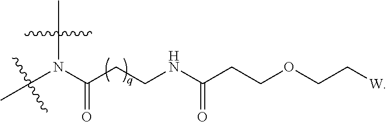

some embodiments of Formula (I), Z is CH₂ and Y is

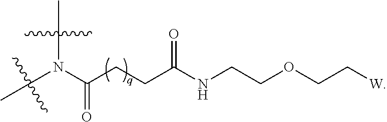

In some embodiments of Formula (I), Y is CH₂ and Z is

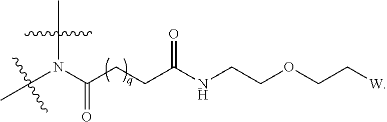

In some embodiments of Formula (I), q is 1. In some embodiments of Formula (I), q is 2. In some embodiments of Formula (I), q is 3.

In some embodiments of Formula (I), W is a PEG group having an average molecular weight of about 25 kDa. In some embodiments of Formula (I), W is a PEG group having an average molecular weight of about 30 kDa. In some embodiments of Formula (I), W is a PEG group having an average molecular weight of about 35 kDa.

In some embodiments of Formula (I), q is 1 and structure of Formula (I) is the structure of Formula (Ia):

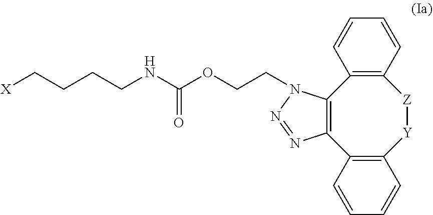

wherein:
Z is CH₂ and Y is

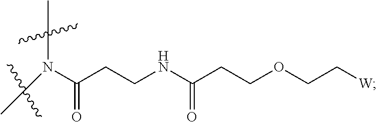

Y is CH₂ and Z is

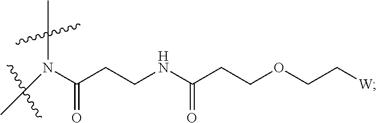

Z is CH₂ and Y is

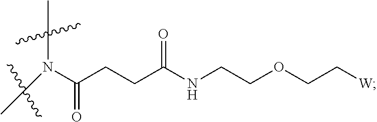

or
Y is CH₂ and Z is

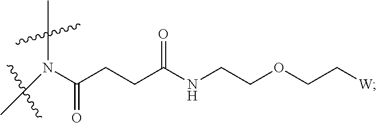

W is a PEG group having an average molecular weight of about 25 kDa-35 kDa;
X is an L-amino acid having the structure:

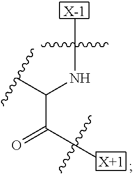

X−1 indicates the point of attachment to the preceding amino acid residue; and
X+1 indicates the point of attachment to the following amino acid residue.

In some embodiments of Formula (Ia), Z is CH₂ and Y is

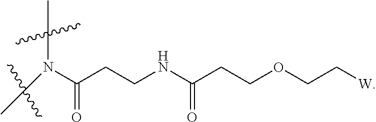

In some embodiments of Formula (Ia), Y is CH₂ and Z is

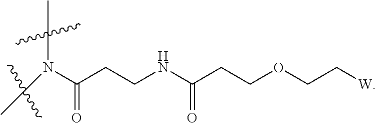

In other embodiments of Formula (Ia), Z is CH₂ and Y is

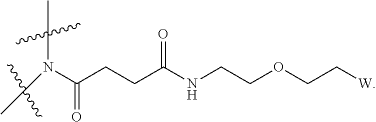

In some embodiments of Formula (Ia), Y is CH₂ and Z is

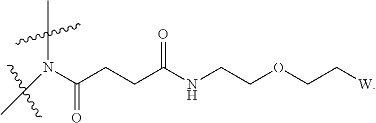

In some embodiments of Formula (Ia), the PEG group has an average molecular weight of about 30 kDa.

In some embodiments, the IL-2 conjugate comprises the sequence of SEQ ID NO: 2:

(SEQ ID NO: 2)
PTSSSTKKTQLQLEHLLLDLQMILNGINNYKNPKLTRMLTFKFYMPKKA
TELKHLQCLEEELK[AzK_L1_PEG30KD]LEEVLNLAQSKNFHLRPRD
LISNINVIVLELKGSETTFMCEYADETATIVEFLNRWITFSQSIISTLT

wherein [AzK_L1_PEG30kD] is N6-((2-azidoethoxy)-carbonyl)-L-lysine stably-conjugated to PEG via DBCO-mediated click chemistry to form a compound comprising a structure of Formula (IV) or Formula (V), wherein q is 1 (such as Formula (IVa) or Formula (Va)), and wherein the PEG group has an average molecular weight of about 25-35 kiloDaltons (e.g., about 30 kDa), capped with a methoxy group. The term “DBCO” means a chemical moiety comprising a dibenzocyclooctyne group, such as comprising the mPEG-DBCO compound illustrated in Schemes 1 and 2 of Example 1.

The ratio of regioisomers generated from the click reaction is about 1:1 or greater than 1:1.

PEGs will typically comprise a number of (OCH₂CH₂) monomers (or (CH₂CH₂O) monomers, depending on how the PEG is defined). In some embodiments, the number of (OCH₂CH₂) monomers (or (CH₂CH₂O) monomers) is such that the average molecular weight of the PEG group is about 30 kDa.

In some instances, the PEG is an end-capped polymer, that is, a polymer having at least one terminus capped with a relatively inert group, such as a lower C₁₋₆ alkoxy group, or a hydroxyl group. In some embodiments, the PEG group is a methoxy-PEG (commonly referred to as mPEG), which is a linear form of PEG wherein one terminus of the polymer is a methoxy (—OCH₃) group, and the other terminus is a hydroxyl or other functional group that can be optionally chemically modified.

In some embodiments, the PEG group is a linear or branched PEG group. In some embodiments, the PEG group is a linear PEG group. In some embodiments, the PEG group is a branched PEG group. In some embodiments, the PEG group is a methoxy PEG group. In some embodiments, the PEG group is a linear or branched methoxy PEG group. In some embodiments, the PEG group is a linear methoxy PEG group. In some embodiments, the PEG group is a branched methoxy PEG group. For example, included within the scope of the present disclosure are IL-2 conjugates comprising a PEG group having a molecular weight of 30,000 Da±3,000 Da, or 30,000 Da±4,500 Da, or 30,000 Da±5,000 Da.

In some embodiments, the IL-2 conjugate comprises the amino acid sequence of SEQ ID NO: 1 in which the amino acid residue P64 is replaced by the structure of Formula (IV) or Formula (V), or a mixture of Formula (IV) and Formula (V):

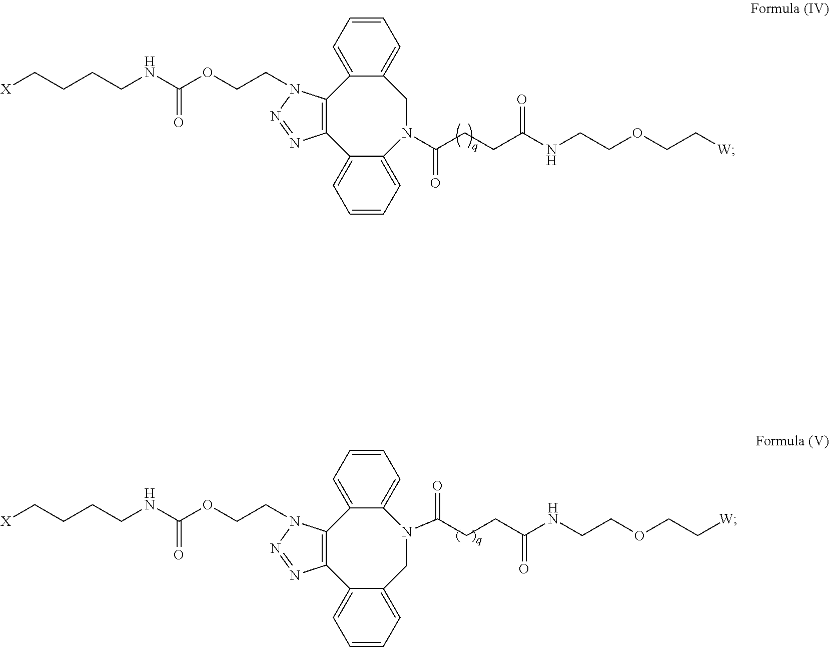

wherein:
W is a PEG group having an average molecular weight of about 25 kDa-35 kDa;
q is 1, 2, or 3; and
X has the structure:

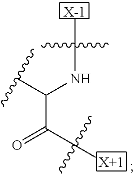

X−1 indicates the point of attachment to the preceding amino acid residue; and
X+1 indicates the point of attachment to the following amino acid residue.

In some embodiments of Formula (IV) or Formula (V), or a mixture of Formula (IV) or Formula (V), q is 1. In some embodiments of Formula (IV) or Formula (V), or a mixture of Formula (IV) or Formula (V), q is 2. In some embodiments of Formula (IV) or Formula (V), or a mixture of Formula (IV) or Formula (V), q is 3.

In some embodiments of Formula (IV) or Formula (V), or a mixture of Formula (IV) or Formula (V), W is a PEG group having an average molecular weight of about 25 kDa. In some embodiments of Formula (IV) or Formula (V), or a mixture of Formula (IV) or Formula (V), W is a PEG group having an average molecular weight of about 30 kDa. In some embodiments of Formula (IV) or Formula (V), or a mixture of Formula (IV) or Formula (V), W is a PEG group having an average molecular weight of about 35 kDa.

In any of the embodiments described herein, the structure of Formula (I) has the structure of Formula (IV) or Formula (V), or is a mixture of Formula (IV) and Formula (V). In some embodiments, the structure of Formula (I) has the structure of Formula (IV). In some embodiments, the structure of Formula (I) has the structure of Formula (V). In some embodiments, the structure of Formula (I) is a mixture of Formula (IV) and Formula (V).

In some embodiments of Formula (IV) or Formula (V), or a mixture of Formula (IV) and Formula (V), q is 1, the structure of Formula (IV) is the structure of Formula (IVa), and the structure of Formula (V) is the structure of Formula (Va):

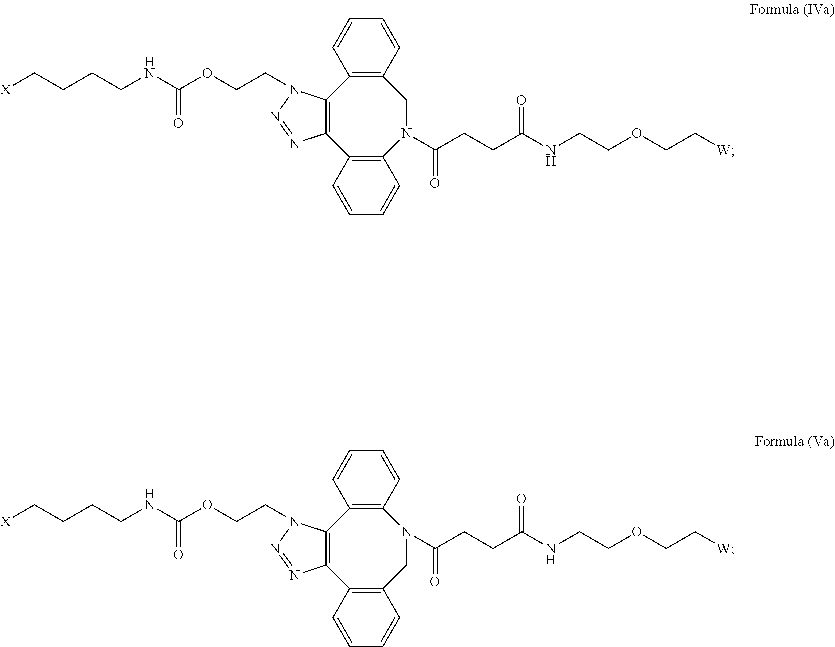

wherein:
W is a PEG group having an average molecular weight of about 25 kDa-35 kDa; and
X has the structure:

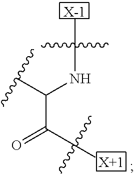

X−1 indicates the point of attachment to the preceding amino acid residue; and
X+1 indicates the point of attachment to the following amino acid residue.

In some embodiments of Formula (IVa) or Formula (Va), or a mixture of Formula (IVa) and Formula (Va), the PEG group has an average molecular weight of about 30 kDa.

In any of the embodiments described herein, the structure of Formula (I) has the structure of Formula (IVa) or Formula (Va), or is a mixture of Formula (IVa) and Formula (Va). In some embodiments, the structure of Formula (I) has the structure of Formula (IVa). In some embodiments, the structure of Formula (I) has the structure of Formula (Va). In some embodiments, the structure of Formula (I) is a mixture of Formula (IVa) and Formula (Va).

In some embodiments, the IL-2 conjugate comprises the amino acid sequence of SEQ ID NO: 1 in which the amino acid residue P64 is replaced by the structure of Formula (XII) or Formula (XIII), or a mixture of Formula (XII) and Formula (XIII):

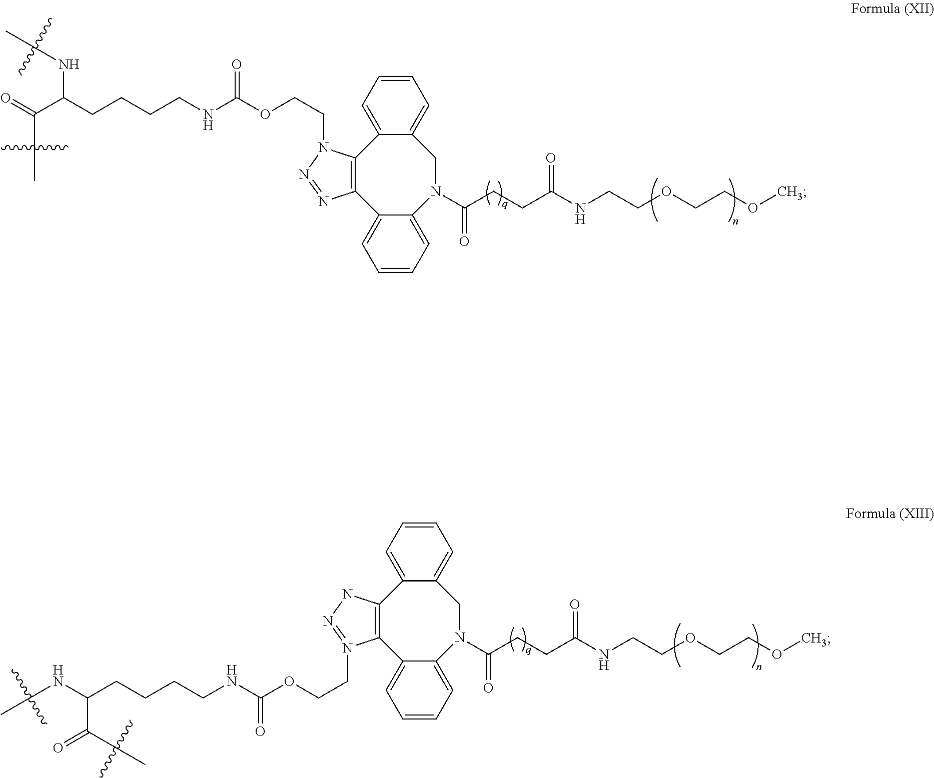

wherein:
n is an integer such that —(OCH₂CH₂)ₙ—OCH₃ has a molecular weight of about 25 kDa-35 kDa;
q is 1, 2, or 3; and
the wavy lines indicate convalent bonds to amino acid residues within SEQ ID NO: 1 that are not replaced.

In some embodiments of Formula (XII) or Formula (XIII), or a mixture of Formula (XII) and Formula (XIII), q is 1. In some embodiments of Formula (XII) or Formula (XIII), or a mixture of Formula (XII) and Formula (XIII), q is 2. In some embodiments of Formula (XII) or Formula (XIII), or a mixture of Formula (XII) and Formula (XIII), q is 3.

In some embodiments of Formula (XII) or Formula (XIII), or a mixture of Formula (XII) and Formula (XIII), n is an integer such that —(OCH₂CH₂)ₙ—OCH₃ has a molecular weight of about 30 kDa.

In any of the embodiments described herein, the structure of Formula (I) has the structure of Formula (XII) or Formula (XIII), or is a mixture of Formula (XII) and Formula (XIII). In some embodiments, the structure of Formula (I) has the structure of Formula (XII). In some embodiments, the structure of Formula (I) has the structure of Formula (XIII). In some embodiments, the structure of Formula (I) is a mixture of Formula (XII) and Formula (XIII).

In some embodiments of Formula (XII) or Formula (XIII), or a mixture of Formula (XII) and Formula (XIII), q is 1, the structure of Formula (XII) is the structure of Formula (XIIa), and the structure of Formula (XIII) is the structure of Formula (XIIIa):

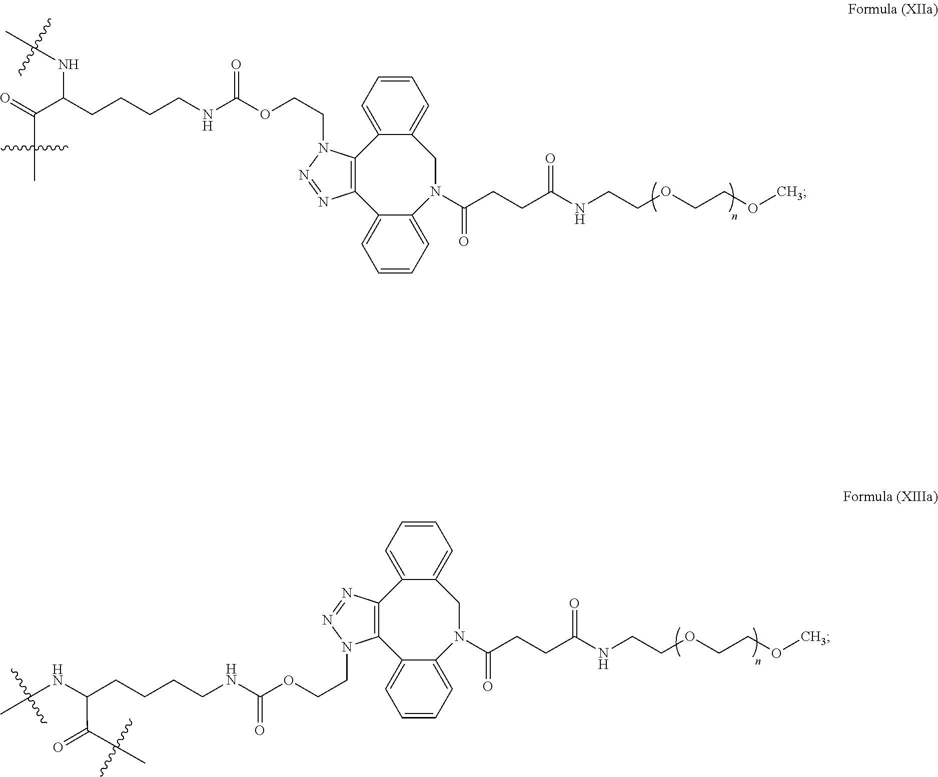

wherein:
n is an integer such that —(OCH₂CH₂)ₙ—OCH₃ has a molecular weight of about 25 kDa-35 kDa; and the wavy lines indicate convalent bonds to amino acid residues within SEQ ID NO: 1 that are not replaced.

In some embodiments of Formula (XIIa) or Formula (XIIIa), or a mixture of Formula (XIIa) and Formula (XIIIa), n is an integer such that —(OCH₂CH₂)ₙ—OCH₃ has a molecular weight of about 30 kDa.

In any of the embodiments described herein, the structure of Formula (I) has the structure of Formula (XIIa) or Formula (XIIIa), or is a mixture of Formula (XIIa) and Formula (XIIIa). In some embodiments, the structure of Formula (I) has the structure of Formula (XIIa). In some embodiments, the structure of Formula (I) has the structure of Formula (XIIIa). In some embodiments, the structure of Formula (I) is a mixture of Formula (XIIa) and Formula (XIIIa).

In some embodiments, the IL-2 conjugate comprises the amino acid sequence of SEQ ID NO: 1 in which the amino acid residue P64 is replaced by the structure of Formula (XIV) or Formula (XV), or a mixture of Formula (XIV) and Formula (XV):

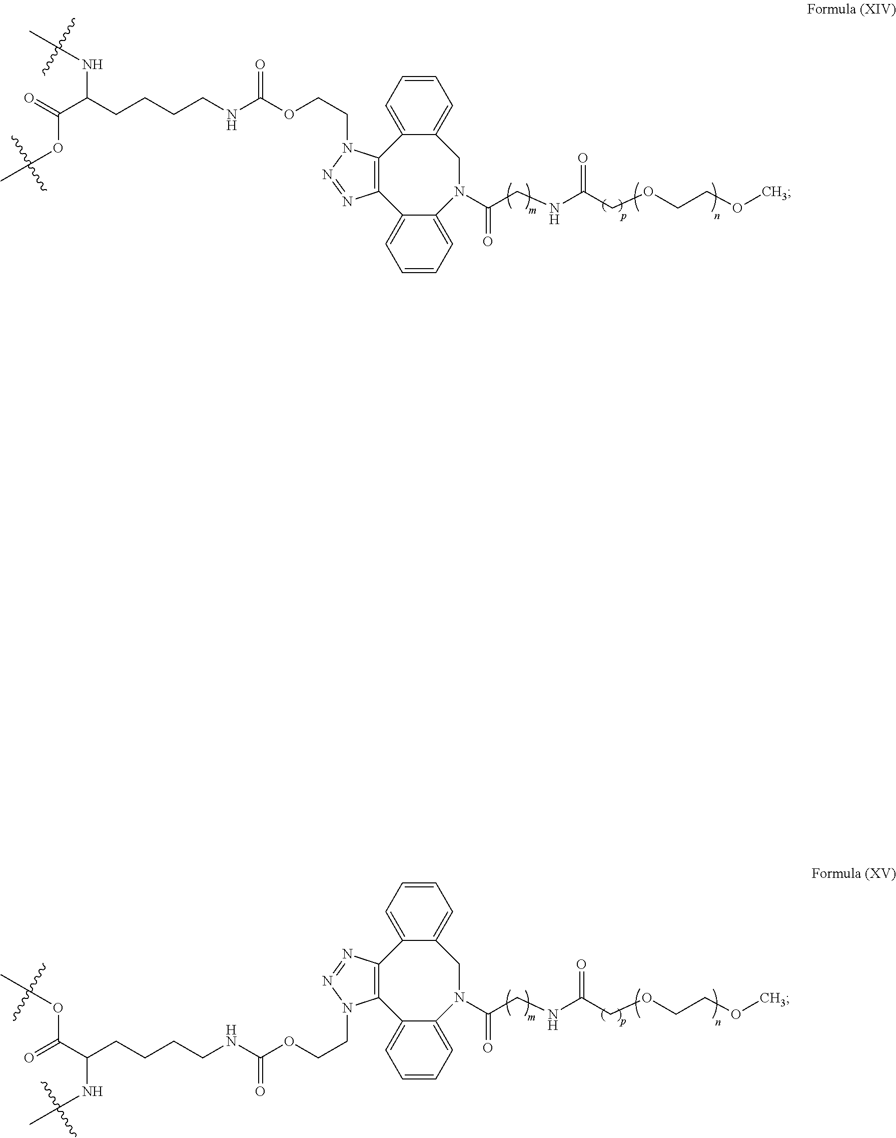

wherein:
m is an integer from 0 to 20;
p is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XIV) or Formula (XV), or a mixture of Formula (XIV) and Formula (XV), n is an integer such that the PEG group has an average molecular weight of about 30 kDa.

In some embodiments, m is an integer from 0 to 15. In some embodiments, m is an integer from 0 to 10. In some embodiments, m is an integer from 0 to 5. In some embodiments, m is an integer from 1 to 5. In some embodiments, m is 1. In some embodiments, m is 2. In some embodiments, m is 3. In some embodiments, m is 4. In some embodiments, m is 5.

In some embodiments, p is an integer from 0 to 15. In some embodiments, p is an integer from 0 to 10. In some embodiments, p is an integer from 0 to 5. In some embodiments, p is an integer from 1 to 5. In some embodiments, p is 1. In some embodiments, p is 2. In some embodiments, p is 3. In some embodiments, p is 4. In some embodiments, p is 5.

In some embodiments, m and p are each 2.

In any of the embodiments described herein, the structure of Formula (I) has the structure of Formula (XIV) or Formula (XV), or is a mixture of Formula (XIV) and Formula (XV). In some embodiments, the structure of Formula (I) has the structure of Formula (XIV). In some embodiments, the structure of Formula (I) has the structure of Formula (XV). In some embodiments, the structure of Formula (I) is a mixture of Formula (XIV) and Formula (XV).

In some embodiments, the IL-2 conjugate comprises the amino acid sequence of SEQ ID NO: 1 in which the amino acid residue P64 is replaced by the structure of Formula (XVI) or Formula (XVII), or a mixture of Formula (XVI) and Formula (XVII):

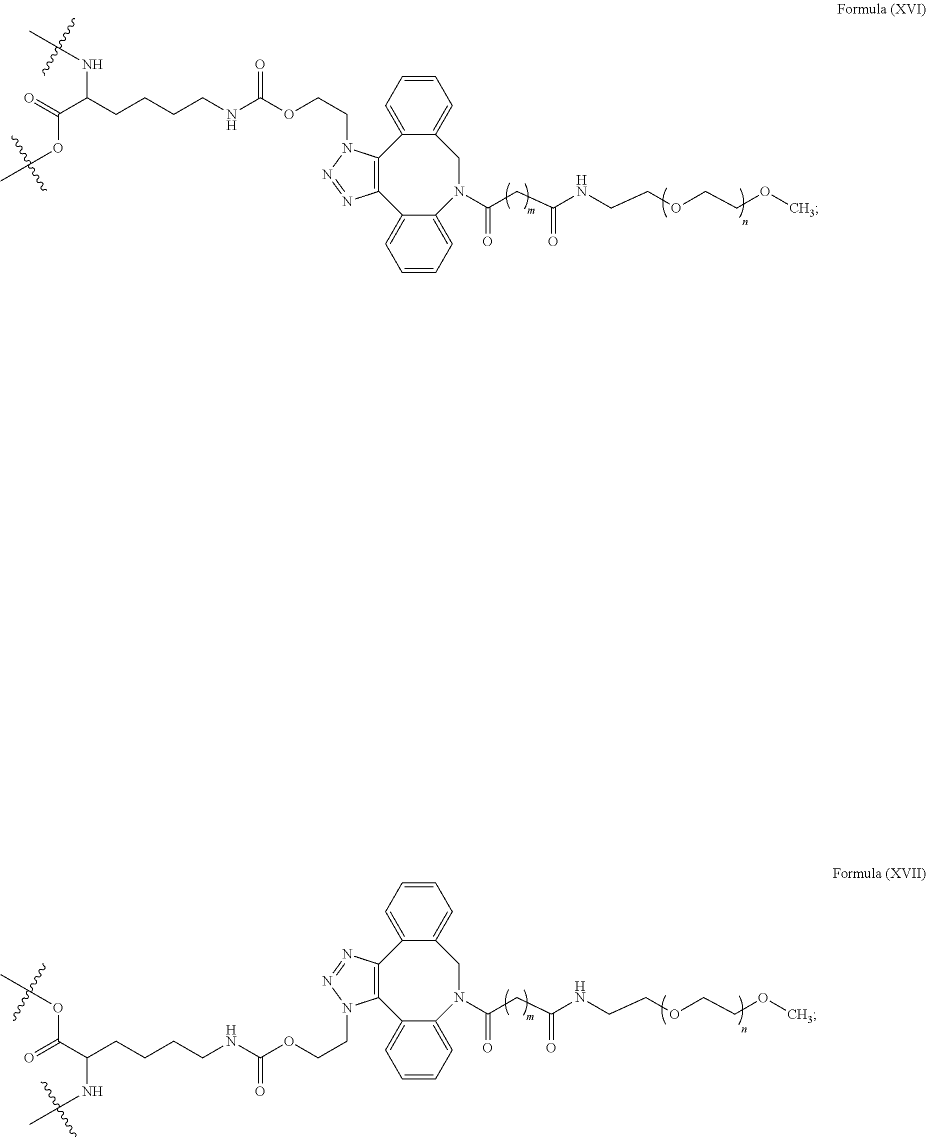

wherein:
m is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XVI) or Formula (XVII), or a mixture of Formula (XVI) and Formula (XVII), n is an integer such that the PEG group has an average molecular weight of about 30 kDa.

In some embodiments, m is an integer from 0 to 15. In some embodiments, m is an integer from 0 to 10. In some embodiments, m is an integer from 0 to 5. In some embodiments, m is an integer from 1 to 5. In some embodiments, m is 1. In some embodiments, m is 2. In some embodiments, m is 3. In some embodiments, m is 4. In some embodiments, m is 5.

In any of the embodiments described herein, the structure of Formula (I) has the structure of Formula (XVI) or Formula (XVII), or is a mixture of Formula (XVI) and Formula (XVII). In some embodiments, the structure of Formula (I) has the structure of Formula (XVI). In some embodiments, the structure of Formula (I) has the structure of Formula (XVII). In some embodiments, the structure of Formula (I) is a mixture of Formula (XVI) and Formula (XVII).

### Conjugation Chemistry

In some embodiments, the IL-2 conjugates described herein can be prepared by a conjugation reaction comprising a 1,3-dipolar cycloaddition reaction. In some embodiments, the 1,3-dipolar cycloaddition reaction comprises reaction of an azide and an alkyne (“Click” reaction). In some embodiments, a conjugation reaction described herein comprises the reaction outlined in Scheme I, wherein X is an unnatural amino acid at position P64 of SEQ ID NO: 1.

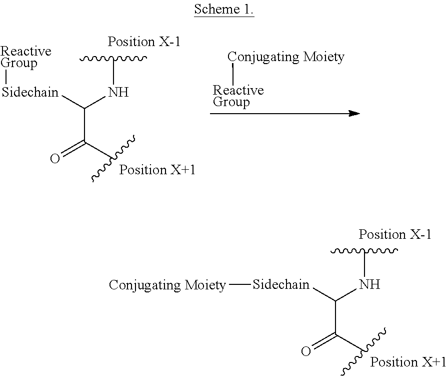

In some embodiments, the conjugating moiety comprises a PEG group as described herein. In some embodiments, a reactive group comprises an alkyne or azide.

In some embodiments, a conjugation reaction described herein comprises the reaction outlined in Scheme II, wherein X is an unnatural amino acid at position P64 of SEQ ID NO: 1.

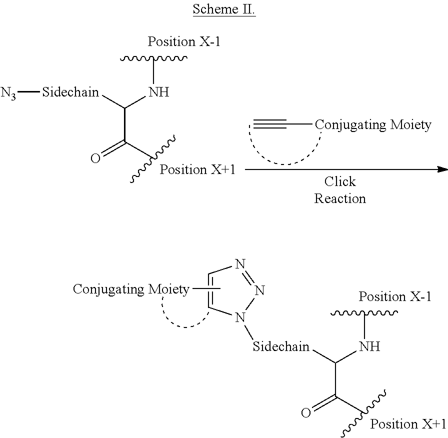

In some embodiments, a conjugation reaction described herein comprises the reaction outlined in Scheme III, wherein X is an unnatural amino acid at position P64 of SEQ ID NO: 1.

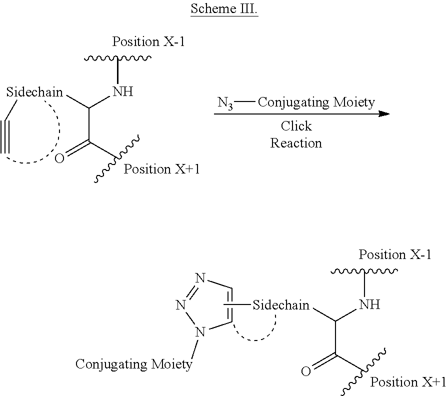

In some embodiments a conjugation reaction described herein comprises the reaction outlined in Scheme IV, wherein X is an unnatural amino acid at position P64 of SEQ ID NO: 1.

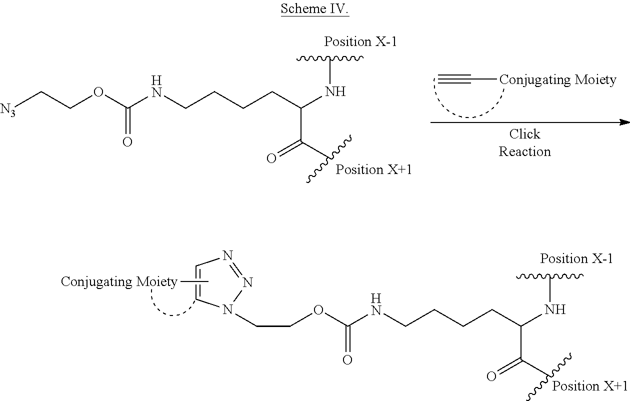

In some embodiments, a conjugation reaction described herein comprises a cycloaddition reaction between an azide moiety, such as that contained in a protein containing an amino acid residue derived from N6-((2-azidoethoxy)-carbonyl)-L-lysine (AzK), and a strained cycloalkyne, such as that derived from DBCO, which is a chemical moiety comprising a dibenzocyclooctyne group. PEG groups comprising a DBCO moiety are commercially available or may be prepared by methods known to those of ordinary skill in the art. Exemplary reactions are shown in Schemes V and VI.

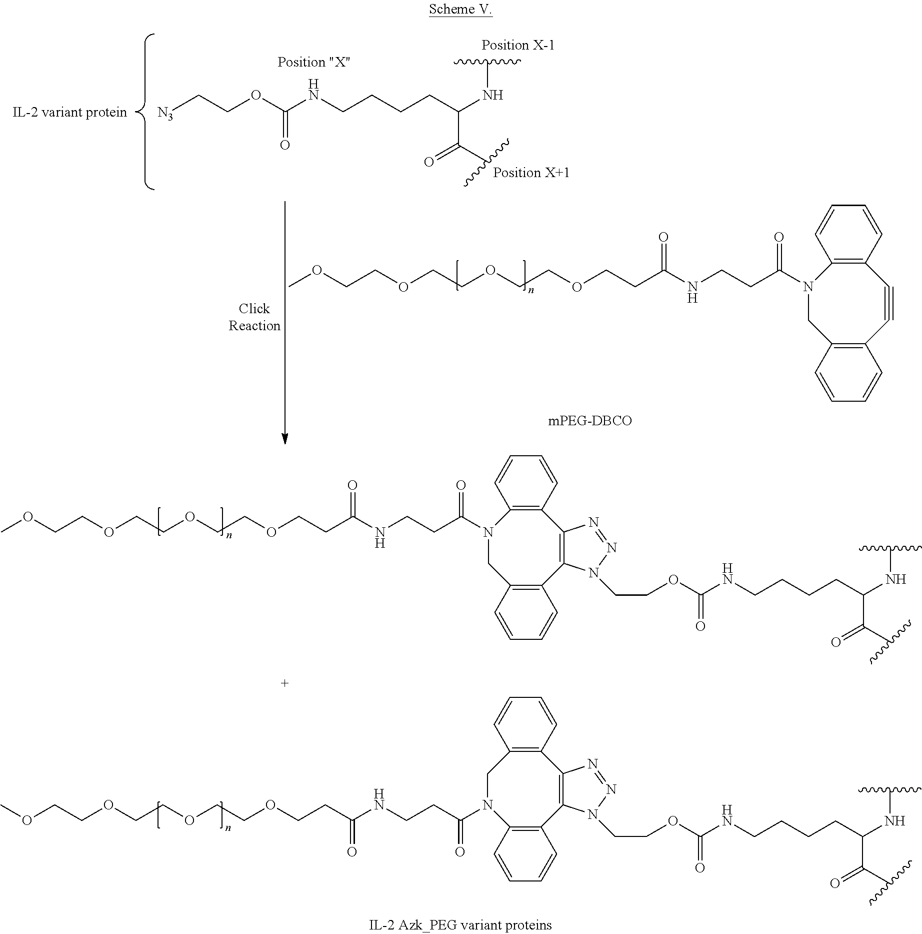

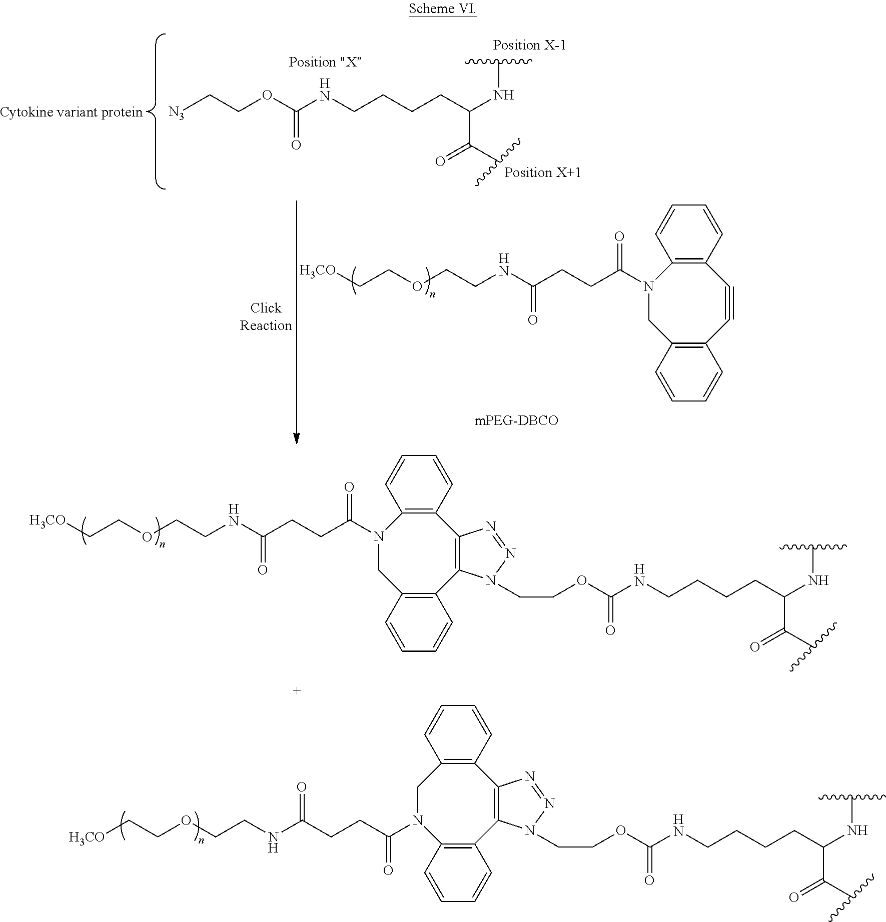

### Cytokine Azk_L1_PEG Variant Proteins

Conjugation reactions such as a click reaction described herein may generate a single regioisomer, or a mixture of regioisomers. In some instances the ratio of regioisomers is about 1:1. In some instances the ratio of regioisomers is about 2:1. In some instances the ratio of regioisomers is about 1.5:1. In some instances the ratio of regioisomers is about 1.2:1. In some instances the ratio of regioisomers is about 1.1:1. In some instances the ratio of regioisomers is greater than 1:1.

### IL-2 Polypeptide Production

In some instances, the IL-2 conjugates described herein, either containing a natural amino acid mutation or an unnatural amino acid mutation, are generated recombinantly or are synthesized chemically. In some instances, IL-2 conjugates described herein are generated recombinantly, for example, either by a host cell system, or in a cell-free system.

In some instances, IL-2 conjugates are generated recombinantly through a host cell system. In some cases, the host cell is a eukaryotic cell (e.g., mammalian cell, insect cells, yeast cells or plant cell) or a prokaryotic cell (e.g., Gram-positive bacterium or a Gram-negative bacterium). In some cases, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™—BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cells include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris (K. phaffii) yeast strains such as GS 115, KM71H, SMD1168, SMD1168H, and X-33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™ MegaX™, DH12S™, INV110, TOP10F′, INVαF, TOP10/P3, ccdB Survival, PIR1, PIR2, Stbl2™, Stbl3™, or Stbl4™

In some instances, suitable polynucleic acid molecules or vectors for the production of an IL-2 polypeptide described herein include any suitable vectors derived from either a eukaryotic or prokaryotic source. Exemplary polynucleic acid molecules or vectors include vectors from bacteria (e.g., E. coli), insects, yeast (e.g., Pichia pastoris, K. phaffii), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris (K. phaffii) vector, pGAPZA, B, & C Pichia pastoris (K. phaffii) vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is used for the production of an IL-2 polypeptide described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, Archaea, or HeLa cells. Exemplary cell-free systems include E. coli S30 Extract system, E. coli T7 S30 system, or PURExpress®, XpressCF, and XpressCF+.

Cell-free translation systems variously comprise components such as plasmids, mRNA, DNA, tRNAs, synthetases, release factors, ribosomes, chaperone proteins, translation initiation and elongation factors, natural and/or unnatural amino acids, and/or other components used for protein expression. Such components are optionally modified to improve yields, increase synthesis rate, increase protein product fidelity, or incorporate unnatural amino acids. In some embodiments, cytokines described herein are synthesized using cell-free translation systems described in U.S. Pat. No. 8,778,631; US 2017/0283469; US 2018/0051065; US 2014/0315245; or U.S. Pat. No. 8,778,631, the disclosure of each of which is incorporated herein by reference. In some embodiments, cell-free translation systems comprise modified release factors, or even removal of one or more release factors from the system. In some embodiments, cell-free translation systems comprise a reduced protease concentration. In some embodiments, cell-free translation systems comprise modified tRNAs with re-assigned codons used to code for unnatural amino acids. In some embodiments, the synthetases described herein for the incorporation of unnatural amino acids are used in cell-free translation systems. In some embodiments, tRNAs are pre-loaded with unnatural amino acids using enzymatic or chemical methods before being added to a cell-free translation system. In some embodiments, components for a cell-free translation system are obtained from modified organisms, such as modified bacteria, yeast, or other organism.

In some embodiments, an IL-2 polypeptide is generated as a circularly permuted form, either via an expression host system or through a cell-free system.

### Production of Cytokine Polypeptide Comprising an Unnatural Amino Acid

An orthogonal or expanded genetic code can be used in the present disclosure, in which one or more specific codons present in the nucleic acid sequence of an IL-2 polypeptide are allocated to encode the unnatural amino acid so that it can be genetically incorporated into the IL-2 by using an orthogonal tRNA synthetase/tRNA pair. The orthogonal tRNA synthetase/tRNA pair is capable of charging a tRNA with an unnatural amino acid and is capable of incorporating that unnatural amino acid into the polypeptide chain in response to the codon.

In some instances, the codon is the codon amber, ochre, opal or a quadruplet codon. In some cases, the codon corresponds to the orthogonal tRNA which will be used to carry the unnatural amino acid. In some cases, the codon is amber. In other cases, the codon is an orthogonal codon.

In some instances, the codon is a quadruplet codon, which can be decoded by an orthogonal ribosome ribo-Q1. In some cases, the quadruplet codon is as illustrated in Neumann, et al., “Encoding multiple unnatural amino acids via evolution of a quadruplet-decoding ribosome,” Nature, 464(7287): 441-444 (2010), the disclosure of which is incorporated herein by reference.

In some instances, a codon used in the present disclosure is a recoded codon, e.g., a synonymous codon or a rare codon that is replaced with alternative codon. In some cases, the recoded codon is as described in Napolitano, et al., “Emergent rules for codon choice elucidated by editing rare arginine codons in Escherichia coli,” PNAS, 113(38): E5588-5597 (2016), the disclosure of which is incorporated herein by reference. In some cases, the recoded codon is as described in Ostrov et al., “Design, synthesis, and testing toward a 57-codon genome,” Science 353(6301): 819-822 (2016), the disclosure of which is incorporated herein by reference.

In some instances, unnatural nucleic acids are utilized leading to incorporation of one or more unnatural amino acids into the IL-2. Exemplary unnatural nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C≡CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993; the disclosure of each of which is incorporated herein by reference.

Unnatural nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d]pyrimidin-5-yl, 2-amino-4-oxopyrolo [2, 3-d] pyrimidin-5-yl, 2-amino-4-oxopyrrolo [2.3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050; the disclosure of each of which is incorporated herein by reference.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337); the disclosure of each of which is incorporated herein by reference.

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)-CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414). The disclosure of each reference listed in this paragraph is incorporated herein by reference.

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R=H, C₁-C₁₂ alkyl or a protecting group); and combinations thereof. Examples of chemically modified sugars can be found in WO2008/101157, US2005/0130923, and WO2007/134181, the disclosure of each of which is incorporated herein by reference.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N— alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, the disclosure of each of which is incorporated herein by reference.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O-(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂-O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393. The disclosure of each reference listed in this paragraph is incorporated herein by reference.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, NY; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179; the disclosure of each of which is incorporated herein by reference.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179, the disclosure of which is incorporated herein by reference). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941. The disclosure of each reference listed in this paragraph is incorporated herein by reference.

In some cases, the unnatural nucleic acids further form unnatural base pairs. Exemplary unnatural nucleotides capable of forming an unnatural DNA or RNA base pair (UBP) under conditions in vivo includes, but is not limited to, TAT1, dTAT1, 5FM, d5FM, TPT3, dTPT3, 5SICS, d5SICS, NaM, dNaM, CNMO, dCNMO, and combinations thereof. In some embodiments, unnatural nucleotides include:

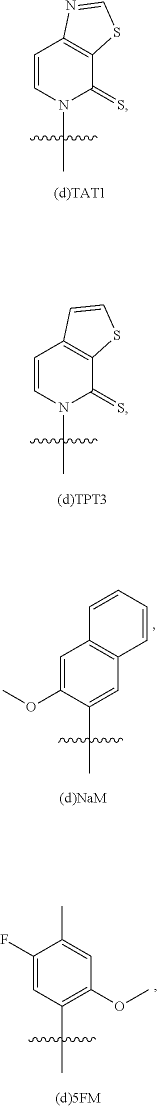

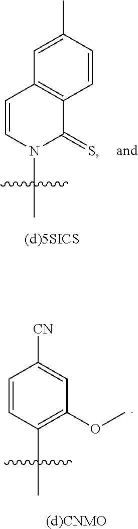 [SMILES: *n1ccc2cc(C)ccc2c1=S ; "(d)5SICS" ; *c1ccc(C#N)cc1OC ; "(d)CNMO"]

Exemplary unnatural base pairs include: (d)TPT3-(d)NaM; (d)5SICS-(d)NaM; (d)CNMO-(d)TAT 1; (d)NaM-(d)TAT1; (d)CNMO-(d)TPT3; and (d)5FM-(d)TAT1.

Other examples of unnatural nucleotides capable of forming unnatural UBPs that may be used to prepare the IL-2 conjugates disclosed herein may be found in Dien et al., J Am Chem Soc., 2018, 140:16115-16123; Feldman et al., J Am Chem Soc, 2017, 139:11427-11433; Ledbetter et al., J Am Chem Soc., 2018, 140:758-765; Dhami et al., Nucleic Acids Res. 2014, 42:10235-10244; Malyshev et al., Nature, 2014, 509:385-388; Betz et al., J Am Chem Soc., 2013, 135:18637-18643; Lavergne et al., J Am Chem Soc. 2013, 135:5408-5419; and Malyshev et al. Proc Natl Acad Sci USA, 2012, 109:12005-12010; the disclosure of each of which is incorporated herein by reference. In some embodiments, unnatural nucleotides include:

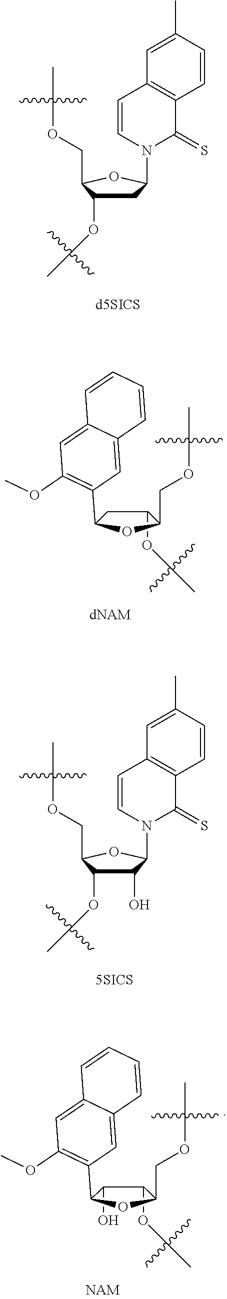

In some embodiments, the unnatural nucleotides that may be used to prepare the IL-2 conjugates disclosed herein may be derived from a compound of the formula

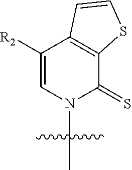

wherein R₂ is selected from the group consisting of hydrogen, alkyl, alkenyl, alkynyl, methoxy, methanethiol, methaneseleno, halogen, cyano, and azido; and
the wavy line indicates a bond to a ribosyl or 2′-deoxyribosyl, wherein the 5′-hydroxy group of the ribosyl or 2′-deoxyribosyl moiety is in free form, is connected to a monophosphate, diphosphate, triphosphate, α-thiotriphosphate, β-thiotriphosphate, or γ-thiotriphosphate group, or is included in an RNA or a DNA or in an RNA analog or a DNA analog.

In some embodiments, the unnatural nucleotides that may be used to prepare the IL-2 conjugates disclosed herein may be derived from a compound of the Formula

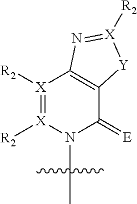

wherein:
each X is independently carbon or nitrogen;
R₂ is absent when X is nitrogen, and is present when X is carbon and is independently hydrogen, alkyl, alkenyl, alkynyl, methoxy, methanethiol, methaneseleno, halogen, cyano, or azide;
Y is sulfur, oxygen, selenium, or secondary amine;
E is oxygen, sulfur, or selenium; and
the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, is connected to a mono-phosphate, diphosphate, triphosphate, α-thiotriphosphate, β-thiotriphosphate, or γ-thiotriphosphate group, or is included in an RNA or a DNA or in an RNA analog or a DNA analog.

In some embodiments, each X is carbon. In some embodiments, at least one X is carbon. In some embodiments, one X is carbon. In some embodiments, at least two X are carbon. In some embodiments, two X are carbon. In some embodiments, at least one X is nitrogen. In some embodiments, one X is nitrogen. In some embodiments, at least two X are nitrogen. In some embodiments, two X are nitrogen.

In some embodiments, Y is sulfur. In some embodiments, Y is oxygen. In some embodiments, Y is selenium. In some embodiments, Y is a secondary amine.

In some embodiments, E is sulfur. In some embodiments, E is oxygen. In some embodiments, E is selenium.

In some embodiments, R₂ is present when X is carbon. In some embodiments, R² is absent when X is nitrogen. In some embodiments, each R₂, where present, is hydrogen. In some embodiments, R₂ is alkyl, such as methyl, ethyl, or propyl. In some embodiments, R₂ is alkenyl, such as —CH₂═CH₂. In some embodiments, R₂ is alkynyl, such as ethynyl. In some embodiments, R₂ is methoxy. In some embodiments, R₂ is methanethiol. In some embodiments, R₂ is methaneseleno. In some embodiments, R₂ is halogen, such as chloro, bromo, or fluoro. In some embodiments, R₂ is cyano. In some embodiments, R₂ is azide.

In some embodiments, E is sulfur, Y is sulfur, and each X is independently carbon or nitrogen. In some embodiments, E is sulfur, Y is sulfur, and each X is carbon.

In some embodiments, the unnatural nucleotides that may be used to prepare the IL-2 conjugates disclosed herein may be derived from

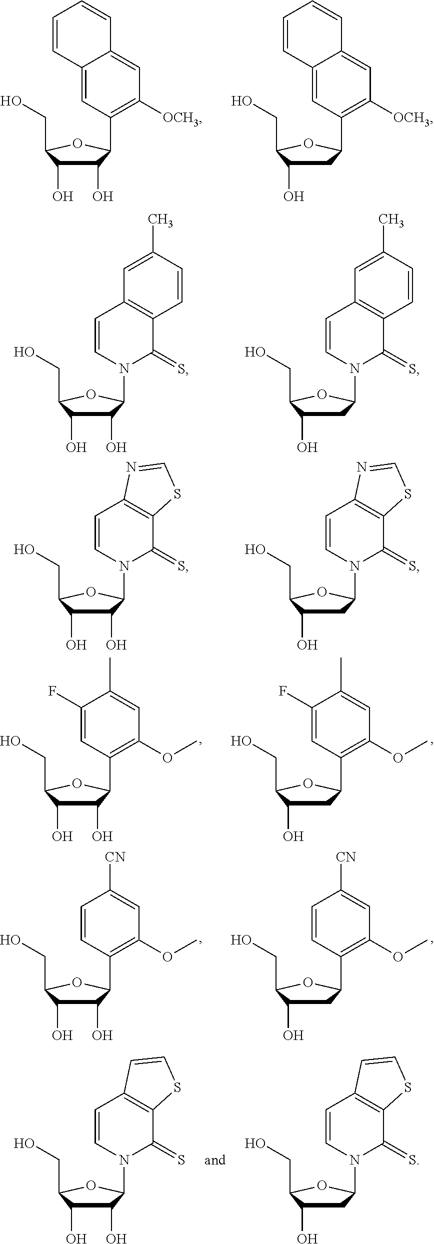

In some embodiments, the unnatural nucleotides that may be used to prepare the IL-2 conjugates disclosed herein include

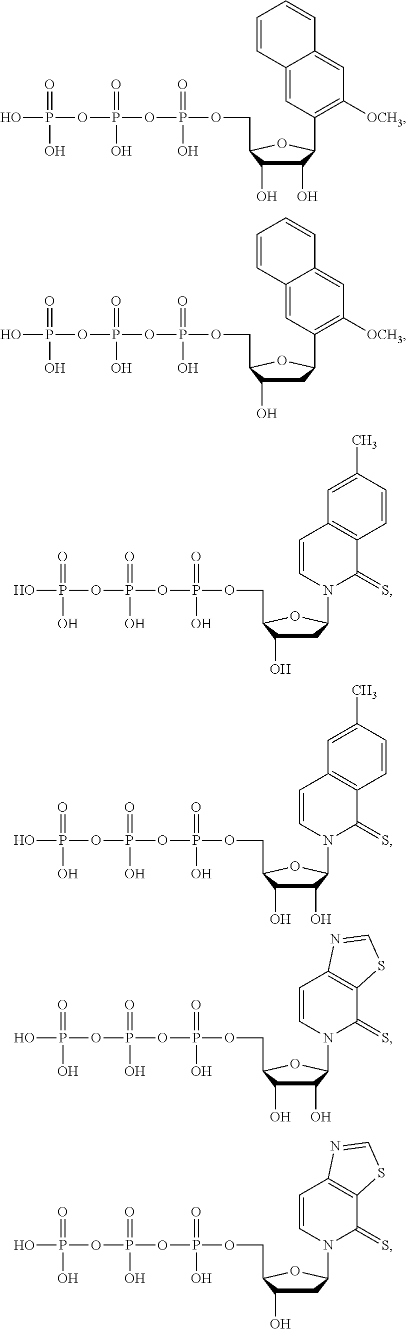

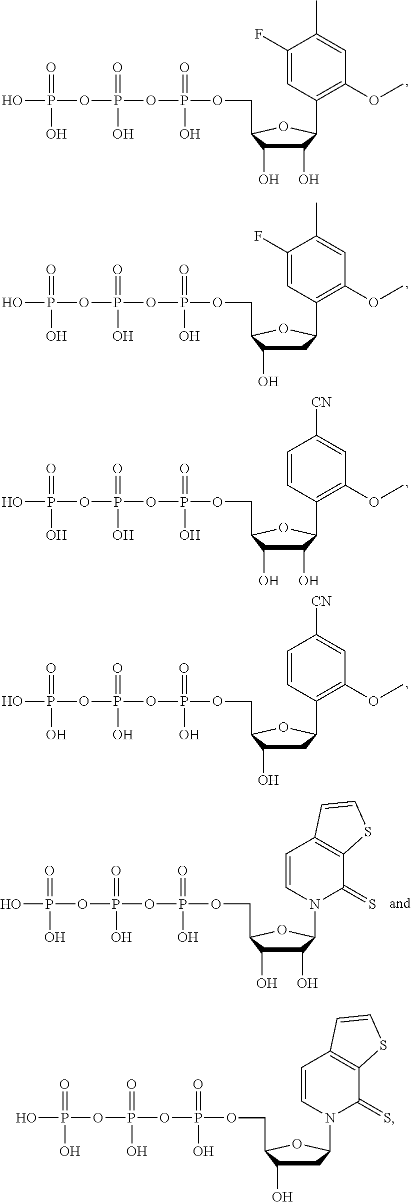

In some embodiments, an unnatural base pair generate an unnatural amino acid described in Dumas et al., “Designing logical codon reassignment—Expanding the chemistry in biology,” Chemical Science, 6: 50-69 (2015), the disclosure of which is incorporated herein by reference.

In some embodiments, the unnatural amino acid is incorporated into the cytokine (e.g., the IL polypeptide) by a synthetic codon comprising an unnatural nucleic acid. In some instances, the unnatural amino acid is incorporated into the cytokine by an orthogonal, modified synthetase/tRNA pair. Such orthogonal pairs comprise an unnatural synthetase that is capable of charging the unnatural tRNA with the unnatural amino acid, while minimizing charging of a) other endogenous amino acids onto the unnatural tRNA and b) unnatural amino acids onto other endogenous tRNAs. Such orthogonal pairs comprise tRNAs that are capable of being charged by the unnatural synthetase, while avoiding being charged with a) other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of two different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid is incorporated into the cytokine (e.g., the IL polypeptide) by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. In some instances, the unnatural amino acid is incorporated into the cytokine (e.g., the IL polypeptide) by a Mj-TyrRS/tRNA pair. Exemplary UAAs that can be incorporated by a Mj-TyrRS/tRNA pair include, but are not limited to, para-substituted phenylalanine derivatives such asp-aminophenylalanine and p-methoyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronophenylalanine; and o-nitrobenzyltyrosine.

In some instances, the unnatural amino acid is incorporated into the cytokine (e.g., the IL polypeptide) by a Ec-Tyr/tRNA_{CUA} or a Ec-Leu/tRNA_{CUA} pair. Exemplary UAAs that can be incorporated by a Ec-Tyr/tRNA_{CUA} or a Ec-Leu/tRNA_{CUA} pair include, but are not limited to, phenylalanine derivatives containing benzophenone, ketone, iodide, or azide substituents; O-propargyltyrosine; α-aminocaprylic acid, O-methyl tyrosine, 0-nitrobenzyl cysteine; and 3-(naphthalene-2-ylamino)-2-amino-propanoic acid.

In some instances, the unnatural amino acid is incorporated into the cytokine (e.g., the IL polypeptide) by a pyrrolysyl-tRNA pair. In some cases, the PylRS is obtained from an archaebacterial, e.g., from a methanogenic archaebacterial. In some cases, the PylRS is obtained from Methanosarcina barkeri, Methanosarcina mazei, or Methanosarcina acetivorans. Exemplary UAAs that can be incorporated by a pyrrolysyl-tRNA pair include, but are not limited to, amide and carbamate substituted lysines such as 2-amino-6-((R)-tetrahydrofuran-2-carboxamido)hexanoic acid, N-ε-_{D}-prolyl-_{L}-lysine, and N-ε-cyclopentyloxycarbonyl-_{L}-lysine; N—ε-Acryloyl-_{L}-lysine; N-ε-[(1-(6-nitrobenzo[d][1,3]dioxol-5-yl)ethoxy)carbonyl]-_{L}-lysine; and N-ε-(1-methylcyclopro-2-enecarboxamido)lysine. In some embodiments, the IL-2 conjugates disclosed herein may be prepared by use of M. mazei tRNA which is selectively charged with a non-natural amino acid such as N6-((2-azidoethoxy)-carbonyl)-L-lysine (AzK) by the M. barkeri pyrrolysyl-tRNA synthetase (Mb PylRS). Other methods are known to those of ordinary skill in the art, such as those disclosed in Zhang et al., Nature 2017, 551(7682): 644-647, the disclosure of which is incorporated herein by reference.

In some instances, an unnatural amino acid is incorporated into a cytokine described herein (e.g., the IL polypeptide) by a synthetase disclosed in U.S. Pat. Nos. 9,988,619 and 9,938,516, the disclosure of each of which is incorporated herein by reference.

The host cell into which the constructs or vectors disclosed herein are introduced is cultured or maintained in a suitable medium such that the tRNA, the tRNA synthetase and the protein of interest are produced. The medium also comprises the unnatural amino acid(s) such that the protein of interest incorporates the unnatural amino acid(s). In some embodiments, a nucleoside triphosphate transporter (NTT) from bacteria, plant, or algae is also present in the host cell. In some embodiments, the IL-2 conjugates disclosed herein are prepared by use of a host cell that expresses a NTT. In some embodiments, the nucleotide nucleoside triphosphate transporter used in the host cell may be selected from TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT1, PamNTT2 (Protochlamydia amoebophila), CcNTT (Caedibacter caryophilus), RpNTT1 (Rickettsia prowazekii). In some embodiments, the NTT is selected from PtNTT1, PtNTT2, PtNTT3, PtNTT4, PtNTT5, and PtNTT6. In some embodiments, the NTT is PtNTT1. In some embodiments, the NTT is PtNTT2. In some embodiments, the NTT is PtNTT3. In some embodiments, the NTT is PtNTT4. In some embodiments, the NTT is PtNTT5. In some embodiments, the NTT is PtNTT6. Other NTTs that may be used are disclosed in Zhang et al., Nature 2017, 551(7682): 644-647; Malyshev et al. Nature 2014 (509(7500), 385-388; and Zhang et al. Proc Natl Acad Sci USA, 2017, 114:1317-1322, the disclosure of each of which is incorporated herein by reference.

The orthogonal tRNA synthetase/tRNA pair charges a tRNA with an unnatural amino acid and incorporates the unnatural amino acid into the polypeptide chain in response to the codon. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. Other aaRS-tRNA pairs that may be used according to the present disclosure include those derived from M. mazei those described in Feldman et al., J Am Chem Soc., 2018 140:1447-1454; and Zhang et al. Proc Natl Acad Sci USA, 2017, 114:1317-1322; the disclosure of each of which is incorporated herein by reference.

In some embodiments are provided methods of preparing the IL-2 conjugates disclosed herein in a cellular system that expresses a NTT and a tRNA synthetase. In some embodiments described herein, the NTT is selected from PtNTT1, PtNTT2, PtNTT3, PtNTT4, PtNTT5, and PtNTT6, and the tRNA synthetase is selected from Methanococcus jannaschii, E. coli TyrRS (Ec-Tyr)/B. stearothermophilus, and M. mazei. In some embodiments, the NTT is PtNTT1 and the tRNA synthetase is derived from Methanococcus jannaschii, E. coli TyrRS (Ec-Tyr)/B. stearothermophilus, or M. mazei. In some embodiments, the NTT is PtNTT2 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT4 and the tRNA synthetase is derived from Methanococcus jannaschii, E. coli TyrRS (Ec-Tyr)/B. stearothermophilus, or M. mazei. In some embodiments, the NTT is PtNTT5 and the tRNA synthetase is derived from Methanococcus jannaschii, E. coli TyrRS (Ec-Tyr)/B. stearothermophilus, or M. mazei. In some embodiments, the NTT is PtNTT6 and the tRNA synthetase is derived from Methanococcus jannaschii, E. coli TyrRS (Ec-Tyr)/B. stearothermophilus, or M. mazei.

In some embodiments, the IL-2 conjugates disclosed herein may be prepared in a cell, such as E. coli, comprising (a) nucleotide triphosphate transporter PtNTT2 (including a truncated variant in which the first 65 amino acid residues of the full-length protein are deleted), (b) a plasmid comprising a double-stranded oligonucleotide that encodes an IL-2 variant having a desired amino acid sequence and that contains a unnatural base pair comprising a first unnatural nucleotide and a second unnatural nucleotide to provide a codon at the desired position at which an unnatural amino acid, such as N6-((2-azidoethoxy)-carbonyl)-L-lysine (AzK), will be incorporated, (c) a plasmid encoding a tRNA derived from M. mazei and which comprises an unnatural nucleotide to provide a recognized anticodon (to the codon of the IL-2 variant) in place of its native sequence, and (d) a plasmid encoding a M. barkeri derived pyrrolysyl-tRNA synthetase (Mb PylRS), which may be the same plasmid that encodes the tRNA or a different plasmid. In some embodiments, the cell is further supplemented with deoxyribo triphosphates comprising one or more unnatural bases. In some embodiments, the cell is further supplemented with ribo triphosphates comprising one or more unnatural bases. In some embodiments, the cells is further supplemented with one or more unnatural amino acids, such as N6-((2-azidoethoxy)-carbonyl)-L-lysine (AzK). In some embodiments, the double-stranded oligonucleotide that encodes the amino acid sequence of the desired IL-2 variant contains a codon AXC at position 64 of the sequence that encodes the protein having SEQ ID NO: 1, wherein X is an unnatural nucleotide. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide that is complementary and may be the same or different as the unnatural nucleotide in the codon. In some embodiments, the unnatural nucleotide in the codon is different than and complimentary to the unnatural nucleotide in the anti-codon. In some embodiments, the unnatural nucleotide in the codon is the same as the unnatural nucleotide in the anti-codon. In some embodiments, the first and second unnatural nucleotides comprising the unnatural base pair in the double-stranded oligonucleotide may be derived from

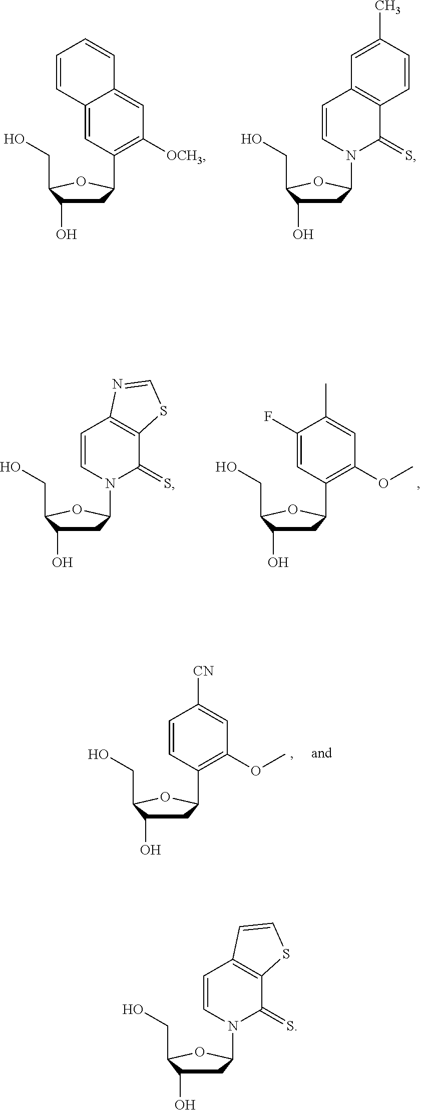

In some embodiments, the first and second unnatural nucleotides comprising the unnatural base pair in the double-stranded oligonucleotide may be derived from

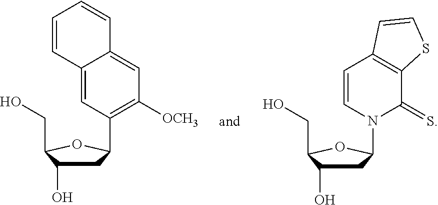

In some embodiments, the triphosphates of the first and second unnatural nucleotides include,

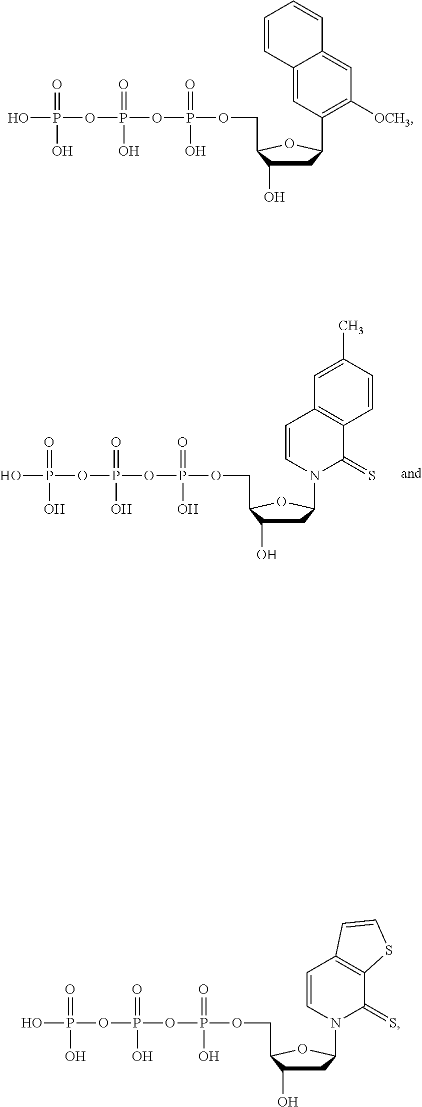

or salts thereof. In some embodiments, the triphosphates of the first and second unnatural nucleotides include,

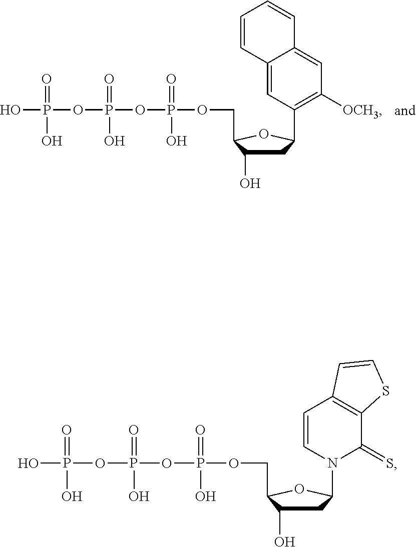

or salts thereof. In some embodiments, the mRNA derived the double-stranded oligonucleotide comprising a first unnatural nucleotide and a second unnatural nucleotide may comprise a codon comprising an unnatural nucleotide derived from

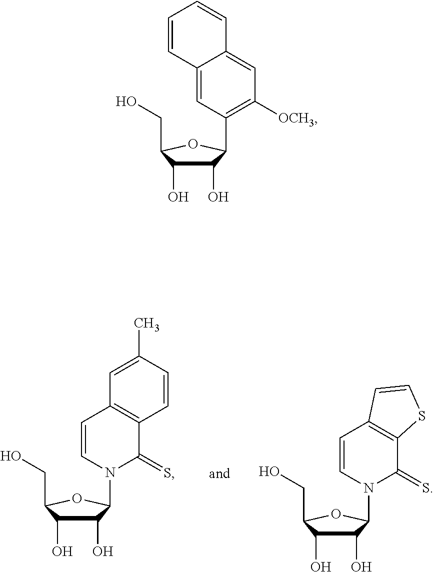

In some embodiments, the M. mazei tRNA may comprise an anti-codon comprising an unnatural nucleotide that recognizes the codon comprising the unnatural nucleotide of the mRNA. The anti-codon in the M. mazei tRNA may comprise an unnatural nucleotide derived from

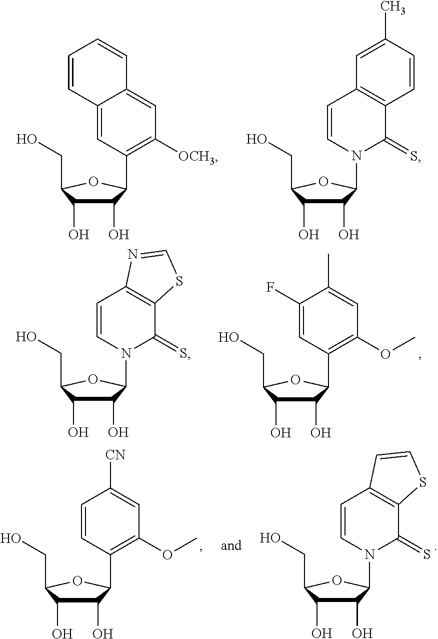

In some embodiments, the mRNA comprises an unnatural nucleotide derived from

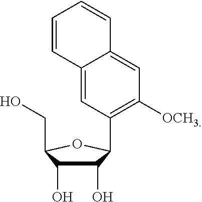

In some embodiments, the mRNA comprises an unnatural nucleotide derived from

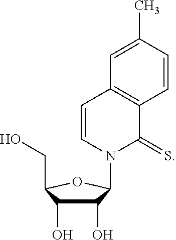

In some embodiments, the mRNA comprises an unnatural nucleotide derived from

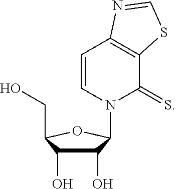

In some embodiments, the mRNA comprises an unnatural nucleotide derived from

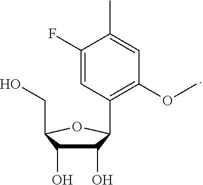

In some embodiments, the mRNA comprises an unnatural nucleotide derived from

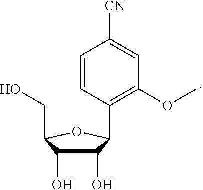

In some embodiments, the mRNA comprises an unnatural nucleotide derived from

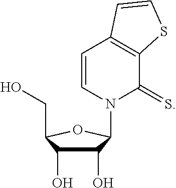

In some embodiments, the tRNA comprises an unnatural nucleotide derived from

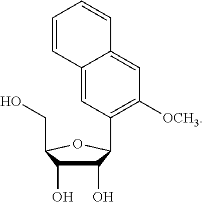

In some embodiments, the tRNA comprises an unnatural nucleotide derived from

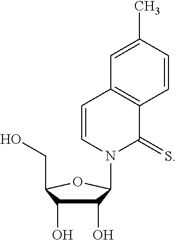

In some embodiments, the tRNA comprises an unnatural nucleotide derived from

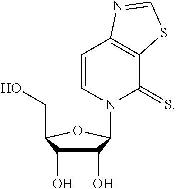

In some embodiments, the tRNA comprises an unnatural nucleotide derived from

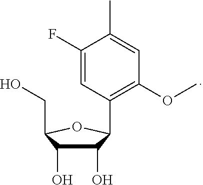

In some embodiments, the tRNA comprises an unnatural nucleotide derived from

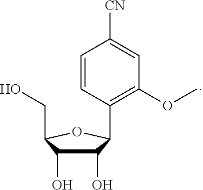

In some embodiments, the tRNA comprises an unnatural nucleotide derived from

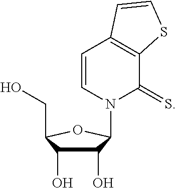

In some embodiments, the mRNA comprises an unnatural nucleotide derived from

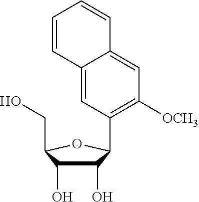

and the tRNA comprises an unnatural nucleotide derived from

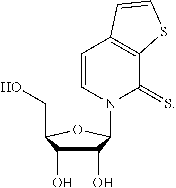

In some embodiments, the mRNA comprises an unnatural nucleotide derived from

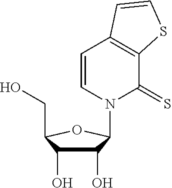

and the tRNA comprises an unnatural nucleotide derived from

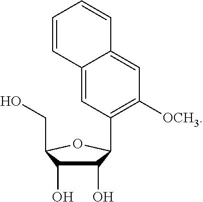

In some embodiments, the mRNA comprises an unnatural nucleotide derived from

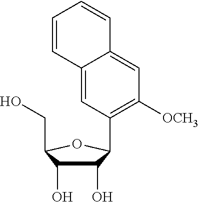

and the tRNA comprises an unnatural nucleotide derived from

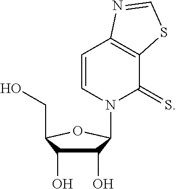

In some embodiments, the mRNA comprises an unnatural nucleotide derived from

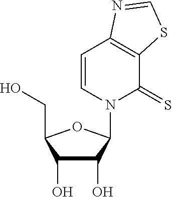

and the tRNA comprises an unnatural nucleotide derived from

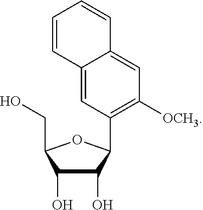

The host cell is cultured in a medium containing appropriate nutrients, and is supplemented with (a) the triphosphates of the deoxyribo nucleosides comprising one or more unnatural bases that are necessary for replication of the plasmid(s) encoding the cytokine gene harboring the codon, (b) the triphosphates of the ribo nucleosides comprising one or more unnatural bases necessary for transcription of (i) the mRNA corresponding to the coding sequence of the cytokine and containing the codon comprising one or more unnatural bases, and (ii) the tRNA containing the anticodon comprising one or more unnatural bases, and (c) the unnatural amino acid(s) to be incorporated in to the polypeptide sequence of the cytokine of interest. The host cells are then maintained under conditions which permit expression of the protein of interest.

The resulting AzK-containing protein that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

The resulting protein comprising the one or more unnatural amino acids, Azk for example, that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

Alternatively, an IL-2 polypeptide comprising an unnatural amino acid(s) is prepared by introducing the nucleic acid constructs described herein comprising the tRNA and aminoacyl tRNA synthetase and comprising a nucleic acid sequence of interest with one or more in-frame orthogonal (stop) codons into a host cell. The host cell is cultured in a medium containing appropriate nutrients, is supplemented with (a) the triphosphates of the deoxyribo nucleosides comprising one or more unnatural bases required for replication of the plasmid(s) encoding the cytokine gene harboring the new codon and anticodon, (b) the triphosphates of the ribo nucleosides required for transcription of the mRNA corresponding to (i) the cytokine sequence containing the codon, and (ii) the orthogonal tRNA containing the anticodon, and (c) the unnatural amino acid(s). The host cells are then maintained under conditions which permit expression of the protein of interest. The unnatural amino acid(s) is incorporated into the polypeptide chain in response to the unnatural codon. For example, one or more unnatural amino acids are incorporated into the IL-2 polypeptide. Alternatively, two or more unnatural amino acids may be incorporated into the IL-2 polypeptide at two or more sites in the protein.

Once the IL-2 polypeptide incorporating the unnatural amino acid(s) has been produced in the host cell it can be extracted therefrom by a variety of techniques known in the art, including enzymatic, chemical and/or osmotic lysis and physical disruption. The IL-2 polypeptide can be purified by standard techniques known in the art such as preparative ion exchange chromatography, hydrophobic chromatography, affinity chromatography, or any other suitable technique known to those of ordinary skill in the art.

Suitable host cells may include bacterial cells (e.g., E. coli, BL21(DE3)), but most suitably host cells are eukaryotic cells, for example insect cells (e.g. Drosophila such as Drosophila melanogaster), yeast cells, nematodes (e.g. C. elegans), mice (e.g. Mus musculus), or mammalian cells (such as Chinese hamster ovary cells (CHO) or COS cells, human 293T cells, HeLa cells, NIH 3T3 cells, and mouse erythroleukemia (MEL) cells) or human cells or other eukaryotic cells. Other suitable host cells are known to those skilled in the art. Suitably, the host cell is a mammalian cell—such as a human cell or an insect cell. In some embodiments, the suitable host cells comprise E. coli.

Other suitable host cells which may be used generally in the embodiments of the invention are those mentioned in the examples section. Vector DNA can be introduced into host cells via conventional transformation or transfection techniques. As used herein, the terms “transformation” and “transfection” are intended to refer to a variety of well-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells are well known in the art.

When creating cell lines, it is generally preferred that stable cell lines are prepared. For stable transfection of mammalian cells for example, it is known that, depending upon the expression vector and transfection technique used, only a small fraction of cells may integrate the foreign DNA into their genome. In order to identify and select these integrants, a gene that encodes a selectable marker (for example, for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector or can be introduced on a separate vector. Cells stably transfected with the introduced nucleic acid molecule can be identified by drug selection (for example, cells that have incorporated the selectable marker gene will survive, while the other cells die).

In one embodiment, the constructs described herein are integrated into the genome of the host cell. An advantage of stable integration is that the uniformity between individual cells or clones is achieved. Another advantage is that selection of the best producers may be carried out. Accordingly, it is desirable to create stable cell lines. In another embodiment, the constructs described herein are transfected into a host cell. An advantage of transfecting the constructs into the host cell is that protein yields may be maximized. In one aspect, there is described a cell comprising the nucleic acid construct or the vector described herein.

### PD-1 Antagonist

In one embodiment, the PD-1 antagonist useful in the treatment, medicaments and uses of the present invention include a monoclonal antibody (mAb), or antigen binding fragment thereof, that specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the treatment method, medicaments and uses of the present invention, are described in U.S. patent nos. U.S. Pat. Nos. 7,488,802, 7,521,051, 8,008,449, 8,354,509, and 8,168,757, and International application publn. nos. WO2004/004771, WO2004/072286, WO2004/056875, US2011/0271358, and WO 2008/156712. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information, Vol. 27, No. 2, pages 161-162 (2013) and that comprises the heavy and light chain amino acid sequences shown in Table 2; nivolumab (BMS-936558), a human IgG4 mAb with the structure described in WHO Drug Information, Vol. 27, No. 1, pages 68-69 (2013) and that comprises the heavy and light chain amino acid sequences shown in Table 2; the humanized antibodies h409A11, h409A16 and h409A17, which are described in WO2008/156712, and AMP-514, which is being developed by MedImmune; cemiplimab; camrelizumab; sintilimab; tislelizumab; and toripalimab. Additional anti-PD-1 antibodies contemplated for use herein include MEDI0680 (U.S. Pat. No. 8,609,089), BGB-A317 (U.S. Patent publ. no. 2015/0079109), INCSHR1210 (SHR-1210) (PCT International application publ. no. WO2015/085847), REGN-2810 (PCT International application publ. no. WO2015/112800), PDR001 (PCT International application publ. no. WO2015/112900), TSR-042 (ANB011) (PCT International application publ. no. WO2014/179664) and STI-1110 (PCT International application publ. no. WO2014/194302).

Examples of mAbs that bind to human PD-L1, and useful in the treatment method, medicaments and uses of the present invention, are described in U.S. Pat. No. 8,383,796. Specific anti-human PD-L1 mAbs useful as the PD-1 antagonist in the treatment method, medicaments and uses of the present invention include BMS-936559, MEDI4736, and MSB0010718C.

In some embodiments, the PD-1 antagonist is pembrolizumab (KEYTRUDA™, Merck & Co., Inc., Rahway, NJ, USA), nivolumab (OPDIVO™, Bristol-Myers Squibb Company, Princeton, NJ, USA), atezolizumab (TECENTRIQ™, Genentech, San Francisco, CA, USA), durvalumab (IMFINZI™, AstraZeneca Pharmaceuticals LP, Wilmington, DE), cemiplimab (LIBTAYO™, Regeneron Pharmaceuticals, Tarrytown, NY, USA) avelumab (BAVENCIO™ Merck KGaA, Darmstadt, Germany) or dostarlimab (JEMPERLI™, GlaxoSmithKline LLC, Philadelphia, PA). In other embodiments, the PD-1 antagonist is pidilizumab (U.S. Pat. No. 7,332,582), AMP-514 (MedImmune LLC, Gaithersburg, MD, USA), PDR001 (U.S. Pat. No. 9,683,048), BGB-A317 (U.S. Pat. No. 8,735,553), or MGA012 (MacroGenics, Rockville, MD).

In one embodiment, the PD-1 antagonist useful in the methods of the invention is an anti-PD-1 antibody that blocks the binding of PD-1 to PD-L1 and PD-L2. In some embodiments of the treatment methods, medicaments and uses of the present invention, the PD-1 antagonist is a monoclonal antibody, or antigen binding fragment thereof, that comprises: (a) a light chain variable region comprising light chain CDR1, CDR2 and CDR3 of SEQ ID NOs: 17, 18 and 19, respectively and (b) a heavy chain variable region comprising heavy chain CDR1, CDR2 and CDR3 of SEQ ID NOs: 22, 23 and 24, respectively.

In other embodiments of the treatment methods, medicaments and uses of the present invention, the PD-1 antagonist is a monoclonal antibody, or antigen binding fragment thereof, that specifically binds to human PD-1 and comprises (a) a heavy chain variable region comprising SEQ ID NO:25 or a variant thereof, and (b) a light chain variable region comprising SEQ ID NO:20 or a variant thereof. A variant of a heavy chain variable region sequence is identical to the reference sequence except having up to six conservative amino acid substitutions in the framework region (i.e., outside of the CDRs). A variant of a light chain variable region sequence is identical to the reference sequence except having up to three conservative amino acid substitutions in the framework region (i.e., outside of the CDRs).

In another embodiment of the treatment methods, medicaments and uses of the present invention, the PD-1 antagonist is a monoclonal antibody that specifically binds to human PD-1 and comprises (a) a heavy chain comprising SEQ ID NO: 26 and (b) a light chain comprising SEQ ID NO:21. In one embodiment, the PD-1 antagonist is an anti-PD-1 antibody that comprises two heavy chains and two light chains, and wherein the heavy and light chains comprise the amino acid sequences in SEQ ID NO:26 and SEQ ID NO:21, respectively.

In all of the above treatment methods, medicaments and uses, the PD-1 antagonist inhibits the binding of PD-L1 to PD-1, and in specific embodiments also inhibits the binding of PD-L2 to PD-1. In some embodiments of the above treatment methods, medicaments and uses, the PD-1 antagonist is a monoclonal antibody, or an antigen binding fragment thereof, that specifically binds to PD-1 or to PD-L1 and blocks the binding of PD-L1 to PD-1.

Table 2 below provides a list of the amino acid sequences of exemplary anti-PD-1 mAbs for use in the treatment method, medicaments and uses of the present invention.

TABLE 2
Exemplary PD-1 Antibody Sequences

| | | |
|---|---|---|
| Antibody | | SEQ |
| Feature | Amino Acid Sequence | ID NO. |

Pembrolizumab Light Chain

| | | |
|---|---|---|
| CDR1 | RASKGVSTSGYSYLH | 17 |
| CDR2 | LASYLES | 18 |
| CDR3 | QHSRDLPLT | 19 |
| Variable | EIVLTQSPATLSLSPGERATLSCRASKGVSTSGYSYLHWY | 20 |
| Region | QQKPGQAPRLLIYLASYLESGVPARFSGSGSGTDFTLTISSL | |
| | EPEDFAVYYCQHSRDLPLTFGGGTKVEIK | |
| Light Chain | EIVLTQSPATLSLSPGERATLSCRASKGVSTSGYSYLHWY | 21 |
| | QQKPGQAPRLLIYLASYLESGVPARFSGSGSGTDFTLTISSL | |
| | EPEDFAVYYCQHSRDLPLTFGGGTKVEIKRTVAAPSVFIFP | |
| | PSDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |

Pembrolizumab Heavy Chain

| | | |
|---|---|---|
| CDR1 | NYYMY | 22 |
| CDR2 | GINPSNGGTNFNEKFKN | 23 |
| CDR3 | RDYRFDMGFDY | 24 |
| Variable | QVQLVQSGVEVKKPGASVKVSCKASGYTFTNYYMYWVR | 25 |
| Region | QAPGQGLEWMGGINPSNGGTNFNEKFKNRVTLTTDSSTT | |
| | TAYMELKSLQFDDTAVYYCARRDYRFDMGFDYWGQGTT | |
| | VTVSS | |
| Heavy | QVQLVQSGVEVKKPGASVKVSCKASGYTFTNYYMYWVR | 26 |
| Chain | QAPGQGLEWMGGINPSNGGTNFNEKFKNRVTLTTDSSTT | |
| | TAYMELKSLQFDDTAVYYCARRDYRFDMGFDYWGQGTT | |
| | VTVSSASTKGPSVFPLAPCSRSTSESTAALGCLVKDYFPEP | |
| | VTVSWNSGALTSGVHTFPAVLQSSGLYSLSSVVTVPSSSL | |
| | GTKTYTCNVDHKPSNTKVDKRVESKYGPPCPPCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLGK | |

TABLE 3
Additional PD-1 Antibodies and Antigen Binding Fragments Useful in the
Formulations, Methods and Uses of the Disclosure.

A. Antibodies and antigen binding fragments comprising light and heavy chain
CDRs of hPD-1.08A in WO2008/156712

| | |
|---|---|
| CDRL1 | RASKSVSTSGFSWLH (SEQ ID NO: 27) |
| CDRL2 | LASNLES (SEQ ID NO: 28) |
| CDRL3 | QHSWQLPLT (SEQ ID NO: 29) |
| CDRH1 | SYYLY (SEQ ID NO: 30) |
| CDRH2 | GVNPSNGGTNFSEKFK (SEQ ID NO: 31) |
| CDRH3 | RDSNYDGGFDY (SEQ ID NO: 32) |

B. Antibodies and antigen binding fragments comprising the mature h109A heavy
chain variable region and one of the mature K09A light chain variable regions in
WO 2008/156712

| | |
|---|---|
| Heavy chain VR | QVQLQQPGAELVKPGTSVKLSCKASGYTFTNYYMY |
| | WVKQRPGQGLEWIGGINPSNGGTNFNGKFKNKATLT |
| | VDSSSSTTYMQLSSLTSEDSAVYYCTRRDYRFDMGF |
| | DYWGQGTTLTVSSAK (SEQ ID NO: 33) |
| Light chain VR | MAPVQLLGLLVLFLPAMRCEIVLTQSPATLSLSPGER |
| | ATLSCRASKGVSTSGYSYLHWYQQKPGQAPRLLIYL |
| | ASYLESGVPARFSGSGSGTDFTLTISSLEPEDFAVYYC |
| | QHSRDLPLTFGGGTKVEIK (SEQ ID NO: 34); |
| | MAPVQLLGLLVLFLPAMRCEIVLTQSPLSLPVTPGEP |
| | ASISCRASKGVSTSGYSYLHWYLQKPGQSPQLLIYLA |
| | SYLESGVPDRFSGSGSGTDFTLKISR VEAEDVGVYYC |
| | QHSRDLPLTFGQGTKLEIK (SEQ ID NO: 35); or |
| | MAPVQLLGLLVLFLPAMRCDIVMTQTPLSLPVTPGEP |
| | ASISCRASKGVSTSGYSYLHWYLQKPGQSPQLLIYLA |
| | SYLESGVPDRFSGSGSGTAFTLKISRVEAEDVGLYYC |
| | QHSRDLPLTFGQGTKLEIK (SEQ ID NO: 36) |

C. Antibodies and antigen binding fragments comprising the mature 409 heavy
chain and one of the mature K09A light chains in WO 2008/156712

| | |
|---|---|
| Heavy chain | MAVLGLLFCLVTFPSCVLSQVQLVQSGVEVKKPGAS |
| | VKVSCKASGYTFTNYYMYWVRQAPGQGLEWMGGI |
| | NPSNGGTNFNEKFKNRVTLTTDSSTTTAYMELKSLQF |
| | DDTAVYYCARRDYRFDMGFDYWGQGTTVTVSSAST |
| | KGPSVFPLAPCSRSTSESTAALGCLVKDYFPEPVTVS |
| | WNSGALTSGVHTFPAVLQSSGLYSLSSVVTVPSSSLG |
| | TKTYTCNVDHKPSNTKVDKRVESKYGPPCPPCPAPEF |
| | LGGPSVFLFPPKPKDTLMISRTPEVTCVVVDVSQEDP |
| | EVQFNWYVDGVEVHNAKTKPREEQFNSTYRVVSVL |
| | TVLHQDWLNGKEYKCKVSNKGLPSSIEKTISKAKGQ |
| | PREPQVYTLPPSQEEMTKNQVSLTCLVKGFYPSDIAV |
| | EWESNGQPENNYKTTPPVLDSDGSFFLYSRLTVDKSR |
| | WQEGNVFSCSVMHEALHNHYTQKSLSLSLGK (SEQ |
| | ID NO: 37) |
| Light chain | MAPVQLLGLLVLFLPAMRCEIVLTQSPATLSLSPGER |
| | ATLSCRASKGVSTSGYSYLHWYQQKPGQAPRLLIYL |
| | ASYLESGVPARFSGSGSGTDFTLTISSLEPEDFAVYYC |
| | QHSRDLPLTFGGGTKVEIKRTVAAPSVFIFPPSDEQLK |
| | SGTASVVCLLNNFYPREAKVQWKVDNALQSGNSQE |
| | SVTEQDSKDSTYSLSSTLTLSKADYEKHKVYACEVT |
| | HQGLSSPVTKSFNRGEC (SEQ ID NO: 38); |
| | MAPVQLLGLLVLFLPAMRCEIVLTQSPLSLPVTPGEP |
| | ASISCRASKGVSTSGYSYLHWYLQKPGQSPQLLIYLA |
| | SYLESGVPDRFSGSGSGTDFTLKISRVEAEDVGVYYC |
| | QHSRDLPLTFGQGTKLEIKRTVAAPSVFIFPPSDEQLK |
| | SGTASVVCLLNNFYPREAKVQWKVDNALQSGNSQE |
| | SVTEQDSKDSTYSLSSTLTLSKADYEKHKVYACEVT |
| | HQGLSSPVTKSFNRGEC (SEQ ID NO: 39); or |
| | MAPVQLLGLLVLFLPAMRCDIVMTQTPLSLPVTPGEP |
| | ASISCRASKGVSTSGYSYLHWYLQKPGQSPQLLIYLA |
| | SYLESGVPDRFSGSGSGTAFTLKISRVEAEDVGLYYC |
| | QHSRDLPLTFGQGTKLEIKRTVAAPSVFIFPPSDEQLK |
| | SGTASVVCLLNNFYPREAKVQWKVDNALQSGNSQE |
| | SVTEQDSKDSTYSLSSTLTLSKADYEKHKVYACEVT |
| | HQGLSSPVTKSFNRGEC (SEQ ID NO: 40) |

In one embodiment, the anti-PD-1 antibody or antigen-binding fragment thereof comprises a heavy chain constant region, e.g. a human constant region, such as g1, g2, g3, or g4 human heavy chain constant region or a variant thereof. In another embodiment, the anti-PD-1 antibody or antigen-binding fragment thereof comprises a light chain constant region, e.g. a human light chain constant region, such as lambda or kappa human light chain region or a variant thereof. By way of example, and not limitation, the human heavy chain constant region can be g4 and the human light chain constant region can be kappa. In an alternative embodiment, the Fc region of the antibody is g4 with a Ser228Pro mutation (Schuurman, J et. al., Mol. Immunol. 38: 1-8, 2001). In some embodiments, different constant domains may be appended to humanized VL and VH regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used. The present invention includes the use of anti-PD-1 antibodies or antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

In another embodiment, the PD-1 antagonist is an antibody or antigen binding protein that has a variable light domain and/or a variable heavy domain with at least 95%, 90%, 85%, 80%, 75% or 50% sequence identity to one of the variable light domains or variable heavy domains described above, and exhibits specific binding to PD-1. In another embodiment of the methods of treatment of the invention, the PD-1 antagonist is an antibody or antigen binding protein comprising variable light and variable heavy domains having up to 1, 2, 3, 4, or 5 or more amino acid substitutions, and exhibits specific binding to PD-1

### Methods of Treatment

In one aspect, provided herein is a method of treating HNSCC in a subject in need thereof, comprising administering to the subject a combination comprising: (a) an IL-2 conjugate as described herein, and (b) pembrolizumab.

In a further aspect, provided herein is a method of treating HNSCC in a subject in need thereof, comprising administering to the subject a combination comprising: (a) an IL-2 conjugate as described herein, (b) pembrolizumab, and (c) cetuximab.

In another aspect, provided herein is a method of treating HNSCC in a subject in need thereof, comprising administering to the subject a combination comprising: (a) an IL-2 conjugate as described herein, (b) pembrolizumab, and (c) an anti-TGFβ antibody.

In one aspect, provided herein is a method of treating HNSCC in a subject in need thereof, comprising administering to the subject a combination comprising: (a) an IL-2 conjugate as described herein, and (b) pembrolizumab, wherein the subject has recurrent and/or metastatic HNSCC. In some embodiments, the method further comprises administering cetuximab to the subject. In other embodiments, the method further comprises administering an anti-transforming growth factor beta (TGFβ) antibody to the subject.

In a further aspect, provided herein is a method of treating HNSCC in a subject in need thereof, comprising: selecting a subject having HNSCC, wherein the subject is selected at least in part on the basis of the subject having recurrent and/or metastatic HNSCC; and administering to the subject a combination comprising: (a) an IL-2 conjugate as described herein, and (b) pembrolizumab. In some embodiments, the method further comprises administering cetuximab to the subject. In other embodiments, the method further comprises administering an anti-transforming growth factor beta (TGFβ) antibody to the subject.

In one aspect, provided herein is a method of treating HNSCC in a subject in need thereof, comprising administering to the subject a combination comprising: (a) an IL-2 conjugate as described herein, and (b) pembrolizumab, wherein the subject has a PD-L1 combined positive score (CPS) greater than or equal to 1. In some embodiments, the method further comprises administering cetuximab to the subject. In other embodiments, the method further comprises administering an anti-transforming growth factor beta (TGFβ) antibody to the subject.

In a further aspect, provided herein is a method of treating HNSCC in a subject in need thereof, comprising: selecting a subject having HNSCC, wherein the subject is selected at least in part on the basis of the subject having a PD-L1 combined positive score (CPS) greater than or equal to 1; and administering to the subject a combination comprising: (a) an IL-2 conjugate as described herein, and (b) pembrolizumab. In some embodiments, the method further comprises administering cetuximab to the subject. In other embodiments, the method further comprises administering an anti-transforming growth factor beta (TGFβ) antibody to the subject.

In a further aspect, provided herein is a method of treating HNSCC in a subject in need thereof, comprising administering to the subject a combination comprising: (a) an IL-2 conjugate as described herein, and (b) pembrolizumab, wherein the HNSCC is recurrent and/or metastatic HNSCC.

In a further aspect, provided herein is a method of treating HNSCC in a subject in need thereof, comprising: selecting a subject having HNSCC, wherein the subject is selected at least in part on the basis of the subject having recurrent and/or metastatic HNSCC; and administering to the subject a combination comprising: (a) an IL-2 conjugate as described herein, and (b) pembrolizumab.

In a further aspect, provided herein is a method of treating HNSCC in a subject in need thereof, comprising administering to the subject a combination comprising: (a) an IL-2 conjugate as described herein, and (b) pembrolizumab, wherein the HNSCC is platinum-refractory HNSCC.

In a further aspect, provided herein is a method of treating HNSCC in a subject in need thereof, comprising: selecting a subject having HNSCC, wherein the subject is selected at least in part on the basis of the subject having platinum-refractory HNSCC; and administering to the subject a combination comprising: (a) an IL-2 conjugate as described herein, and (b) pembrolizumab.

Also provided herein is an IL-2 conjugate as described herein for use in a method disclosed herein of treating HNSCC in a subject in need thereof.

In a further aspect, provided herein is use of an IL-2 conjugate as described herein for the manufacture of a medicament for a method disclosed herein of treating HNSCC in a subject in need thereof.

The embodiments described in the following sections apply to any of the foregoing aspects.

### Cancer Types

In some embodiments, the HNSCC is recurrent and/or metastatic (R/M). In some embodiments, the HNSCC is recurrent. In some embodiments, the HNSCC is metastatic. In some embodiments, the HNSCC is recurrent and metastatic. In some embodiments, the HNSCC is stage III. In some embodiments, the HNSCC is stage IV. In some embodiments, the HNSCC is platinum-refractory HNSCC. In some embodiments, the primary tumor location of the HNSCC is the oropharynx, oral cavity, hypopharynx, or larynx.

### Agents for Combination Therapy

The methods disclosed herein for treatment of HNSCC comprise administering the IL-2 conjugate described herein in combination with one or more additional agents. In some embodiments, the one or more additional agents is pembrolizumab. In some embodiments, the one or more additional agents are pembrolizumab and cetuximab. In some embodiments, the one or more additional agents are pembrolizumab and an anti-TGFβ antibody.

In some embodiments, the anti-TGFβ antibody is a human monoclonal antibody. In some embodiments, the anti-TGFβ antibody neutralizes all isoforms of TGFβ. In some embodiments, the anti-TGFβ antibody has high sequence similarity (e.g., at least 95%, at least 98%, or at least 99% identity) to the amino acid sequence of fresolimumab (GC1008). In some embodiments, the anti-TGFβ antibody comprises the CDRs of fresolimumab. In some embodiments, the anti-TGFβ antibody comprises the VH and VL domains of fresolimumab. In some embodiments, the anti-TGFβ antibody comprises the CDRs of fresolimumab and comprises VH and VL domains that are at least 95%, at least 98%, or at least 99% identical to the VH and VL domains of fresolimumab (e.g., SEQ ID NOs: 4 and 6), respectively. In some embodiments, the anti-TGFβ antibody differs by one or two amino acids from the amino acid sequence of fresolimumab (GC1008), e.g., at a position or positions outside the CDRs. In some embodiments, the anti-TGFβ antibody differs by one amino acid from the amino acid sequence of fresolimumab (GC1008), e.g., at a position outside the CDRs. In some embodiments, the anti-TGFβ antibody differs by one amino acid in the heavy chain (for example, S228P according to the EU numbering scheme) from the amino acid sequence of fresolimumab (GC1008). In some embodiments, the anti-TGFβ antibody differs by two amino acids from the amino acid sequence of fresolimumab (GC1008).

In some embodiments, the light chain sequence of fresolimumab (GC1008) comprises the sequence of SEQ ID NO: 3 (Moulin, A. et al., Protein Sci., (2014), 23 (12), 1698-1707):

(SEQ ID NO: 3)
ETVLTQSPGT LSLSPGERAT LSCRASQSLG SSYLAWYQQK
PGQAPRLLIYGASSRAPGIPDRFSGSGSGTDFTLTISRLEPEDFAVYYCQ
QYADSPITFGQGTRLEIKRTVAAPSVFIFPPSDEQLKSGTASVVCLLNNF
YPREAKVQWKVDNALQSGNSQESVTEQDSKDSTYSLSSTLTLSKADYEKH
KVYACEVTHQGLSSPVTKSF NRGEC

In some embodiments, the VL sequence of fresolimumab (GC1008) comprises amino acids 2-107 of the full-length sequence of SEQ ID NO: 3. In some embodiments, the VL sequence of fresolimumab (GC1008) comprises the sequence of SEQ ID NO: 4:

(SEQ ID NO: 4)
TVLTQSPGTLSLSPGERATLSCRASQSLGSSYLAWYQQKPGQAPRLLIYG
ASSRAPGIPDRFSGSGSGTDFTLTISRLEPEDFAVYYCQQYADSPITFGQ
GTRLEI

In some embodiments, the heavy chain sequence of fresolimumab (GC1008) comprises the sequence of SEQ ID NO: 5 (Moulin, A. et al., Protein Sci., (2014), 23 (12), 1698-1707):

(SEQ ID NO: 5)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRV

In some embodiments, the VH sequence of fresolimumab (GC1008) comprises amino acids 4-119 of the full-length sequence of SEQ ID NO: 5.

In some embodiments, the VH sequence of fresolimumab (GC1008) comprises the sequence of SEQ ID NO: 6:

(SEQ ID NO: 6)
LVQSGAEVKKPGSSVKVSCKASGYTFSSNVISWVRQAPGQGLEWMGGVIP
IVDIANYAQRFKGRVTITADESTSTTYMELSSLRSEDTAVYYCASTLGLV
LDAMDYWGQGTLVTVS

In some embodiments, the anti-TGFβ antibody comprises the light chain CDRs of SEQ ID NO: 3 and the heavy chain CDRs of SEQ ID NO: 5. In some embodiments, the anti-TGFβ antibody comprises the VL sequence of SEQ ID NO: 4 and the VH sequence of SEQ ID NO: 6. In some embodiments, the anti-TGFβ antibody comprises a light chain comprising the sequence of SEQ ID NO: 3 and a heavy chain comprising the sequence of SEQ ID NO: 5. In some embodiments, the anti-TGFβ antibody comprises a light chain comprising the sequence of SEQ ID NO: 3 and a heavy chain comprising the sequence of SEQ ID NO: 5.

In some embodiments, the anti-TGFβ antibody is SAR439459 (see, e.g., Greco, R. et al., OncoImmunology, 2020, 9:1, e1811605).

In some embodiments, the anti-TGFβ antibody comprises a light chain comprising the sequence of SEQ ID NO: 7 and a heavy chain comprising the sequence of SEQ ID NO: 8.

(SEQ ID NO: 7)
ETVLTQSPGTLSLSPGERATLSCRASQSLGSSYLAWYQQKPGQAPRLLIY
GASSRAPGIPDRFSGSGSGTDFTLTISRLEPEDFAVYYCQQYADSPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
(SEQ ID NO: 8)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK.

In some embodiments, the VL sequence of the anti-TGFβ antibody comprises the sequence of SEQ ID NO: 9:

(SEQ ID NO: 9)
ETVLTQSPGTLSLSPGERATLSCRASQSLGSSYLAWYQQKPGQAPRLLIY
GASSRAPGIPDRFSGSGSGTDFTLTISRLEPEDFAVYYCQQYADSPITFG
QGTR

In some embodiments, the LCDR-1 sequence of the anti-TGFβ antibody comprises the sequence of SEQ ID NO: 10:

(SEQ ID NO: 10)
RASQSLGSSYLA.

In some embodiments, the LCDR-2 sequence of the anti-TGFβ antibody comprises the sequence of SEQ ID NO: 11: GASSRAP (SEQ ID NO: 11).

(SEQ ID NO: 11)
GASSRAP.

In some embodiments, the LCDR-3 sequence of the anti-TGFβ antibody comprises the sequence of SEQ ID NO: 12:

(SEQ ID NO: 12)
QQYADSPIT.

In some embodiments, the VH sequence of the anti-TGFβ antibody comprises the sequence of SEQ ID NO: 13:

(SEQ ID NO: 13)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSS.

In some embodiments, the HCDR-1 sequence of the anti-TGFβ antibody comprises the sequence of SEQ ID NO: 14:

(SEQ ID NO: 14)
SNVIS.

In some embodiments, the HCDR-2 sequence of the anti-TGFβ antibody comprises the sequence of SEQ ID NO: 15:

(SEQ ID NO: 15)
GVIPIVDIANYAQRFKG.

In some embodiments, the HCDR-3 sequence of the anti-TGFβ antibody comprises the sequence of SEQ ID NO: 16:

(SEQ ID NO: 16)
TLGLVLDAMDY.

In some embodiments, the sequence of the anti-TGFβ antibody comprises: an HCDR-1 sequence comprising the sequence of SEQ ID NO: 14; an HCDR-2 sequence comprising the sequence of SEQ ID NO: 15; an HCDR-3 sequence comprising the sequence of SEQ ID NO: 16; an LCDR-1 sequence comprising the sequence of SEQ ID NO: 10; an LCDR-2 sequence comprising the sequence of SEQ ID NO: 11; and an LCDR-3 sequence comprising the sequence of SEQ ID NO: 12. In some embodiments, the sequence of the anti-TGFβ antibody comprises: a VH sequence comprising the sequence of SEQ ID NO: 13; and a VL sequence comprising the sequence of SEQ ID NO: 9.

In some embodiments, the sequence of the anti-TGFβ antibody comprises: a VH sequence comprising the sequence of SEQ ID NO: 13; an LCDR-1 sequence comprising the sequence of SEQ ID NO: 10; an LCDR-2 sequence comprising the sequence of SEQ ID NO: 11; and an LCDR-3 sequence comprising the sequence of SEQ ID NO: 12.

In some embodiments, the sequence of the anti-TGFβ antibody comprises: a VL sequence comprising the sequence of SEQ ID NO: 9; an HCDR-1 sequence comprising the sequence of SEQ ID NO: 14; an HCDR-2 sequence comprising the sequence of SEQ ID NO: 15; and an HCDR-3 sequence comprising the sequence of SEQ ID NO: 16.

### Administration

### 1. Route

In some embodiments, the IL-2 conjugate is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, the IL-2 conjugate is administered to the subject by intravenous, subcutaneous, or intramuscular administration. In some embodiments, the IL-2 conjugate is administered to the subject by intravenous administration. In some embodiments, the IL-2 conjugate is administered to the subject by subcutaneous administration. In some embodiments, the IL-2 conjugate is administered to the subject by intramuscular administration.

In some embodiments, pembrolizumab is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, pembrolizumab is administered to the subject by intravenous, subcutaneous, or intramuscular administration. In some embodiments, pembrolizumab is administered to the subject by intravenous administration. In some embodiments, pembrolizumab is administered to the subject by subcutaneous administration. In some embodiments, pembrolizumab is administered to the subject by intramuscular administration.

In some embodiments, the anti-TGFβ antibody is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, the anti-TGFβ antibody is administered to the subject by intravenous, subcutaneous, or intramuscular administration. In some embodiments, the anti-TGFβ antibody is administered to the subject by intravenous administration. In some embodiments, the anti-TGFβ antibody is administered to the subject by subcutaneous administration. In some embodiments, the anti-TGFβ antibody is administered to the subject by intramuscular administration.

In some embodiments, the IL-2 conjugate and pembrolizumab are each administered by intravenous administration. In some embodiments, the IL-2 conjugate, pembrolizumab, and cetuximab are each administered by intravenous administration. In some embodiments, the IL-2 conjugate, pembrolizumab, and the anti-TGFβ antibody are each administered by intravenous administration.

### 2. Schedule

The IL-2 conjugate may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

Pembrolizumab may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

Cetuximab may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

The anti-TGFβ antibody may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

In some embodiments, the IL-2 conjugate and pembrolizumab are administered more than once, e.g., twice, three times, four times, five times, or more. In some embodiments, the IL-2 conjugate, pembrolizumab, and cetuximab are administered more than once, e.g., twice, three times, four times, five times, or more. In some embodiments, the IL-2 conjugate, pembrolizumab, and the anti-TGFβ antibody are administered more than once, e.g., twice, three times, four times, five times, or more. In any of these embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

In some embodiments, the IL-2 conjugate is administered to a subject in need thereof about once every week, about once every two weeks, about once every three weeks, or about once every 4 weeks. In some embodiments, the IL-2 conjugate is administered to a subject in need thereof once every week. In some embodiments, the IL-2 conjugate is administered to a subject in need thereof once every two weeks. In some embodiments, the IL-2 conjugate is administered to a subject in need thereof once every three weeks. In some embodiments, the IL-2 conjugate is administered to a subject in need thereof once every 4 weeks. In some embodiments, the IL-2 conjugate is administered about once every 7, 14, 15, 16, 17, 18, 19, 20, or 21 days.

In some embodiments, pembrolizumab is administered to a subject in need thereof about once every week, about once every two weeks, about once every three weeks, about once every 4 weeks or about once every 6 weeks. In some embodiments, pembrolizumab is administered to a subject in need thereof once every week. In some embodiments, pembrolizumab is administered to a subject in need thereof once every two weeks. In some embodiments, pembrolizumab is administered to a subject in need thereof once every three weeks. In some embodiments, pembrolizumab is administered to a subject in need thereof once every 4 weeks. In some embodiments, pembrolizumab is administered to a subject in need thereof once every six weeks. In some embodiments, pembrolizumab is administered about once every 7, 14, 15, 16, 17, 18, 19, 20, or 21 days.

In some embodiments, cetuximab is administered to a subject in need thereof about once every week, about once every two weeks, about once every three weeks, or about once every 4 weeks. In some embodiments, cetuximab is administered to a subject in need thereof once every week. In some embodiments, cetuximab is administered to a subject in need thereof once every two weeks. In some embodiments, cetuximab is administered to a subject in need thereof once every three weeks. In some embodiments, cetuximab is administered to a subject in need thereof once every 4 weeks. In some embodiments, cetuximab is administered about once every 7, 14, 15, 16, 17, 18, 19, 20, or 21 days.

In some embodiments, the IL-2 conjugate and pembrolizumab are administered to a subject in need thereof about once every week, about once every two weeks, about once every three weeks, about once every 4 weeks, or about once every 6 weeks. In some embodiments, the IL-2 conjugate and pembrolizumab are administered to a subject in need thereof once every week. In some embodiments, the IL-2 conjugate and pembrolizumab are administered to a subject in need thereof once every two weeks. In some embodiments, the IL-2 conjugate and pembrolizumab are administered to a subject in need thereof once every three weeks. In some embodiments, the IL-2 conjugate and pembrolizumab are administered to a subject in need thereof once every 4 weeks. In some embodiments, the IL-2 conjugate and pembrolizumab are administered about once every 7, 14, 15, 16, 17, 18, 19, 20, or 21 days.

In some embodiments, the IL-2 conjugate and cetuximab are administered to a subject in need thereof about once every week, about once every two weeks, about once every three weeks, or about once every 4 weeks. In some embodiments, the IL-2 conjugate and cetuximab are administered to a subject in need thereof once every week. In some embodiments, the IL-2 conjugate and cetuximab are administered to a subject in need thereof once every two weeks. In some embodiments, the IL-2 conjugate and cetuximab are administered to a subject in need thereof once every three weeks. In some embodiments, the IL-2 conjugate and cetuximab are administered to a subject in need thereof once every 4 weeks. In some embodiments, the IL-2 conjugate and cetuximab are administered about once every 7, 14, 15, 16, 17, 18, 19, 20, or 21 days. In some embodiments, the IL-2 conjugate is administered to a subject in need thereof about once every 3 weeks, and cetuximab is administered to the subject about once every week.

In some embodiments, the IL-2 conjugate and the anti-TGFβ antibody are administered to a subject in need thereof about once every week, about once every two weeks, about once every three weeks, or about once every 4 weeks. In some embodiments, the IL-2 conjugate and the anti-TGFβ antibody are administered to a subject in need thereof once every week. In some embodiments, the IL-2 conjugate and the anti-TGFβ antibody are administered to a subject in need thereof once every two weeks. In some embodiments, the IL-2 conjugate and the anti-TGFβ antibody are administered to a subject in need thereof once every three weeks. In some embodiments, the IL-2 conjugate and the anti-TGFβ antibody are administered to a subject in need thereof once every 4 weeks. In some embodiments, the IL-2 conjugate and the anti-TGFβ antibody are administered about once every 7, 14, 15, 16, 17, 18, 19, 20, or 21 days.

In some embodiments, the IL-2 conjugate, pembrolizumab, and cetuximab are administered to a subject in need thereof about once every week, about once every two weeks, about once every three weeks, about once every 4 weeks, or about once every 6 weeks. In some embodiments, the IL-2 conjugate, pembrolizumab, and cetuximab are administered to a subject in need thereof once every week. In some embodiments, the IL-2 conjugate, pembrolizumab, and cetuximab are administered to a subject in need thereof once every two weeks. In some embodiments, the IL-2 conjugate, pembrolizumab, and cetuximab are administered to a subject in need thereof once every three weeks. In some embodiments, the IL-2 conjugate, pembrolizumab, and cetuximab are administered to a subject in need thereof once every 4 weeks. In some embodiments, the IL-2 conjugate, pembrolizumab, and cetuximab are administered about once every 7, 14, 15, 16, 17, 18, 19, 20, or 21 days. In some embodiments, the IL-2 conjugate and pembrolizumab are administered about once every 3 weeks, and cetuximab is administered about once every week.

In some embodiments, the IL-2 conjugate, pembrolizumab, and the anti-TGFβ antibody are administered to a subject in need thereof about once every week, about once every two weeks, about once every three weeks, about once every 4 weeks, or about once every 6 weeks. In some embodiments, the IL-2 conjugate, pembrolizumab, and the anti-TGFβ antibody are administered to a subject in need thereof once every week. In some embodiments, the IL-2 conjugate, pembrolizumab, and the anti-TGFβ antibody are administered to a subject in need thereof once every two weeks. In some embodiments, the IL-2 conjugate, pembrolizumab, and the anti-TGFβ antibody are administered to a subject in need thereof once every three weeks. In some embodiments, the IL-2 conjugate, pembrolizumab, and the anti-TGFβ antibody are administered to a subject in need thereof once every 4 weeks. In some embodiments, the IL-2 conjugate, pembrolizumab, and the anti-TGFβ antibody are administered about once every 7, 14, 15, 16, 17, 18, 19, 20, or 21 days.

In some embodiments, the IL-2 conjugate is administered to the subject separately from the administration of pembrolizumab. In some embodiments, the IL-2 conjugate and pembrolizumab are administered to the subject sequentially. In some embodiments, the IL-2 conjugate is administered to the subject prior to the administration to the subject of pembrolizumab. In some embodiments, the IL-2 conjugate is administered to the subject after the administration to the subject of pembrolizumab. In some embodiments, the IL-2 conjugate and pembrolizumab are administered to the subject simultaneously. In some embodiments, the IL-2 conjugate and pembrolizumab are administered to the subject on the same day. In some embodiments, the IL-2 conjugate and pembrolizumab are administered to the subject on different days.

In some embodiments, the IL-2 conjugate is administered to the subject separately from the administration of cetuximab. In some embodiments, the IL-2 conjugate and cetuximab are administered to the subject sequentially. In some embodiments, the IL-2 conjugate is administered to the subject prior to the administration to the subject of cetuximab. In some embodiments, the IL-2 conjugate is administered to the subject after the administration to the subject of cetuximab. In some embodiments, the IL-2 conjugate and cetuximab are administered to the subject simultaneously. In some embodiments, the IL-2 conjugate and cetuximab are administered to the subject on the same day. In some embodiments, the IL-2 conjugate and cetuximab are administered to the subject on different days.

In some embodiments, the IL-2 conjugate is administered to the subject separately from the administration of the anti-TGFβ antibody. In some embodiments, the IL-2 conjugate and the anti-TGFβ antibody are administered to the subject sequentially. In some embodiments, the IL-2 conjugate is administered to the subject prior to the administration to the subject of the anti-TGFβ antibody. In some embodiments, the IL-2 conjugate is administered to the subject after the administration to the subject of the anti-TGFβ antibody. In some embodiments, the IL-2 conjugate and the anti-TGFβ antibody are administered to the subject simultaneously. In some embodiments, the IL-2 conjugate and the anti-TGFβ antibody are administered to the subject on the same day. In some embodiments, the IL-2 conjugate and the anti-TGFβ antibody are administered to the subject on different days.

In some embodiments, the IL-2 conjugate, pembrolizumab, and cetuximab are each administered to the subject separately. In some embodiments, the IL-2 conjugate, pembrolizumab, and cetuximab are administered to the subject sequentially. In some embodiments, the sequence of administration is (i) pembrolizumab, (ii) cetuximab, and (iii) the IL-2 conjugate. In some embodiments, the sequence of administration is (i) pembrolizumab, (ii) the IL-2 conjugate, and (iii) cetuximab. In some embodiments, the sequence of administration is (i) cetuximab, (ii) pembrolizumab, and (iii) the IL-2 conjugate. In some embodiments, the sequence of administration is (i) cetuximab, (ii) the IL-2 conjugate, and (iii) pembrolizumab. In some embodiments, the sequence of administration is (i) the IL-2 conjugate, (ii) pembrolizumab, and (iii) cetuximab. In some embodiments, the sequence of administration is (i) the IL-2 conjugate, (ii) cetuximab, and (iii) pembrolizumab. In some embodiments, the IL-2 conjugate, pembrolizumab, and cetuximab are administered to the subject simultaneously.

In some embodiments, the IL-2 conjugate, pembrolizumab, and the anti-TGFβ antibody are each administered to the subject separately. In some embodiments, the IL-2 conjugate, pembrolizumab, and cetuximab are administered to the subject sequentially. In some embodiments, the sequence of administration is (i) pembrolizumab, (ii) the anti-TGFβ antibody, and (iii) the IL-2 conjugate. In some embodiments, the sequence of administration is (i) pembrolizumab, (ii) the IL-2 conjugate, and (iii) the anti-TGFβ antibody. In some embodiments, the sequence of administration is (i) the anti-TGFβ antibody, (ii) pembrolizumab, and (iii) the IL-2 conjugate. In some embodiments, the sequence of administration is (i) the anti-TGFβ antibody, (ii) the IL-2 conjugate, and (iii) pembrolizumab. In some embodiments, the sequence of administration is (i) the IL-2 conjugate, (ii) pembrolizumab, and (iii) the anti-TGFβ antibody. In some embodiments, the sequence of administration is (i) the IL-2 conjugate, (ii) the anti-TGFβ antibody, and (iii) pembrolizumab. In some embodiments, the IL-2 conjugate, pembrolizumab, and the anti-TGFβ antibody are administered to the subject simultaneously.

### 3. Dosing

In some instances, the desired doses are conveniently presented in a single dose or as divided doses administered simultaneously (or over a short period of time) or at appropriate intervals, for example as two, three, four or more sub-doses per day.

In some embodiments, the IL-2 conjugate is administered at a dose from about 8 μg/kg to 32 μg/kg. In some embodiments, the IL-2 conjugate is administered at a dose from about 8 g/kg to 24 μg/kg. In some embodiments, the IL-2 conjugate is administered at a dose of about 8 μg/kg. In some embodiments, the IL-2 conjugate is administered at a dose of about 16 μg/kg. In some embodiments, the IL-2 conjugate is administered at a dose of about 24 μg/kg. In some embodiments, the IL-2 conjugate is administered at a dose of about 32 μg/kg. In any of these embodiments, the IL-2 conjugate can be administered at a dose as described herein every 3 weeks.

In some embodiments, the IL-2 conjugate is administered at a dose from about 8 μg/kg to 32 μg/kg in combination with pembrolizumab. In some embodiments, the IL-2 conjugate is administered at a dose from about 8 μg/kg to 24 μg/kg in combination with pembrolizumab. In some embodiments, the IL-2 conjugate is administered at a dose of about 8 μg/kg in combination with pembrolizumab. In some embodiments, the IL-2 conjugate is administered at a dose of about 16 μg/kg in combination with pembrolizumab. In some embodiments, the IL-2 conjugate is administered at a dose of about 24 μg/kg in combination with pembrolizumab. In some embodiments, the IL-2 conjugate is administered at a dose of about 32 μg/kg in combination with pembrolizumab. In any of these embodiments, the IL-2 conjugate can be administered at a dose as described herein every 3 weeks.

In some embodiments, pembrolizumab is administered at a dose of about 200 mg. In some embodiments, pembrolizumab is administered at a dose of about 200 mg every 3 weeks.

In some embodiments, pembrolizumab is administered at a dose of about 400 mg. In some embodiments, pembrolizumab is administered at a dose of about 400 mg every 6 weeks.

In some embodiments, pembrolizumab is administered at a dose of about 2 mg/kg. In some embodiment, pembrolizumab is administered at a dose of about 2 mg/kg every three weeks. In particular embodiments, the patient is a pediatric patient.

In some embodiments, pembrolizumab is administered as a 30 minute (−5 minutes/+10 minutes) intravenous infusion. In one embodiment, the selected dose of pembrolizumab is administered by IV infusion over a time period of between 25 and 40 minutes, or about 30 minutes.

In one aspect, pembrolizumab in included in a pharmaceutical composition with a pharmaceutically acceptable carrier or diluent and may include additional pharmaceutically acceptable excipients.

In some embodiments, cetuximab is administered at a loading dose from about 100 mg/m² to about 500 mg/m² by intravenous infusion. In any of the embodiments described herein, the loading dose of cetuximab is mg/m² of the subject's body surface area. In some embodiments, cetuximab is administered at a loading dose of about 100 mg/m² by intravenous infusion. In some embodiments, cetuximab is administered at a loading dose of about 150 mg/m² by intravenous infusion. In some embodiments, cetuximab is administered at a loading dose of about 200 mg/m² by intravenous infusion. In some embodiments, cetuximab is administered at a loading dose of about 250 mg/m² by intravenous infusion. In some embodiments, cetuximab is administered at a loading dose of about 300 mg/m² by intravenous infusion. In some embodiments, cetuximab is administered at a loading dose of about 350 mg/m² by intravenous infusion. In some embodiments, cetuximab is administered at a loading dose of about 400 mg/m² by intravenous infusion. In some embodiments, cetuximab is administered at a loading dose of about 450 mg/m² by intravenous infusion. In some embodiments, cetuximab is administered at a loading dose of about 500 mg/m² by intravenous infusion. In some embodiments, the initial dose of cetuximab is administered at a loading dose of about 400 mg/m² by intravenous infusion, and all subsequent doses of cetuximab are administered at a loading dose of about 250 mg/m² by intravenous infusion. In any of these embodiments, cetuximab is infused over about 30-240 minutes. In some embodiments, cetuximab is infused over about 30 minutes. In some embodiments, cetuximab is infused over about 60 minutes. In some embodiments, cetuximab is infused over about 90 minutes. In some embodiments, cetuximab is infused over about 120 minutes. In some embodiments, cetuximab is infused over about 150 minutes. In some embodiments, cetuximab is infused over about 180 minutes. In some embodiments, cetuximab is infused over about 210 minutes. In some embodiments, cetuximab is infused over about 240 minutes. In any of these embodiments, cetixumab is administered at an infusion rate of about 1 mg/min to about 10 mg/min, such as 1 mg/min, 2 mg/min, 3 mg/min, 4 mg/min, 5 mg/min, 6 mg/min, 7 mg/min, 8 mg/min, 9 mg/min, or 10 mg/min. In some embodiments, the first dose of cetuximab is administered at a higher loading dose than the dose of subsequent doses of cetuximab. In some embodiments, the infusion time of the first dose of cetuximab is longer than the infusion time of subsequent doses of cetuximab. In some embodiments, cetuximab is administered at a dose as described herein every 3 weeks. In some embodiments, cetuximab is administered at a dose as described herein every 2 weeks. In some embodiments, cetuximab is administered at a dose as described herein every week.

In some embodiments, the anti-TGFβ antibody is administered at a dose of about 15 mg/kg to 25 mg/kg. In some embodiments, the anti-TGFβ antibody is administered at a dose of about 15 mg/kg. In some embodiments, the anti-TGFβ antibody is administered at a dose of about 15.5 mg/kg. In some embodiments, the anti-TGFβ antibody is administered at a dose of about 16 mg/kg. In some embodiments, the anti-TGFβ antibody is administered at a dose of about 16.5 mg/kg. In some embodiments, the anti-TGFβ antibody is administered at a dose of about 17 mg/kg. In some embodiments, the anti-TGFβ antibody is administered at a dose of about 17.5 mg/kg. In some embodiments, the anti-TGFβ antibody is administered at a dose of about 18 mg/kg. In some embodiments, the anti-TGFβ antibody is administered at a dose of about 18.5 mg/kg. In some embodiments, the anti-TGFβ antibody is administered at a dose of about 19 mg/kg. In some embodiments, the anti-TGFβ antibody is administered at a dose of about 19.5 mg/kg. In some embodiments, the anti-TGFβ antibody is administered at a dose of about 20 mg/kg. In some embodiments, the anti-TGFβ antibody is administered at a dose of about 20.5 mg/kg. In some embodiments, the anti-TGFβ antibody is administered at a dose of about 21 mg/kg. In some embodiments, the anti-TGFβ antibody is administered at a dose of about 21.5 mg/kg. In some embodiments, the anti-TGFβ antibody is administered at a dose of about 22 mg/kg. In some embodiments, the anti-TGFβ antibody is administered at a dose of about 22.5 mg/kg. In some embodiments, the anti-TGFβ antibody is administered at a dose of about 23 mg/kg. In some embodiments, the anti-TGFβ antibody is administered at a dose of about 23.5 mg/kg. In some embodiments, the anti-TGFβ antibody is administered at a dose of about 24 mg/kg. In some embodiments, the anti-TGFβ antibody is administered at a dose of about 24.5 mg/kg. In some embodiments, the anti-TGFβ antibody is administered at a dose of about 25 mg/kg. In some embodiments, the anti-TGFβ antibody is administered at a dose as described herein every 3 weeks. In some embodiments, the anti-TGFβ antibody is administered at a dose of about 22.5 mg/kg every 3 weeks.

### 4. Additional Agents/Premedication

In some embodiments, any of the methods described herein further comprises administering an antihistamine. In some embodiments, the antihistamine is cetirizine. In some embodiments, the antihistamine is promethazine. In some embodiments, the antihistamine is dexchlorpheniramine. In some embodiments, the antihistamine is diphenhydramine. In some embodiments, diphenhydramine is administered intravenously at a dose from about 25 to 50 mg.

In some embodiments, any of the methods described herein further comprises administering an analgesic, such as acetaminophen. In some embodiments, acetaminophen is administered orally at a dose from about 650 to 1000 mg.

In some embodiments, any of the methods described herein further comprises administering a serotonin 5-HT₃ receptor antagonist. In some embodiments, the serotonin 5-HT₃ receptor antagonist is granisetron. In some embodiments, the serotonin 5-HT₃ receptor antagonist is dolasetron. In some embodiments, the serotonin 5-HT₃ receptor antagonist is tropisetron. In some embodiments, the serotonin 5-HT₃ receptor antagonist is palonosetron. In some embodiments, the serotonin 5-HT₃ receptor antagonist is ondansetron. In some embodiments, ondansetron is administered intravenously at a dose from about 8 mg to 0.15 mg/kg.

In some embodiments, any of the methods described herein further comprises administering an antihistamine (such as cetirizine, promethazine, dexchlorpheniramine, or diphenhydramine), an analgesic (such as acetaminophen), and/or a serotonin 5-HT₃ receptor antagonist (such as granisetron, dolasetron, tropisetron, palonosetron, or ondansetron). In some embodiments, the method further comprises administering an antihistamine (such as cetirizine, promethazine, dexchlorpheniramine, or diphenhydramine) and an analgesic (such as acetaminophen). In some embodiments, the method further comprising administering an antihistamine (such as cetirizine, promethazine, dexchlorpheniramine, or diphenhydramine) and a serotonin 5-HT₃ receptor antagonist (such as granisetron, dolasetron, tropisetron, palonosetron, or ondansetron). In some embodiments, the method further comprising administering an analgesic (such as acetaminophen) and a serotonin 5-HT₃ receptor antagonist (such as granisetron, dolasetron, tropisetron, palonosetron, or ondansetron). In some embodiments, any of the methods described herein further comprises administering an antihistamine (such as cetirizine, promethazine, dexchlorpheniramine, or diphenhydramine), an analgesic (such as acetaminophen), and a serotonin 5-HT₃ receptor antagonist (such as granisetron, dolasetron, tropisetron, palonosetron, or ondansetron).

In some embodiments, any of the methods described herein further comprises administering a premedication, for example to prevent or reduce the acute effect of infusion-associated reactions (IAR) or flu-like symptoms. In some embodiments, the premedication is administered prior to administering the IL-2 conjugate and/or cetuximab. In some embodiments, the premedication is administered prior to administering the IL-2 conjugate. In some embodiments, the premedication is administered prior to administering cetuximab. In some embodiments, the premedication is administered prior to administering the IL-2 conjugate and cetuximab.

In some embodiments, the premedication for the IL-2 conjugate is different from the premedication for cetuximab. In some embodiments, the premedication for the IL-2 conjugate is the same as the premedication for cetuximab. In some instances where the premedication for the IL-2 conjugate and cetuximab is the same, only a single dose of premedication is administered. In other instances where the premedication for the IL-2 conjugate and cetuximab is the same, multiple doses of premedication are administered. In some embodiments, the premedication is administered for all doses administered of the IL-2 conjugate. In some embodiments, the premedication is administered for the first 1, 2, 3, 4, 5, 6, 7, 8, 9, or 10 doses of the IL-2 conjugate and not for any subsequent doses of the IL-2 conjugate. In some embodiments, the premedication is administered for the first 4 doses of the IL-2 conjugate and not for any subsequent doses of the IL-2 conjugate. In some embodiments, the premedication is administered for all doses administered of cetuximab. In some embodiments, the premedication is administered for the first 1, 2, 3, 4, 5, 6, 7, 8, 9, or 10 doses of cetuximab and not for any subsequent doses of cetuximab. In some embodiments, the premedication is administered for the first dose of cetuximab and not for any subsequent doses of cetuximab.

In some embodiments, any of the methods described herein further comprises administering premedication prior to administering the IL-2 conjugate. In some embodiments, the IL-2 conjugate premedication is an antihistamine, such as cetirizine, promethazine, dexchlorpheniramine, or diphenhydramine. In some embodiments, the antihistamine is diphenhydramine. In some embodiments, diphenhydramine is administered intravenously at a dose from about 25 to 50 mg. In some embodiments, the IL-2 conjugate premedication is a serotonin 5-HT₃ receptor antagonist (such as granisetron, dolasetron, tropisetron, palonosetron, or ondansetron). In some embodiments, the serotonin 5-HT₃ receptor antagonist is ondansetron. In some embodiments, ondansetron is administered intravenously at a dose from about 8 mg to 0.15 mg/kg. In some embodiments, the IL-2 conjugate premedication is an analgesic (such as acetaminophen). In some embodiments, acetaminophen is administered orally at a dose from about 650 to 1000 mg.

In some embodiments, any of the methods described herein further comprises administering premedication prior to administering cetuximab. In some embodiments, the cetuximab premedication is an antihistamine, such as cetirizine, promethazine, dexchlorpheniramine, or diphenhydramine. In some embodiments, the antihistamine is diphenhydramine. In some embodiments, diphenhydramine is administered intravenously at a dose from about 25 to 50 mg. In some embodiments, the cetuximab premedication is a serotonin 5-HT₃ receptor antagonist (such as granisetron, dolasetron, tropisetron, palonosetron, or ondansetron). In some embodiments, the serotonin 5-HT₃ receptor antagonist is ondansetron. In some embodiments, ondansetron is administered intravenously at a dose from about 8 mg to 0.15 mg/kg. In some embodiments, the cetuximab premedication is an analgesic (such as acetaminophen). In some embodiments, acetaminophen is administered orally at a dose from about 650 to 1000 mg.

In some embodiments, any of the methods described herein further comprises administering a first dose of premedication prior to administering the IL-2 conjugate and a second dose of premedication prior to administering cetuximab. In some embodiments, the premedication for the IL-2 conjugate is the same as the premedication for cetuximab. In some embodiments, the premedication for the IL-2 conjugate is different from the premedication for cetuximab. In some embodiments, the premedication is an antihistamine, such as cetirizine, promethazine, dexchlorpheniramine, or diphenhydramine. In some embodiments, the antihistamine is diphenhydramine. In some embodiments, diphenhydramine is administered intravenously at a dose from about 25 to 50 mg. In some embodiments, the premedication is a serotonin 5-HT₃ receptor antagonist (such as granisetron, dolasetron, tropisetron, palonosetron, or ondansetron). In some embodiments, the serotonin 5-HT₃ receptor antagonist is ondansetron. In some embodiments, ondansetron is administered intravenously at a dose from about 8 mg to 0.15 mg/kg. In some embodiments, the premedication is an analgesic (such as acetaminophen). In some embodiments, acetaminophen is administered orally at a dose from about 650 to 1000 mg. In some embodiments, the premedication comprises an antihistamine and a serotonin 5-HT₃ receptor antagonist. In some embodiments, the premedication comprises an antihistamine and an analgesic. In some embodiments, the premedication comprises a serotonin 5-HT₃ receptor antagonist and an analgesic. In some embodiments, the premedication comprises an antihistamine, a serotonin 5-HT₃ receptor antagonist, and an analgesic. In some instances where the premedication for the IL-2 conjugate and cetuximab is the same (such as diphenhydramine), only a single dose of premedication is administered. In other instances where the premedication for the IL-2 conjugate and cetuximab is the same, multiple doses of premedication are administered.

### 5. Dosing Sequence

In some embodiments, the premedication for the IL-2 conjugate and/or cetuximab is as described above and is administered as part of a dosing sequence comprising administering the IL-2 conjugate.

In some embodiments of the methods described herein, the dosing sequence is as follows: (i) pembrolizumab; (ii) premedication for the IL-2 conjugate; and (iii) the IL-2 conjugate. In some embodiments, the dosing sequence is as follows: (i) premedication for the IL-2 conjugate; (ii) the IL-2 conjugate; and (iii) pembrolizumab.

In some embodiments of the methods described herein, the dosing sequence is as follows: (i) pembrolizumab; (ii) premedication for cetuximab; (iii) cetuximab; (iv) premedication for the IL-2 conjugate; and (v) the IL-2 conjugate. In some variations where the premedication for cetuximab is the same as the premedication for the IL-2 conjugate (such as diphenhydramine), administering the premedication for the IL-2 conjugate may be omitted. In some embodiments, the dosing sequence is as follows: (i) pembrolizumab; (ii) premedication for the IL-2 conjugate; (iii) the IL-2 conjugate; (iv) premedication for cetuximab; and (v) cetuximab. In some variations where the premedication for cetuximab is the same as the premedication for the IL-2 conjugate (such as diphenhydramine), administering the premedication for cetuximab may be omitted.

In some embodiments of the methods described herein, the dosing sequence is as follows: (i) premedication for the IL-2 conjugate; (ii) the IL-2 conjugate; (iii) pembrolizumab; (iv) premedication for cetuximab; (v) cetuximab. In some variations where the premedication for cetuximab is the same as the premedication for the IL-2 conjugate (such as diphenhydramine), administering the premedication for cetuximab may be omitted. In some embodiments, the dosing sequence is as follows: (i) premedication for the IL-2 conjugate; (ii) the IL-2 conjugate; (iii) premedication for cetuximab; (iv) cetuximab; and (v) pembrolizumab. In some variations where the premedication for cetuximab is the same as the premedication for the IL-2 conjugate (such as diphenhydramine), administering the premedication for cetuximab may be omitted.

In some embodiments of the methods described herein, the dosing sequence is as follows: (i) premedication for cetuximab; (ii) cetuximab; (iii) pembrolizumab; (iv) premedication for the IL-2 conjugate; and (v) the IL-2 conjugate. In some variations where the premedication for cetuximab is the same as the premedication for the IL-2 conjugate (such as diphenhydramine), administering the premedication for the IL-2 conjugate may be omitted. In some embodiments, the dosing sequence is as follows: (i) premedication for cetuximab; (ii) cetuximab; (ii) premedication for the IL-2 conjugate; (iv) the IL-2 conjugate; and (v) pembrolizumab. In some variations where the premedication for cetuximab is the same as the premedication for the IL-2 conjugate (such as diphenhydramine), administering the premedication for the IL-2 conjugate may be omitted.

In some embodiments of the methods described herein, the dosing sequence is as follows: (i) pembrolizumab; (ii) premedication for the IL-2 conjugate; (iii) the IL-2 conjugate; and (iv) the anti-TGFβ antibody. In some embodiments, the dosing sequence is as follows: (i) premedication for the IL-2 conjugate; (ii) the IL-2 conjugate; (iii) pembrolizumab; and (iv) the anti-TGFβ antibody. In some embodiments, the dosing sequence is as follows: (i) premedication for the IL-2 conjugate; (ii) the IL-2 conjugate; (iii) the anti-TGFβ antibody; and (iv) pembrolizumab.

In some embodiments, the dosing sequence is as follows: (i) the anti-TGFβ antibody; (ii) pembrolizumab; (iii) premedication for the IL-2 conjugate; and (iv) the IL-2 conjugate. In some embodiments, the dosing sequence is as follows: (i) the anti-TGFβ antibody; (ii) premedication for the IL-2 conjugate; (iii) the IL-2 conjugate; and (iv) pembrolizumab.

In some embodiments, the premedication for the IL-2 conjugate is administered about 30-60 minutes prior to administering the IL-2 conjugate, for example, 30-60 minutes prior to the start of the IL-2 conjugate infusion. In some embodiments, the premedication for cetuximab is administered about 30-60 minutes prior to administering cetuximab, for example, 30-60 minutes prior to the start of cetuximab infusion. In some embodiments, the IL-2 conjugate is administered at least 30 minutes after administering pembrolizumab, for example, at least 30 minutes after completion of pembrolizumab infusion. In some embodiments, pembrolizumab is administered at least 30 minutes after administering the IL-2 conjugate, for example, at least 30 minutes after completion of the IL-2 conjugate infusion.

### Subject

In some embodiments, administration of the IL-2 conjugate and the one or more additional agents is to an adult. In some embodiments, the adult is a male. In other embodiments, the adult is a female. In some embodiments, the adult is at least age 18, 19, 20, 25, 30, 35, 40, 45, 50, 55, 60, 65, 70, 75, 80, 85, 90, or 95 years of age.

In some embodiments, the subject has measurable disease (i.e., HNSCC). Measureable disease may be determined by RECIST v1.1. For example, the subject may have at least one measurable lesion per RECIST v1.1. In some embodiments, the subject has histologically or cytologically confirmed diagnosis of recurrent and/or metastatic (R/M) HNSCC that is not amenable to further therapy with curative intent. In some embodiments, the primary tumor location of the HNSCC in the subject is oropharynx, oral cavity, hypopharynx, or larynx. In some embodiments, the primary tumor location is not the nasopharynx. In some embodiments, the subject HPV p16 status for oropharyngeal cancer is known. In some embodiments, the subject has been determined to have Eastern Cooperative Oncology Group (ECOG) performance status of <2, e.g., 0 or 1. In some embodiments, the subject has adequate cardiovascular, hematological, liver, and renal function, as determined by a physician. In some embodiments, the subject has been determined (e.g., by a physician) to have a life expectancy greater than or equal to 12 weeks. In some embodiments, the subject has had prior anti-cancer therapy before administration of the first treatment dose. In some embodiments, the subject has a histologically or cytologically confirmed diagnosis of R/M HNSCC that is considered not amenable to further therapy with curative intent. In some embodiments, if a subject has oropharyngeal cancer, then the subject has a known human papillomavirus p16 status. In some embodiments, the subject does not have a history of allogenic tissue/solid organ transplant. In some embodiments, the subject did not experience an immune-mediated/related toxicity from prior immunooncology therapy of Grade 4 or leading to discontinuation. In some embodiments, the subject does not have ongoing AEs caused by any prior anti-cancer therapy ≥Grade 2. In some embodiments, the subject does not have baseline oxygen saturation (SpO2)≤92% (without oxygen therapy). In some embodiments, the subject has not received prior IL2-based anticancer treatment. In some embodiments, the subject can temporarily (for at least 36 hours) withhold any antihypertensive medications prior to each dose of the IL-2 conjugate. In some embodiments, for methods in which the therapy comprises administering cetuximab, the subject did not receive prior treatment with cetuximab. In some embodiments, the subject does not have electrolyte (magnesium, calcium, and potassium) levels outside of normal ranges. In some embodiments, a subject meets each of the foregoing criteria.

In some embodiments, the subject has a PD-L1 combined positive score (CPS) greater than or equal to 1. In some embodiments, the subject has a PD-L1 combined positive score (CPS) of 1. In some embodiments, the subject has a PD-L1 combined positive score (CPS) greater than 1.

In some embodiments, the subject is treatment-naïve for R/M HNSCC. In some embodiments, the subject was not previously treated with cetuximab (i.e., the patient is treatment-naïve for cetuximab). In some embodiments, the subject was not previously treated with a PD-1/PD-L1-based regimen (i.e., the patient is treatment-naïve for PD-1/PD-L1 therapy).

In some embodiments, the subject was previously treated with a platinum-based regimen. In some embodiments, the subject has platinum-refractory HNSCC. In some embodiments, the subject was previously treated with a PD-1/PD-L1-based regimen. In some embodiments, the subject's previous treatment for HNSCC comprised failure of no more than two regimens. In some embodiments, the subject's previous treatment for HNSCC comprised failure of one regimen. In some embodiments, the subject's previous treatment for HNSCC comprised failure of two regimens. In some embodiments, the subject's previous treatment for HNSCC comprised failure of no more than two regimens, wherein at least one of the failed regimens was a platinum-based regimen. In some embodiments, the subject's previous treatment for HNSCC comprised failure of no more than two regimens, wherein at least one of the failed regimens was a PD-1/PD-L1-based regimen. In some embodiments, the subject's previous treatment for HNSCC comprised failure of a checkpoint-based regimen. In some embodiments, the subject's previous treatment for HNSCC comprised failure of a checkpoint-based regimen and a platinum-based regimen. In some embodiments, the subject's previous treatment for HNSCC comprised failure of two regimens, wherein one of the failed regimens was a PD-1/PD-L1-based regimen, and the other of the failed regimens was a platinum-based regimen. In some embodiments, the subject has platinum-refractory HNSCC and the subject's previous treatment for HNSCC comprised failure of no more than two regimens. In some embodiments, the subject has platinum-refractory HNSCC and the subject's previous treatment for HNSCC comprised failure of one regimen. In some embodiments, the subject has platinum-refractory HNSCC and the subject's previous treatment for HNSCC comprised failure of two regimens. In some embodiments, the subject is a 1L R/M HNSCC subject. In some embodiments, the subject is a 2/3L R/M HNSCC subject.

In some embodiments, the subject has no known hypersensitivity or contraindications to any of the IL-2 conjugates disclosed herein, PEG, pegylated drugs, pembrolizumab, cetuximab, or an anti-TGFβ antibody. In some embodiments, the subject has not received a previous anticancer treatment comprising IL-2. In some embodiments, the subject has not received a previous anticancer treatment comprising cetuximab. In some embodiments, the subject has not received a previous anticancer treatment comprising an agent that blocks the PD-1/PD-L1 pathway. In some embodiments, the subject has not received a previous anticancer treatment comprising pembrolizumab. In some embodiments, the subject has not received a previous anticancer treatment comprising an anti-TGFβ antibody.

In some embodiments, the subject is selected to receive the IL-2 conjugate and pembrolizumab at least in part on the basis of the subject having a PD-L1 combined positive score (CPS) greater than or equal to 1.

In some embodiments, the subject is selected to receive the IL-2 conjugate, pembrolizumab, and cetuximab at least in part on the basis of the subject having a PD-L1 combined positive score (CPS) greater than or equal to 1.

In some embodiments, the subject is selected to receive the IL-2 conjugate, pembrolizumab, and anti-TGFβ antibody at least in part on the basis of the subject having a PD-L1 combined positive score (CPS) greater than or equal to 1.

In some embodiments, the subject does not have an Eastern Cooperative Oncology Group (ECOG) performance status of greater than or equal to 2. In some embodiments, the subject does not have a predicted life expectancy less than or equal to 3 months.

In some embodiments, the subject does not have active brain metastases or leptomeningeal metastases. In some embodiments, the subject was previously treated for brain metastases, has been clinically stable for at least 4 weeks prior to administration of the IL-2 conjugate combination therapy, has no evidence of new or enlarging brain metastases, and has not received steroids for at least 2 weeks prior to administration of the IL-2 conjugate combination therapy. In some embodiments, the subject has asymptomatic brain metastases (i.e., no neurological symptoms, no requirements for corticosteroids, and no lesion greater than 1.5 cm) and receives regular imaging of the brain as a site of disease.

In some embodiments, the subject has no history of allogenic or solid organ transplant.

In some embodiments, the subject does not have treatment-related immune-mediated (or immune-related) adverse events (AEs) from immune-modulatory agents (including, but not limited to anti-PD-1/PD-L1 agents and anti-cytotoxic T lymphocyte associated protein 4 monoclonal antibodies) that caused permanent discontinuation of the agent, or that were Grade 4 in severity.

In some embodiments, the subject's last administration of prior antitumor therapy (chemotherapy, targeted agents, and immunotherapy) or any investigational treatment was not within 28 days or less than 5 times the half-life, whichever is shorter, prior to administration of the IL-2 conjugate combination therapy. In some embodiments, the subject did not have major surgery or local intervention within 28 days of receiving the IL-2 combination therapy.

In some embodiments, the subject does not have comorbidity requiring corticosteroid therapy (>10 mg prednisone/day or equivalent) within 2 weeks of receiving the first dose of the IL-2 conjugate combination therapy. In some embodiments, the subject receives inhaled or topical steroids, provided that they are not for treatment of an autoimmune disorder. In some embodiments, the subject receives a brief course of steroids (e.g., as prophylaxis for imaging studies due to hypersensitivity to contrast agents).

In some embodiments, the subject has not received antibiotics (excluding topical antibiotics) within 14 days of receiving the first dose of the IL-2 conjugate combination therapy. In some embodiments, the subject does not have any serious systemic fungal, bacterial, viral, or other infections that are not controlled or require IV or oral antibiotics.

In some embodiments, the subject has not had a severe or unstable cardiac condition within 6 months of administration of the IL-2 conjugate combination therapy, such as congestive heart failure (New York Heart Association Class III or IV), cardiac bypass surgery or coronary artery stent placement, angioplasty, left ventricular ejection fraction (LVEF) below 50%, unstable angina, medically uncontrolled hypertension (e.g., ≥160 mmHg systolic or ≥100 mmHg diastolic), uncontrolled cardiac arrhythmia requiring medication (≥Grade 2, according to NCI-CTCAE v5.0), or myocardial infarction. In some embodiments, the subject does not have significant valvular heart disease (including valve replacement), vascular malformation, and aneurysm.

In some embodiments, the subject does not have ongoing AEs caused by a prior anticancer therapy ≥Grade 2 (NCI-CTCAE Version 5.0). In some embodiments, the subject has Grade 2 peripheral neuropathy or Grade 2 alopecia.

In some embodiments, the subject has not had active, known, or suspected autoimmune disease that has required systemic treatment (i.e., use of disease modifying agents, corticosteroids, or immunosuppressive drugs) within 2 years of administering the IL-2 conjugate combination therapy. In some embodiments, the subject has received replacement therapy for an autoimmune disease (e.g., thyroxine, insulin, or physiologic corticosteroid replacement therapy for adrenal or pituitary insufficiency, etc). In some embodiments, the subject has had vitiligo, childhood asthma that has resolved, or psoriasis that does not require systemic treatment.

In some embodiments, the subject does not have pneumonitis or interstitial lung disease, or a history of interstitial lung disease or pneumonitis that required oral or IV glucocorticoids to assist with management.

In some embodiments, the subject has not received radiotherapy within 2 weeks of receiving the first dose of the IL-2 conjugate combination therapy. In some embodiments, the subject has recovered from all radiation-related toxicities, does not require corticosteroids, and did not have radiation pneumonitis. In some embodiments, the subject has had a one-week washout for palliative radiation (≤2 weeks of radiotherapy) relating to non-CNS disease.

In some embodiments, the subject did not receive a live-virus vaccination within 28 days of receiving the first dose of the IL-2 conjugate combination therapy.

In some embodiments, the subject is not HIV-infected with a history of Kaposi sarcoma and/or Multicentric Castleman Disease or known uncontrolled infection with HIV. In some embodiments, the subject is HIV-infected and is on anti-retroviral therapy (ART) and has a well-controlled HIV infection/disease defined as: subjects on ART have a CD4+ T-cell count >350 cells/mm³; subjects on ART have achieved and maintained virologic suppression defined as confirmed HIV RNA level below 50 copies/mL or the lower limit of qualification (below the limit of detection) using a locally available assay and for at least 12 weeks; subjects on ART are on a stable regimen, without changes in drugs or dose modification, for at least 4 weeks prior to receiving the first dose of the IL-2 conjugate combination therapy; combination ART regimen does not contain any antiretroviral medications other than abacavir, dolutegravir, emtricitabine, lamivudine, raltegravir, rilpivirine, or tenoforvir.

In some embodiments, the subject does not have known uncontrolled hepatitis B infection, known untreated hepatitis C infection, active tuberculosis, or severe infection requiring parenteral antibiotic treatment. In some embodiments, the subject has positive HBsAg and has started anti-HBV therapy to control HBV infection prior to receiving the first dose of the IL-2 conjugate combination therapy. In some embodiments, the subject has received antiviral therapy for HBV for at least 4 weeks and has an HBV viral load of less than 100 IU/mL prior to receiving the first dose of the IL-2 conjugate combination therapy. In some embodiments, the subject has a viral load under 100 IU/mL and receives active HBV therapy throughout the IL-2 conjugate combination therapy.

In some embodiments, the subject is positive for anti-hepatitis B core antibody HBc, negative for hepatitis B surface antigen (HBsAg), negative or positive for anti-hepatitis B surface antibody (HBs), has an HBV viral load under 100 IU/mL, and does not require HBV anti-viral prophylaxis.

In some embodiments, the subject has past or ongoing HCV infection and has completed treatment at least 1 month prior to receiving the first dose of the IL-2 conjugate combination therapy. In some embodiments, the subject has positive HCV antibody and undetectable HCV RNA and does not receive anti-HCV therapy.

In some embodiments, the subject does not have a known second malignancy either progressing or requiring active treatment within 3 years prior to administering the IL-2 conjugate combination therapy. In some embodiments, the subject has basal cell carcinoma of the skin, squamous cell carcinoma of the skin, or carcinoma in situ (e.g., breast carcinoma, cervical cancer in situ) and has undergone potentially curative therapy.

In some embodiments, the subject does not have underlying cancer predisposition syndromes including, but not limited to, history of hereditary breast and ovarian cancer syndrome, Ferguson-Smith syndrome, multiple self-healing epithelioma, familial adenomatous polyposis, multiple endocrine neoplasia, or Li-Fraumeni syndrome.

In some embodiments, the subject does not have electrolytes (magnesium, calcium, potassium) outside the normal ranges. In some embodiments, the subject does not have baseline SpO₂≤92% (without oxygen therapy).

In some embodiments, the subject has not received prior IL-2 based anticancer treatment. In some embodiments, the subject is able and willing to take premedication. In some embodiments, the subject is not receiving hepatically metabolized narrow therapeutic index drugs (e.g., digoxin, warfarin) without close monitoring. In some embodiments, the subject is receiving anti-hypertensive treatment and the antihypertensive medication is temporarily withheld (for at least 36 hours) prior to receiving each dose of the IL-2 conjugate combination therapy.

In some embodiments, the subject is not being treated with therapeutic doses of anticoagulants or antiplatelet agents (e.g., 1 mg/kg bid of enoxaparin, 300 mg of aspirin daily, 300 mg of clopidogrel daily or equivalent) within 7 days prior to receiving the first dose of the anti-TGFβ antibody. In some embodiments, the subject receives prophylactic treatment of anticoagulants.

In some embodiments, the subject has not received prior treatment with an agent (approved or investigational) that blocks the PD-1/PD-L1 pathway.

In some embodiments, the subject has not received prior treatment with cetuximab unless used locally for the treatment of locally advanced disease, with no progressive disease for at least 4 months from completion of prior cetuximab therapy.

In some embodiments, the subject has not received prior treatment with an anti-TGFβ antibody or with an agent that blocks the TGFβ pathway.

In some embodiments, the subject is not participating in a clinical study concurrently with receiving the IL-2 conjugate combination therapy.

In some embodiments, the subject does not have any or more of the following: absolute neutrophil count <1500/uL (1.5×10⁹/L) (after at least one week off G-CSF); platelets <100×10³ u/L (after at least 3 days without platelet transfusion); hemoglobin <9 g/dL (without packed red blood cell [pRBC] transfusion within prior 2 weeks; subjects can be on stable dose of erythropoietin (≥approximately 3 months); total bilirubin >1.5× upper limit of normal (ULN) unless direct bilirubin ≤ULN (subjects with known Gilbert disease who have serum bilirubin level ≤3×ULN are not excluded); aspartate aminotransferase and/or alanine aminotransferase >2.5×ULN (or >5×ULN for subjects with liver metastases); estimated glomerular filtration rate (eGFR)<50 mL/min/1.73 m² (Modification of Diet in Renal Disease [MDRD] Formula); International Normalized Ratio (INR) or Prothrombin Time (PT) or Activated Partial Thromboplastin Time (aPTT) >1.5×ULN unless the subject is receiving anticoagulant therapy as long as PT or aPTT is within therapeutic range of intended use of anticoagulants.

### Effects of Administration

In some embodiments, administration of the IL-2 conjugate combination therapy as described herein provides a complete response, a partial response, or stable disease.

In some embodiments, following administration of the IL-2 conjugate combination therapy, the subject experiences a response as measured by the Immune-related Response Evaluation Criteria in Solid Tumors (iRECIST). In some embodiments, following administration of the IL-2 conjugate combination therapy, the subject experiences an Objective Response Rate (ORR) according to RECIST version 1.1. In some embodiments, following administration of the IL-2 conjugate combination therapy, the subject experiences Duration of Response (DOR) according to RECIST versions 1.1. In some embodiments, following administration of the IL-2 conjugate combination therapy, the subject experiences Progression-Free Survival (PFS) according to RECIST version 1.1. In some embodiments, following administration of the IL-2 conjugate combination therapy, the subject experiences Overall Survival according to RECIST version 1.1. In some embodiments, following administration of the IL-2 conjugate combination therapy, the subject experiences Time to Response (TTR) according to RECIST version 1.1. In some embodiments, following administration of the IL-2 conjugate combination therapy, the subject experiences Disease Control Rate (DCR) according to RECIST version 1.1. In any of these embodiments, the subject's experience is based on a physician's review of a radiographic image taken of the subject.

In some embodiments, administration of the IL-2 conjugate combination therapy to the subject does not cause vascular leak syndrome in the subject. In some embodiments, administration of the IL-2 conjugate combination therapy to the subject does not cause Grade 2, Grade 3, or Grade 4 vascular leak syndrome in the subject. In some embodiments, administration of the IL-2 conjugate combination therapy to the subject does not cause Grade 2 vascular leak syndrome in the subject. In some embodiments, administration of the IL-2 conjugate combination therapy to the subject does not cause Grade 3 vascular leak syndrome in the subject. In some embodiments, administration of the IL-2 conjugate combination therapy to the subject does not cause Grade 4 vascular leak syndrome in the subject. In some embodiments, administration of the IL-2 conjugate combination therapy to the subject does not cause loss of vascular tone in the subject.

In some embodiments, administration of the IL-2 conjugate combination therapy to the subject does not cause extravasation of plasma proteins and fluid into the extravascular space in the subject.

In some embodiments, administration of the IL-2 conjugate combination therapy to the subject does not cause hypotension and reduced organ perfusion in the subject.

In some embodiments, administration of the IL-2 conjugate combination therapy to the subject does not cause impaired neutrophil function in the subject. In some embodiments, administration of the IL-2 conjugate combination therapy to the subject does not cause reduced chemotaxis in the subject.

In some embodiments, administration of the IL-2 conjugate combination therapy to the subject is not associated with an increased risk of disseminated infection in the subject. In some embodiments, the disseminated infection is sepsis or bacterial endocarditis. In some embodiments, the disseminated infection is sepsis. In some embodiments, the disseminated infection is bacterial endocarditis. In some embodiments, the subject is treated for any preexisting bacterial infections prior to administration of the IL-2 conjugate combination therapy. In some embodiments, the subject is treated with an antibacterial agent selected from oxacillin, nafcillin, ciprofloxacin, and vancomycin prior to administration of the IL-2 conjugate combination therapy.

In some embodiments, administration of the IL-2 conjugate combination therapy to the subject does not exacerbate a pre-existing or initial presentation of an autoimmune disease or an inflammatory disorder in the subject. In some embodiments, the administration of the IL-2 conjugate combination therapy to the subject does not exacerbate a pre-existing or initial presentation of an autoimmune disease in the subject. In some embodiments, the administration of the IL-2 conjugate combination therapy to the subject does not exacerbate a pre-existing or initial presentation of an inflammatory disorder in the subject. In some embodiments, the autoimmune disease or inflammatory disorder in the subject is selected from Crohn's disease, scleroderma, thyroiditis, inflammatory arthritis, diabetes mellitus, oculo-bulbar myasthenia gravis, crescentic IgA glomerulonephritis, cholecystitis, cerebral vasculitis, Stevens-Johnson syndrome and bullous pemphigoid. In some embodiments, the autoimmune disease or inflammatory disorder in the subject is Crohn's disease. In some embodiments, the autoimmune disease or inflammatory disorder in the subject is scleroderma. In some embodiments, the autoimmune disease or inflammatory disorder in the subject is thyroiditis. In some embodiments, the autoimmune disease or inflammatory disorder in the subject is inflammatory arthritis. In some embodiments, the autoimmune disease or inflammatory disorder in the subject is diabetes mellitus. In some embodiments, the autoimmune disease or inflammatory disorder in the subject is oculo-bulbar myasthenia gravis. In some embodiments, the autoimmune disease or inflammatory disorder in the subject is crescentic IgA glomerulonephritis. In some embodiments, the autoimmune disease or inflammatory disorder in the subject is cholecystitis. In some embodiments, the autoimmune disease or inflammatory disorder in the subject is cerebral vasculitis. In some embodiments, the autoimmune disease or inflammatory disorder in the subject is Stevens-Johnson syndrome. In some embodiments, the autoimmune disease or inflammatory disorder in the subject is bullous pemphigoid.

In some embodiments, administration of the IL-2 conjugate combination therapy to the subject does not cause changes in mental status, speech difficulties, cortical blindness, limb or gait ataxia, hallucinations, agitation, obtundation, or coma in the subject. In some embodiments, administration of the IL-2 conjugate combination therapy to the subject does not cause seizures in the subject. In some embodiments, administration of the IL-2 conjugate combination therapy to the subject is not contraindicated in subjects having a known seizure disorder.

In some embodiments, administration of the IL-2 conjugate combination therapy to the subject does not cause capillary leak syndrome in the subject. In some embodiments, administration of the IL-2 conjugate combination therapy to the subject does not cause Grade 2, Grade 3, or Grade 4 capillary leak syndrome in the subject. In some embodiments, administration of the IL-2 conjugate combination therapy to the subject does not cause Grade 2 capillary leak syndrome in the subject. In some embodiments, administration of the IL-2 conjugate combination therapy to the subject does not cause Grade 3 capillary leak syndrome in the subject. In some embodiments, administration of the IL-2 conjugate combination therapy to the subject does not cause Grade 4 capillary leak syndrome in the subject.

In some embodiments, administration of the IL-2 conjugate combination therapy to the subject does not cause a drop in mean arterial blood pressure in the subject following administration. In some embodiments, administration of the IL-2 conjugate combination therapy to the subject does cause hypotension in the subject. In some embodiments, administration of the IL-2 conjugate combination therapy to the subject does not cause the subject to experience a systolic blood pressure below 90 mm Hg or a 20 mm Hg drop from baseline systolic pressure.

In some embodiments, administration of the IL-2 conjugate combination therapy to the subject does not cause edema or impairment of kidney or liver function in the subject.

In some embodiments, administration of the IL-2 conjugate combination therapy to the subject does not cause eosinophilia in the subject. In some embodiments, administration of the IL-2 conjugate combination therapy to the subject does not cause the eosinophil count in the peripheral blood of the subject to exceed 500 per μL. In some embodiments, administration of the IL-2 conjugate combination therapy to the subject does not cause the eosinophil count in the peripheral blood of the subject to exceed 500 μL to 1500 per μL. In some embodiments, administration of the IL-2 conjugate combination therapy to the subject does not cause the eosinophil count in the peripheral blood of the subject to exceed 1500 per L to 5000 per μL. In some embodiments, administration of the IL-2 conjugate combination therapy to the subject does not cause the eosinophil count in the peripheral blood of the subject to exceed 5000 per μL. In some embodiments, administration of the IL-2 conjugate combination therapy to the subject is not contraindicated in subjects on an existing regimen of psychotropic drugs.

In some embodiments, administration of the IL-2 conjugate combination therapy to the subject is not contraindicated in subjects on an existing regimen of nephrotoxic, myelotoxic, cardiotoxic, or hepatotoxic drugs. In some embodiments, administration of the IL-2 conjugate combination therapy to the subject is not contraindicated in subjects on an existing regimen of aminoglycosides, cytotoxic chemotherapy, doxorubicin, methotrexate, or asparaginase. In some embodiments, administration of the IL-2 conjugate combination therapy to the subject is not contraindicated in subjects receiving combination regimens containing antineoplastic agents. In some embodiments, the antineoplastic agent is selected from dacarbazine, cis-platinum, tamoxifen and interferon-alpha.

In some embodiments, administration of the IL-2 conjugate combination therapy to the subject does not cause one or more Grade 4 adverse events in the subject following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis. In some embodiments, administration of the IL-2 conjugate combination therapy to a group of subjects does not cause one or more Grade 4 adverse events in greater than 1% of the subjects following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis.

In some embodiments, administration of the IL-2 conjugate combination therapy to a group of subjects does not cause one or more adverse events in greater than 1% of the subjects following administration, wherein the one or more adverse events is selected from duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; and tracheo-esophageal fistula.

In some embodiments, administration of the IL-2 conjugate combination therapy to a group of subjects does not cause one or more adverse events in greater than 1% of the subjects following administration, wherein the one or more adverse events is selected from malignant hyperthermia; cardiac arrest; myocardial infarction; pulmonary emboli; stroke; intestinal perforation; liver or renal failure; severe depression leading to suicide; pulmonary edema; respiratory arrest; respiratory failure.

In some embodiments, administration of the IL-2 conjugate combination therapy to the subject stimulates CD8+ cells in a subject. In some embodiments, administration of the IL-2 conjugate combination therapy to the subject stimulates NK cells in a subject. Stimulation may comprise an increase in the number of CD8+ cells in the subject, e.g., about 4, 5, 6, or 7 days after administration, or about 1, 2, 3, or 4 weeks after administration. In some embodiments, the CD8+ cells comprise memory CD8+ cells. In some embodiments, the CD8+ cells comprise effector CD8+ cells. Stimulation may comprise an increase in the proportion of CD8+ cells that are Ki67 positive in the subject, e.g., about 4, 5, 6, or 7 days after administration, or about 1, 2, 3, or 4 weeks after administration. Stimulation may comprise an increase in the number of NK cells in the subject, e.g., about 4, 5, 6, or 7 days after administration, or about 1, 2, 3, or 4 weeks after administration.

In some embodiments, CD8+ cells are expanded in the subject following administration of the IL-2 conjugate combination therapy by at least 1.5-fold, such as by at least 1.6-fold, 1.7-fold, 1.8-fold, or 1.9-fold. In some embodiments, NK cells are expanded in the subject following administration of the IL-2 conjugate combination therapy by at least 5-fold, such as by at least 5.5-fold, 6-fold, or 6.5-fold. In some embodiments, eosinophils are expanded in the subject following administration of the IL-2 conjugate combination therapy by no more than about 2-fold, such as no more than about 1.5-fold, 1.4-fold, or 1.3-fold. In some embodiments, CD4+ cells are expanded in the subject following administration of the IL-2 conjugate combination therapy by no more than about 2-fold, such as no more than about 1.8-fold, 1.7-fold, or 1.6-fold. In some embodiments, the expansion of CD8+ cells and/or NK cells in the subject following administration of the IL-2 conjugate combination therapy is greater than the expansion of CD4+ cells and/or eosinophils. In some embodiments, the expansion of CD8+ cells is greater than the expansion of CD4+ cells. In some embodiments, the expansion of NK cells is greater than the expansion of CD4+ cells. In some embodiments, the expansion of CD8+ cells is greater than the expansion of eosinophils. In some embodiments, the expansion of NK cells is greater than the expansion of eosinophils. Fold expansion is determined relative to a baseline value measured before administration of the IL-2 conjugate. In some embodiments, fold expansion is determined at any of the times after administration, such as about 4, 5, 6, or 7 days after administration, or about 1, 2, 3, or 4 weeks after administration.

In some embodiments, administration of the IL-2 conjugate combination therapy to the subject increases the number of peripheral CD8+T and NK cells in the subject without increasing the number of peripheral CD4+ regulatory T cells in the subject. In some embodiments, administration of the IL-2 conjugate combination therapy to the subject increases the number of peripheral CD8+T and NK cells in the subject without increasing the number of peripheral eosinophils in the subject. In some embodiments, administration of the IL-2 conjugate combination therapy to the subject increases the number of peripheral CD8+T and NK cells in the subject without increasing the number of intratumoral CD8+T and NK cells in the subject and without increasing the number of intratumoral CD4+ regulatory T cells in the subject.

In some embodiments, administration of the IL-2 conjugate combination therapy to the subject does not require the availability of an intensive care facility or skilled specialists in cardiopulmonary or intensive care medicine. In some embodiments, administration of the IL-2 conjugate combination therapy to the subject does not require the availability of an intensive care facility or skilled specialists in cardiopulmonary or intensive care medicine. In some embodiments, administration of the IL-2 conjugate combination therapy to the subject does not require the availability of an intensive care facility. In some embodiments, administration of the IL-2 conjugate combination therapy to the subject does not require the availability of skilled specialists in cardiopulmonary or intensive care medicine.

In some embodiments, administration of the IL-2 conjugate combination therapy does not cause dose-limiting toxicity. In some embodiments, administration of the IL-2 conjugate combination therapy does not cause severe cytokine release syndrome. In some embodiments, the IL-2 conjugate does not induce anti-drug antibodies (ADAs), i.e., antibodies against the IL-2 conjugate. In some embodiments, the anti-TGFβ antibody does not induce anti-drug antibodies (ADAs), i.e., antibodies against the anti-TGFβ antibody. In some embodiments, a lack of induction of ADAs is determined by direct immunoassay for antibodies against PEG and/or ELISA for antibodies against the IL-2 conjugate or the anti-TGFβ antibody. An IL-2 conjugate or an anti-TGFβ antibody is considered not to induce ADAs if a measured level of ADAs is statistically indistinguishable from a baseline (pre-treatment) level or from a level in an untreated control.

In some embodiments, administration of the IL-2 conjugate combination therapy improves an ADCC response to the HNSCC. In some embodiments, administration of the IL-2 conjugate combination therapy expands innate and adaptive immune cells while relieving PD-1/PD-L1 mediated immune suppression. In some embodiments, administration of the IL-2 conjugate combination therapy promotes immune activation within the tumor microenvironment. In some embodiments, administration of the IL-2 conjugate combination therapy overcomes or reduces immune evasion mechanisms and boosts anti-cancer T cell immunity. In some embodiments, administration of the IL-2 conjugate combination therapy inhibits the mechanism responsible for resistance of a tumor, for example, TGFβ activity.

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1. Preparation of Pegylated IL-2 Conjugates

An exemplary method with details for preparing IL-2 conjugates described herein is provided in this Example.

IL-2 employed for bioconjugation was expressed as inclusion bodies in E. coli using methods disclosed herein, using: (a) an expression plasmid encoding (i) the protein with the desired amino acid sequence, which gene contains a first unnatural base pair to provide a codon at the desired position at which an unnatural amino acid N6-((2-azidoethoxy)-carbonyl)-L-lysine (AzK) was incorporated and (ii) a tRNA derived from M. mazei Pyl, which gene comprises a second unnatural nucleotide to provide a matching anticodon in place of its native sequence; (b) a plasmid encoding a M. barkeri derived pyrrolysyl-tRNA synthetase (Mb PylRS), (c) N6-((2-azidoethoxy)-carbonyl)-L-lysine (AzK); and (d) a truncated variant of nucleotide triphosphate transporter PtNTT2 in which the first 65 amino acid residues of the full-length protein were deleted. The double-stranded oligonucleotide that encodes the amino acid sequence of the desired IL-2 variant contained a codon AXC as codon 64 of the sequence that encodes the protein having SEQ ID NO: 1 in which P64 is replaced with an unnatural amino acid described herein. The plasmid encoding an orthogonal tRNA gene from M. mazei comprised an AXC-matching anticodon GYT in place of its native sequence, wherein Y is an unnatural nucleotide as disclosed herein. X and Y were selected from unnatural nucleotides dTPT3 and dNaM as disclosed herein. The expressed protein was extracted from inclusion bodies and re-folded using standard procedures before site-specifically pegylating the AzK-containing IL-2 product using DBCO-mediated copper-free click chemistry to attach stable, covalent mPEG moieties to the AzK. Exemplary reactions are shown in Schemes 1 and 2 (wherein n indicates the number of repeating PEG units). The reaction of the AzK moiety with the DBCO alkynyl moiety may afford one regioisomeric product or a mixture of regioisomeric products.

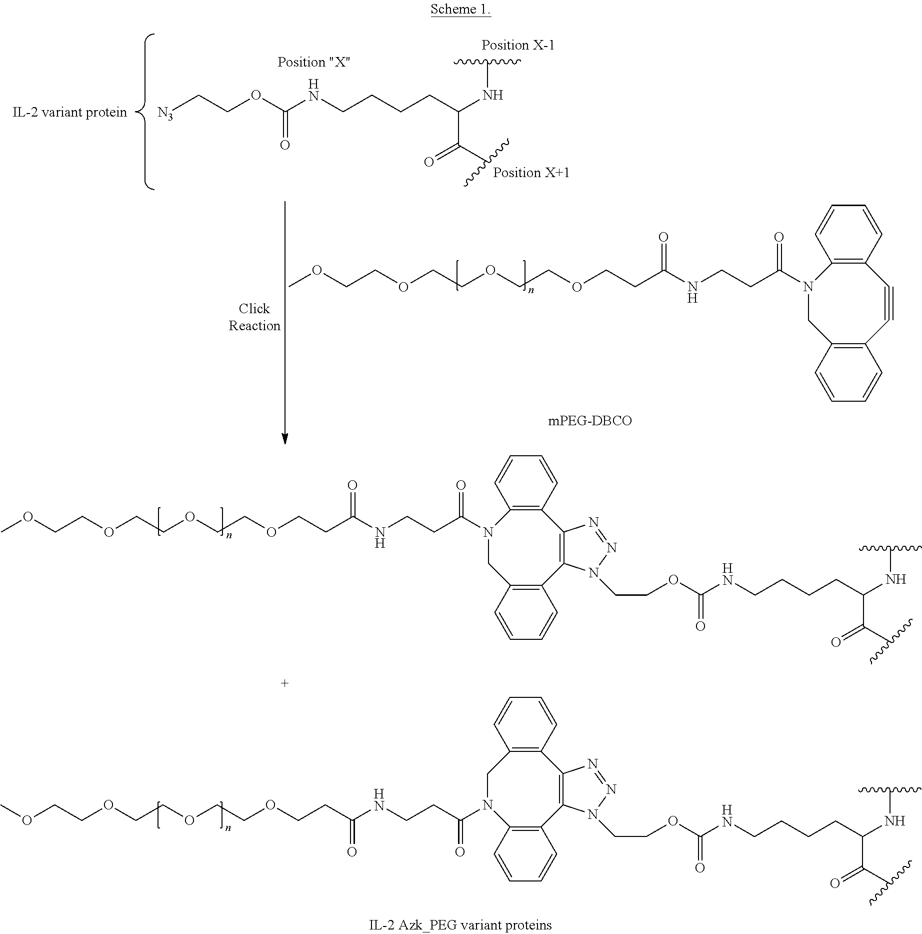

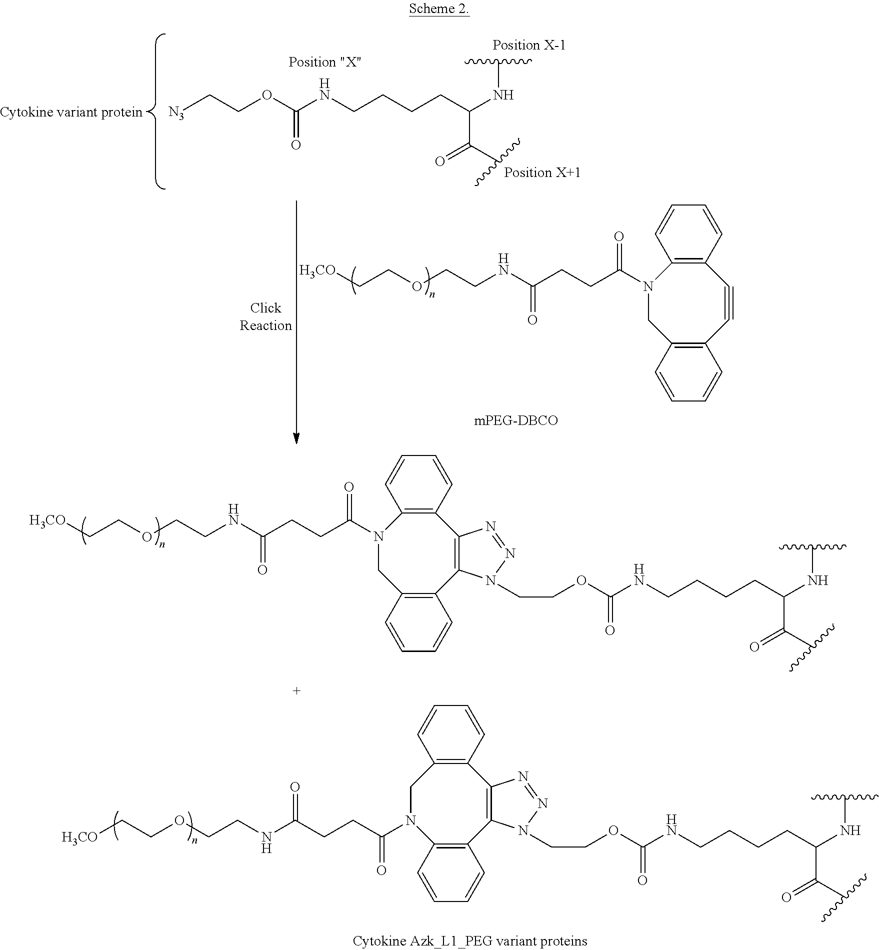

### Example 2. Clinical Study of Biomarker Effects Following IL-2 Conjugate and Pembrolizumab Administration

A study was performed to characterize immunological effects of in vivo administration of an IL-2 conjugate described herein in combination with pembrolizumab. The IL-2 conjugate comprised SEQ ID NO: 2, wherein position 64 is AzK_L1_PEG30kD, where AzK_L1_PEG30kD is defined as a structure of Formula (IV) or Formula (V), or a mixture of Formula (IV) and Formula (V), and a 30 kDa, linear mPEG chain. This IL-2 conjugate can also be described as an IL-2 conjugate comprising SEQ ID NO: 1, wherein position 64 is replaced by the structure of Formula (IV) or Formula (V), or a mixture of Formula (IV) and Formula (V), and a 30 kDa, linear mPEG chain. The IL-2 conjugate can also be described as an IL-2 conjugate comprising SEQ ID NO: 1, wherein position 64 is replaced by the structure of Formula (XII) or Formula (XIII), or a mixture of Formula (XII) and Formula (XIII), and a 30 kDa, linear mPEG chain. The compound was prepared as described in Example 1, i.e., using methods wherein a protein was first prepared having SEQ ID NO: 1 in which the proline at position 64 was replaced by N6-((2-azidoethoxy)-carbonyl)-L-lysine AzK. The AzK-containing protein was then allowed to react under click chemistry conditions with DBCO comprising a methoxy, linear PEG group having an average molecular weight of 30 kDa, followed by purification and formulation employing standard procedures.

The IL-2 conjugate and pembrolizumab were administered via IV infusion for 30 minutes every 3 weeks [Q3W]. Effects on the following biomarkers were analyzed as surrogate predictors of safety and/or efficacy:
Eosinophilia (elevated peripheral eosinophil count): Cell surrogate marker for IL-2-induced proliferation of cells (eosinophils) linked to vascular leak syndrome (VLS);
Interleukin 5 (IL-5): Cytokine surrogate marker for IL-2 induced activation of type 2 innate lymphoid cells and release of this chemoattractant that leads to eosinophilia and potentially VLS;
Interleukin 6 (IL-6): Cytokine surrogate marker for IL-2 induced cytokine release syndrome (CRS); and
Interferon γ (IFN-γ): Cytokine surrogate marker for IL-2 induced activation of CD8+ cytotoxic T lymphocytes and NK cells.

Effects on the cell counts of the following biomarkers were analyzed as surrogate predictors of anti-tumor immune activity:
Peripheral CD8+ Effector Cells: Marker for IL-2-induced proliferation of these target cells in the periphery that upon infiltration become a surrogate marker of inducing a potentially latent therapeutic response;
Peripheral CD8+ Memory Cells: Marker for IL-2-induced proliferation of these target cells in the periphery that upon infiltration become a surrogate marker of inducing a potentially durable latent therapeutic and maintenance of the memory population;
Peripheral NK Cells: Marker for IL-2-induced proliferation of these target cells in the periphery that upon infiltration become a surrogate marker of inducing a potentially rapid therapeutic response; and
Peripheral CD4+ Regulatory Cells: Marker for IL-2-induced proliferation of these target cells in the periphery that upon infiltration become a surrogate marker of inducing an immunosuppressive TME and offsetting of an effector-based therapeutic effect.

Subjects were human males or females aged ≥18 years at screening. All subjects had been previously treated with an anti-cancer therapy and met at least one of the following: Treatment related toxicity resolved to grade 0 or 1 (alopecia excepted) according to NCI CTCAE v5.0; or Treatment related toxicity resolved to at least grade 2 according to NCI CTCAE v5.0 with prior approval of the Medical Monitor. The most common tumors included cervical cancer, head and neck squamous cell carcinoma, basal cell carcinoma, melanoma and non-small cell lung cancer.

Subjects also met the following criteria: Provided informed consent. Eastern Cooperative Oncology Group (ECOG) performance status of 0 or 1. Life expectancy greater than or equal to 12 weeks as determined by the Investigator. Histologically or cytologically confirmed diagnosis of advanced and/or metastatic solid tumors. Subjects with advanced or metastatic solid tumors who have refused standard of care; or for whom no reasonable standard of care exists that would confer clinical benefit; or for whom standard therapy is intolerable, not effective, or not accessible. Measurable disease per RECIST v1.1. Adequate laboratory parameters including: Absolute lymphocyte count ≥0.5 times lower limit of normal; Platelet count ≥100×10⁹/L; Hemoglobin ≥9.0 g/dL (absence of growth factors or transfusions within 2 weeks; 1-week washout for ESA and CSF administration is sufficient); Absolute neutrophil count ≥1.5×10⁹/L (absence of growth factors within 2 weeks); Prothrombin time (PT) and partial thromboplastin time (PTT)≤1.5 times upper limit of normal (ULN); Aspartate aminotransferase (AST) and alanine aminotransferase (ALT)≤2.5 times ULN except if liver metastases are present may be ≤5 times ULN; Total bilirubin ≤1.5×ULN. Premenopausal women and women less than 12 months after menopause had a negative serum pregnancy test within 7 days prior to initiating study treatment.

Subjects ranged in age from 29 to 74 with a mean age 55.0 and a median age of 59.0. All subjects had metastatic disease. 29 subjects were male and 9 were female. 4 subjects were Hispanic or Latino, 32 were not Hispanic or Latino and 2 were not reported. 26 participants were White, 4 were Black or African American, 5 were Asian, 1 was American Indian or Alaska Native, 1 was Other and 1 was not reported. 17 subjects had an ECOG score of 0 and 21 had an ECOG score of 1. Prior lines of systemic therapies were as follows: 5 subjects had 1 line; 12 subjects had 2 lines; 7 subjects had 3 lines; 7 subjects had 4 lines; and 5 subjects had 5+ lines. Primary tumor types included 4 colorectal cancer (CRC), 4 melanoma, 4 sarcoma, 1 prostate, 1 non-small cell lung carcinoma (NSCLC), 1 HNSCC, and 12 other.

Cohorts Treated with 8 μg/Kg and 16 μg/Kg Doses

Q3W dosing. 10 adults (6 [60%] male, 4 [40%] female, 9 [90%] Caucasian) having advanced or metastatic solid tumors and whose age ranged from 42-70 years received a) the IL-2 conjugate at an 8 μg/kg dose IV Q3W or 16 μg/kg dose IV Q3W and b) pembrolizumab at a dose of 200 mg IV Q3W sequentially for at least one cycle. Here and throughout Example 2, drug mass per kg subject (e.g., 8 μg/kg) refers to IL-2 mass exclusive of PEG and linker mass. The results below are for subjects receiving an 8 μg/kg dose IV Q3W and pembrolizumab (4 subjects) or 16 μg/kg dose IV Q3W and pembrolizumab (6 subjects), who received treatment for 2-19 cycles.

Two subjects who received 8 μg/kg IL-2 conjugate and pembrolizumab had confirmed partial responses (PRs; 1 PD-1-naïve basal cell carcinoma, 1 head and neck squamous cell carcinoma, who had received prior anti-PD-1) ongoing for 9+ months. One subject (non-small cell lung cancer) who received 16 μg/kg IL-2 conjugate and pembrolizumab had disease stabilization for about 6 months. Six subjects had disease progression (at the 6-week assessment); one subject had initial disease stabilization (at the 6 week assessment; followed by progressive disease). The four subjects receiving 8 μg/kg IL-2 conjugate and pembrolizumab had increased post-dose CD8+Ki67 expression levels (15%-70%).

One 59 year old male with head and neck squamous cell carcinoma receiving 8 μg/kg IL-2 conjugate and pembrolizumab received 18 cycles (with therapy ongoing) and had a confirmed partial response (about 38% decrease in tumor volume after 8 cycles; about 45% decrease after 11 cycles; about 81% decrease at 18 cycles). This subject had previously received 4 lines of systemic therapy including 2 anti-PD1 treatments; the best response to an anti-PD-1 treatment had been stable disease.

One 50 year old male with basal cell carcinoma receiving 8 μg/kg IL-2 conjugate and pembrolizumab received 17 cycles and had a confirmed partial response (50% decrease after 2 cycles, and 80% decrease after 8 cycles). This subject had previously undergone surgeries and radiation therapy.

The maximal tumor responses in other patients with immune sensitive tumors were found to be melanoma (23% and 11% growth), basal cell carcinoma (4% growth), and non-small cell lung cancer (18% reduction).

The peak peripheral expansion of CD8+T effector cells averaged 2.06-fold above baseline in subjects receiving 8 μg/kg IL-2 conjugate and pembrolizumab. All four subjects had post-dose NK Cell Ki67 expression levels of nearly 100 percent. The subjects had post-dose peak peripheral expansion of NK cells that averaged 6.73-fold above baseline at day 3. The peak peripheral expansion of CD8+T effector cells averaged 3.71-fold above baseline in subjects receiving 16 μg/kg IL-2 conjugate and pembrolizumab.

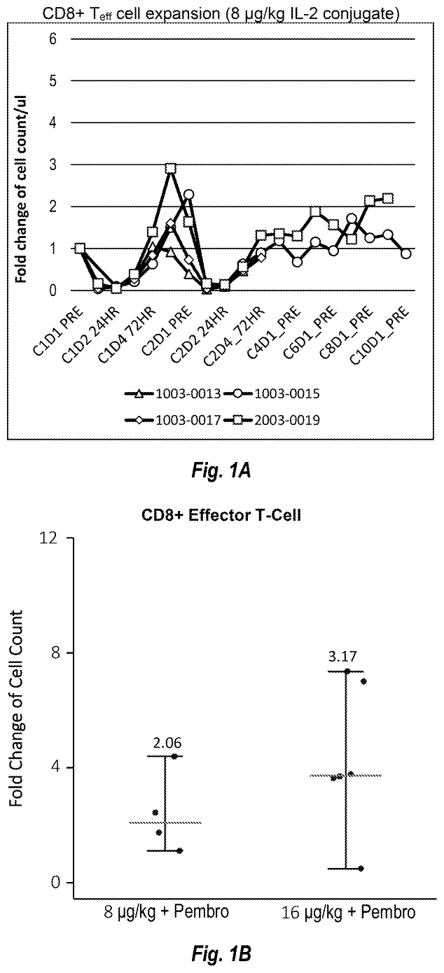

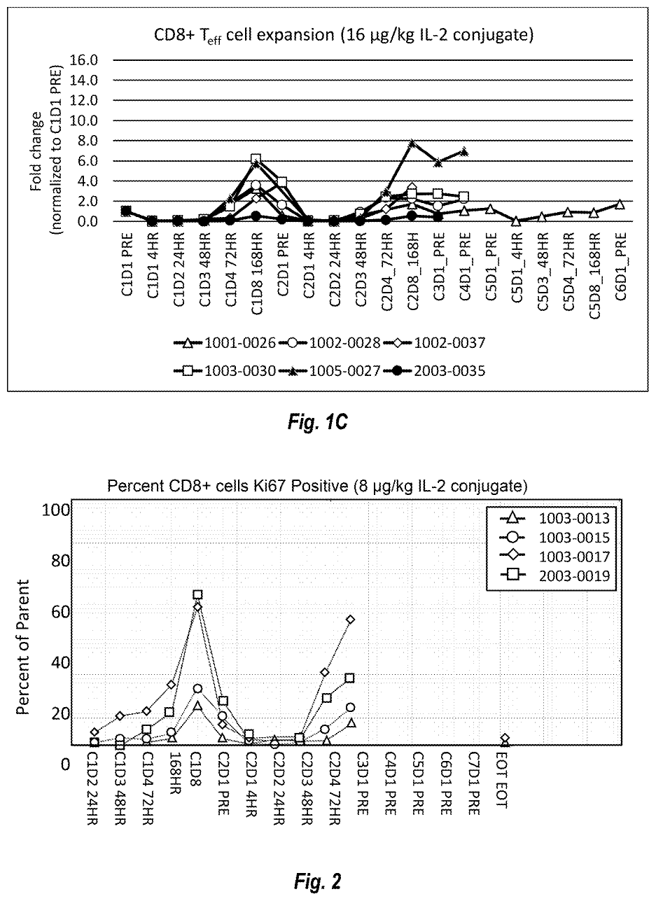

Efficacy biomarkers. Peripheral CD8+T_{eff} cell counts were measured (FIGS. 1A-C). Prolonged CD8+ expansion over baseline (e.g., greater than or equal to 1.5-fold change) was observed at 3 weeks after the previous dose in some subjects. The percentage of CD8+ T_{eff} cells expressing Ki67 was also measured (FIG. 2).

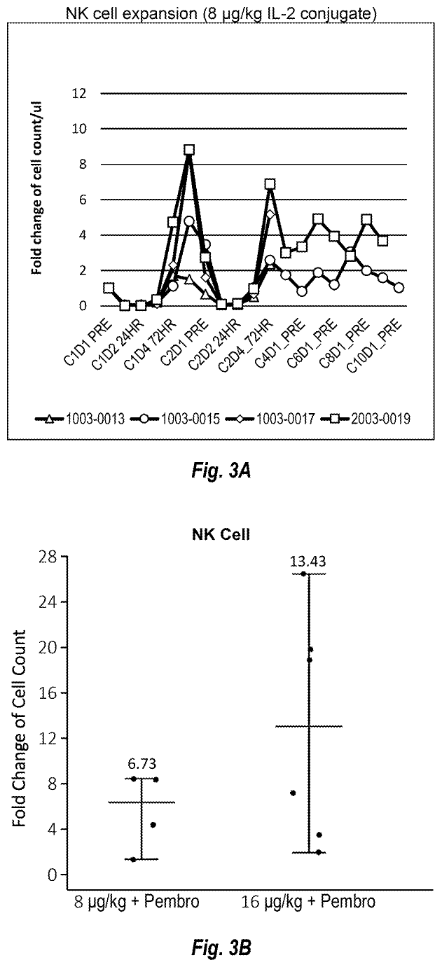

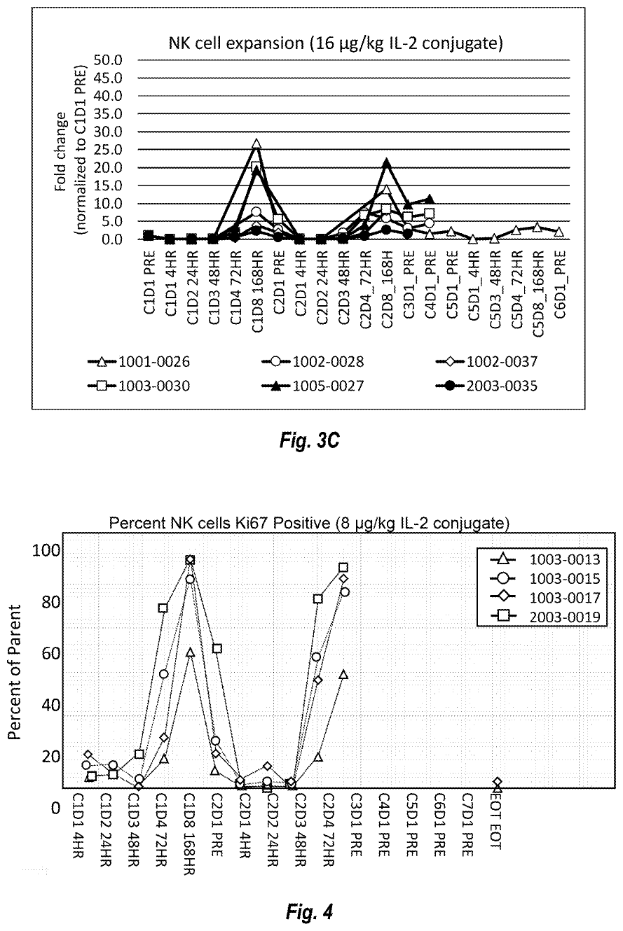

Peripheral NK cell counts are shown in FIGS. 3A-C. Prolonged NK cell expansion over baseline (e.g., greater than or equal to 2-fold change) was observed at 3 weeks after the previous dose in some subjects. The percentage of NK cells expressing Ki67 was also measured (FIG. 4).

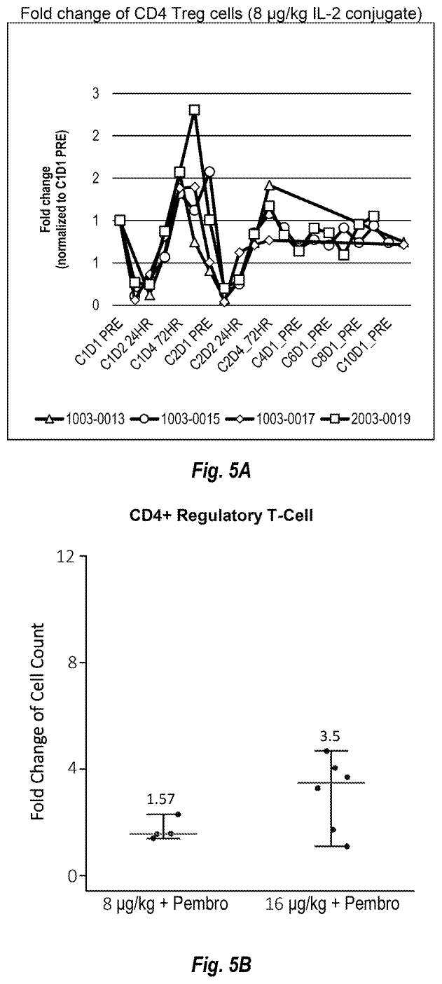

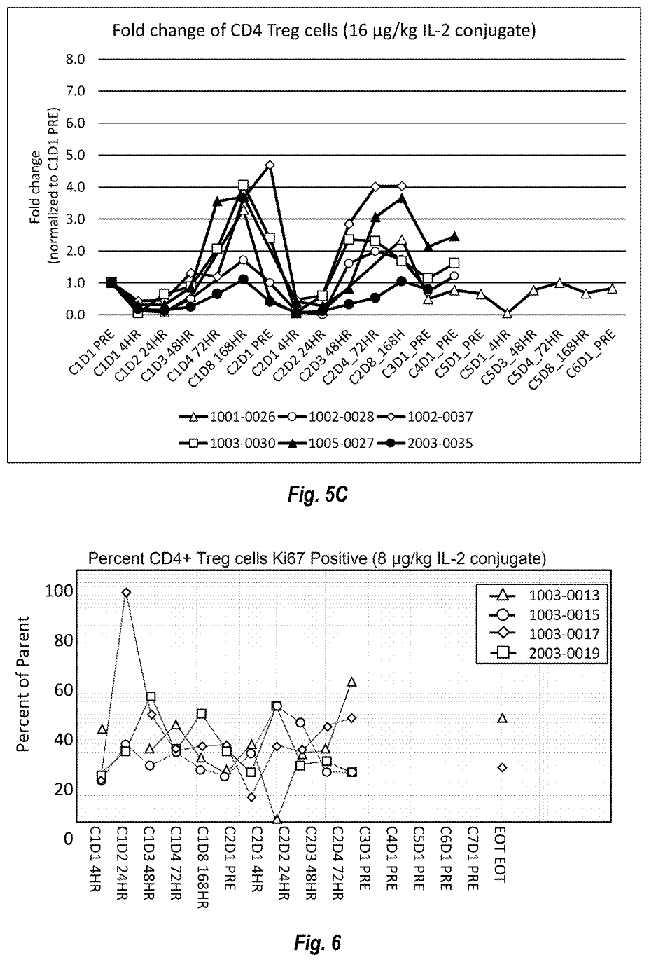

Peripheral CD4+T_{reg} counts are shown in FIGS. 5A-C. The percentage of CD4+T_{reg} cells expressing Ki67 was also measured (FIG. 6).

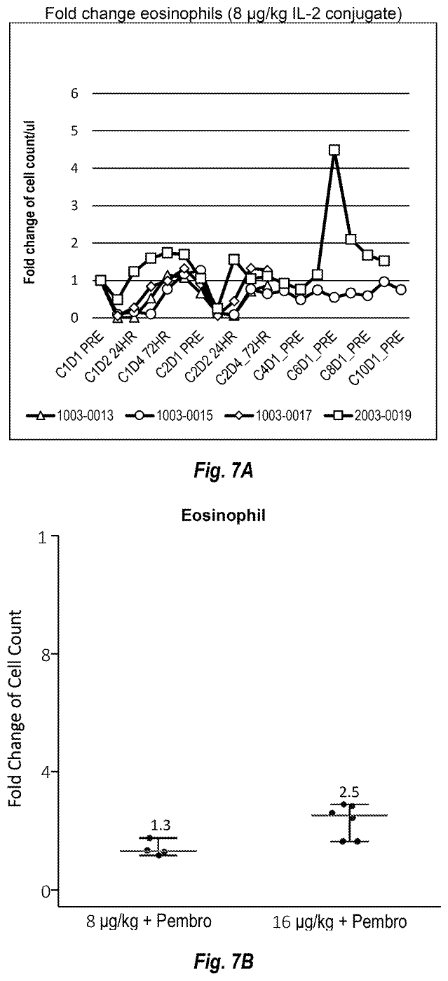

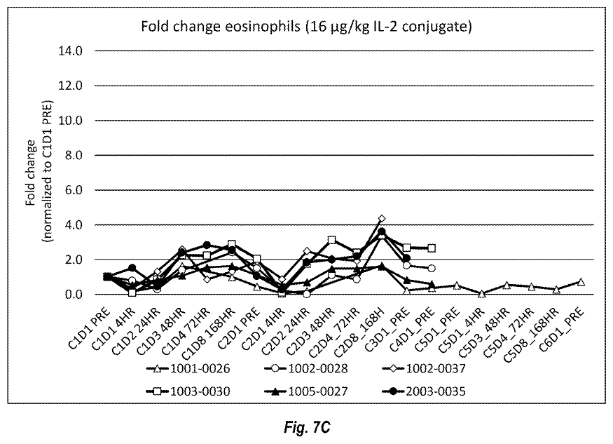

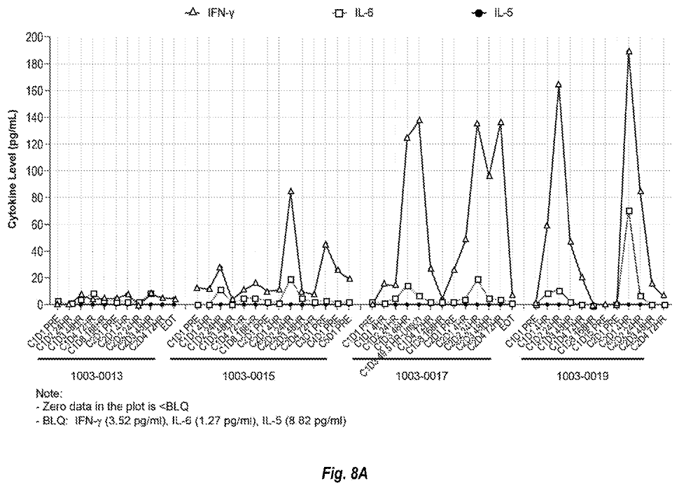

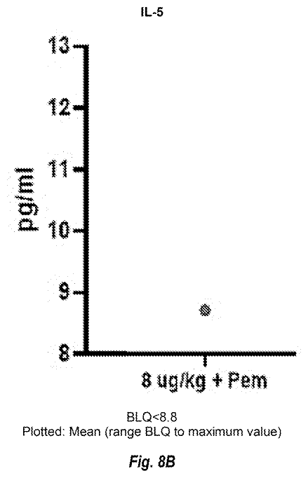

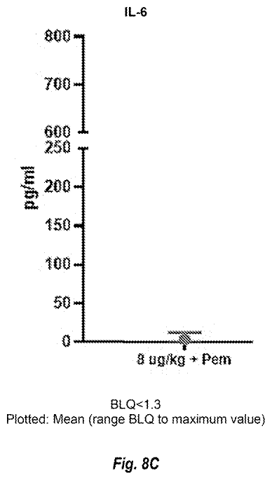

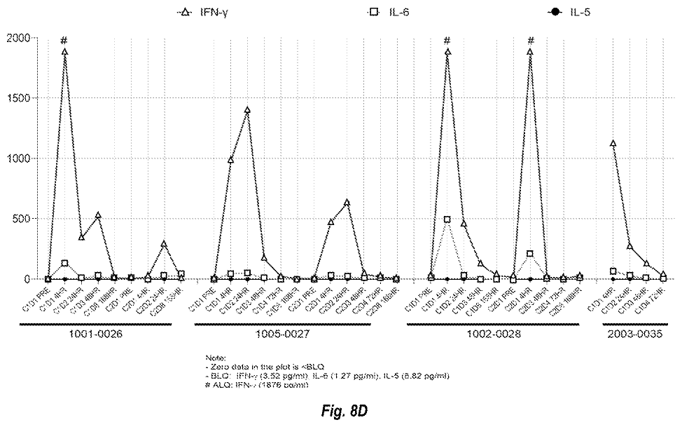

Eosinophil counts were measured (FIGS. 7A-C). The measured values were consistently below the range of 2328-15958 eosinophils/L in patients with IL-2 induced eosinophilia as reported in Pisani et al., Blood 1991 Sep. 15; 78(6):1538-44. Levels of IFN-γ, IL-5, and IL-6 were also measured (FIGS. 8A-D). The measured values show that IFN-γ was induced, but low amounts of IL-5 and IL-6, cytokines associated with VLS and CRS, respectively, were induced.

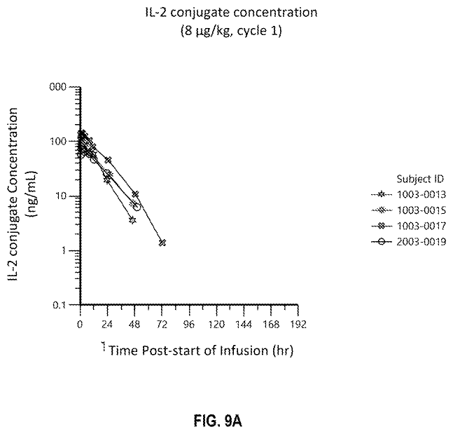

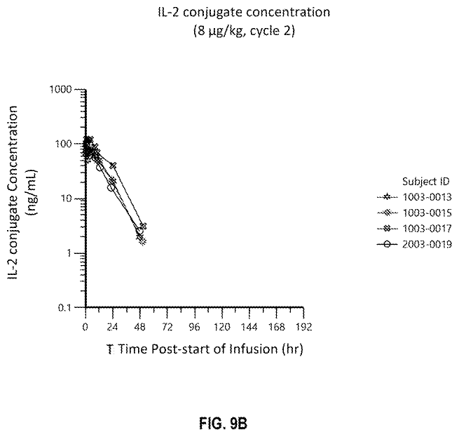

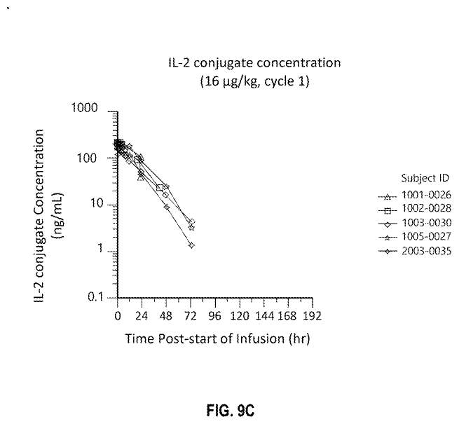

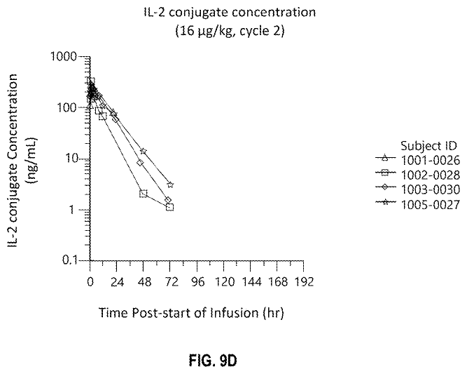

Mean concentrations of the IL-2 conjugate, administered at a dose of 8 μg/kg, after 1 and 2 cycles are shown in FIG. 9A and FIG. 9B, respectively. Mean concentrations of the IL-2 conjugate, administered at a dose of 16 μg/kg, after 1 and 2 cycles are shown in FIG. 9C and FIG. 9D, respectively.

Anti-drug Antibodies (ADAs). Samples from treated subjects were assayed after each dose cycle for anti-drug antibodies (ADAs). Anti-polyethylene glycol autoantibodies were detected by direct immunoassays (detection limit: 36 ng/mL). A bridging MesoScale Discovery ELISA was performed with a labeled form of the IL-2 conjugate, having a detection limit of 4.66 ng/mL. Additionally, a cell-based assay for neutralizing antibodies against the IL-2 conjugate was performed using the CTLL-2 cell line, with STAT5 phosphorylation as the readout (detection limit: 6.3 μg/mL).

Samples were collected and analyzed after each dose cycle from four subjects where 2 patients received 2 cycles and the other two patients received 10 or 11 cycles. An assay-specific cut point was determined during assay qualification as a signal to negative ratio of 1.09 or higher for the IL-2 conjugate ADA assay and 2.08 for the PEG ADA assay. Samples that gave positive or inconclusive results in the IL-2 conjugate assay were subjected to confirmatory testing in which samples and controls were assayed in the presence and absence of confirmatory buffer (10 μg/mL IL-2 conjugate in blocking solution). Samples that gave positive or inconclusive results in the PEG assay were subjected to confirmatory testing in which samples and controls were assayed in the presence and absence of confirmatory buffer (10 μg/mL IL-2 conjugate in 6% horse serum). Samples will be considered “confirmed” if their absorbance signal is inhibited by equal to or greater than an assay-specific cut point determined during assay qualification (14.5% for the IL-2 conjugate or 42.4% for PEG) in the detection step. No confirmed ADA against the IL-2 conjugate or PEG were detected (data not shown).

Summary of Results; Discussion. All subjects had elevated post-dose CD8+Ki67 expression levels (FIG. 2), with peripheral expansion of CD8+T effector (Teff) cells averaging 1.95-fold above baseline. All 4 subjects also had elevated post-dose NK cell Ki67 expression levels (FIG. 4), with peripheral expansion of NK cells averaging 6.73-fold above baseline at day 3. There were no meaningful elevations in IL-5 and IL-6 levels.

An AE was any untoward medical occurrence in a clinical investigation subject administered a pharmaceutical product, regardless of causal attribution. Dose-limiting toxicities were defined as an AE occurring within Day 1 through Day 29 (inclusive)±1 day of a treatment cycle that was not clearly or incontrovertibly solely related to an extraneous cause and that met at least one of the following criteria:
Grade 3 neutropenia (absolute neutrophil count <1000/mm³>500/mm³) lasting ≥7 days, or Grade 4 neutropenia of any duration
Grade 3+ febrile neutropenia
Grade 4+ thrombocytopenia (platelet count <25,000/mm³)
Grade 3+ thrombocytopenia (platelet count <50,000-25,000/mm³) lasting ≥5 days, or associated with clinically significant bleeding or requiring platelet transfusion
Failure to meet recovery criteria of an absolute neutrophil count of at least 1,000 cells/mm³ and a platelet count of at least 75,000 cells/mm³ within 10 days
Any other grade 4+ hematologic toxicity lasting ≥5 days
Grade 3+ ALT or AST in combination with a bilirubin >2 times ULN with no evidence of cholestasis or another cause such as viral infection or other drugs (i.e. Hy's law)
Grade 3 infusion-related reaction that occurs with premedication; Grade 4 infusion-related reaction
Grade 3 Vascular Leak Syndrome defined as hypotension associated with fluid retention and pulmonary edema
Grade 3+ anaphylaxis
Grade 3+ hypotension
Grade 3+AE that does not resolve to grade <2 within 7 days of starting accepted standard of care medical management
Grade 3+ cytokine release syndrome

The following exceptions applied to non-hematologic AEs:
Grade 3 fatigue, nausea, vomiting, or diarrhea that resolves to grade ≤2 with optimal medical management in ≤3 days
Grade 3 fever (as defined by >40° C. for ≤24 hours)
Grade 3 infusion-related reaction that occurs without premedication; subsequent doses should use premedication and if reaction recurs then it will be a DLT
Grade 3 arthralgia or rash that resolves to grade ≤2 within 7 days of starting accepted standard of care medical management (e.g. systemic corticosteroid therapy)
   If a subject had grade 1 or 2 ALT or AST elevation at baseline considered secondhand to liver metastases, a grade 3 elevation must also be ≥3 times baseline and last >7 days.

Serious AEs were defined as any AE that results in any of the following outcomes: Death; Life-threatening AE; Inpatient hospitalization or prolongation of an existing hospitalization; A persistent or significant incapacity or substantial disruption of the ability to conduct normal life functions; or a congenital anomaly/birth defect. Important medical events that may not result in death, be life-threatening, or require hospitalization may be considered serious when, based upon appropriate medical judgment, they may jeopardize the subject and may require medical or surgical intervention to prevent one of the outcomes listed above. Examples of such medical events include allergic bronchospasm requiring intensive treatment in an emergency room or at home, blood dyscrasias or convulsions that do not result in inpatient hospitalization, or the development of drug dependency or drug abuse.

There were no dose-limiting toxicities reported at either dose and there were no treatment-related adverse events (TRAE) leading to discontinuation. Two TRAEs led to dosage reduction. There were 5 treatment-related serious AEs reported in three of the six patients treated at 16 μg/kg dose IV Q3W.

At least 9 subjects experienced TRAEs. The most common TRAEs (>2 patients) of all grades by SOC included general disorders and administration conditions (9/10), investigations (6/10 subjects), metabolism and nutrition (4/10), nervous system disorders (4/10), respiratory, thoracic and mediastinal disorders (4/10), vascular disorders (3/10), skin and subcutaneous disorders (3/10), blood and lymphatic disorders, cardiac disorders, gastrointestinal disorders, immune system disorders, infections and infestations, and musculoskeletal (2/10). TEAEs by preferred terms are detailed in Table 4.

TABLE 4

| Adverse Events (PT), n (%) | Frequency (N = 10) |
|---|---|
| Anemia | 2 (20%) |
| Influenza-Like Illness | 4 (40%) |
| Pyrexia | 6 (60%) |
| Chills | 4 (40%) |
| Fatigue | 5 (50%) |
| Nausea | 2 (20%) |
| Vomiting | 2 (20%) |
| ALT increase | 4 (40%) |
| AST Increase | 4 (40%) |
| Decreased Appetite | 1 (10%) |
| Hypophosphatemia | 3 (30%) |
| Lymphocyte Count Decreased | 2 (20%) |
| Hypotension | 3 (30%) |

Treatment-related AEs were transient and resolved with accepted standard of care. AEs of fever, hypotension, and hypoxia did not correlate with IL-5/IL-6 cytokine elevation. No cumulative toxicity, end organ toxicity, vascular leak syndrome, or eosinophilia was observed. IL-5 levels remained at or below the lowest level of detection. One subject had G2 hypotension which resolved with hydration. One subject had G3 cytokine release syndrome (fever+hypotension requiring pressors; subject had baseline orthostatic hypotension) resulting in dose reduction. There was no notable impact to vital signs, no QTc prolongation, or other cardiac toxicity. Accordingly, the IL-2 conjugate in combination with pembrolizumab demonstrated encouraging PD data and was generally well-tolerated with no discontinuations due to TRAE. It was determined that the in vivo half-life of the IL-2 conjugate was about 10 hours. Overall, the results are considered to support non-alpha preferential activity of the IL-2 conjugate, with a tolerable safety profile in combination with pembrolizumab as well as encouraging PD and preliminary evidence of activity in patients with immune-sensitive tumors.

Cohort Treated with 24 μg/Kg Dose

Six individuals (male [100%], 4 [66.7%] caucasian) with a median age of 51.5 years, ranging from 46-66 years of age, having advanced or metastatic solid tumors received the IL-2 conjugate at a 24 μg/kg dose Q3W. Tumor types included lung cancer, basal cell carcinoma, and colon cancer.

Each subject was treated with a) the IL-2 conjugate administered via IV infusion at a dose of 24 μg/kg for 30 minutes, and b) pembrolizumab administered at a dose of 200 mg IV sequentially. Treatment was given every 3 weeks [Q3W]. Effects on the same biomarkers described above for the 8 μg/kg and 16 μg/kg doses of the IL-2 conjugate were analyzed as surrogate predictors of safety and/or efficacy. Subjects in these studies met the same criteria as the subjects treated 8 μg/kg and 16 μg/kg doses.

One 67-year-old male with head and neck squamous cell carcinoma receiving 32 μg/kg IL-2 conjugate and pembrolizumab achieved a partial response at Cycle 3 (47% decrease in tumor volume). The subject had a history of HNSCC of larynx and had previously received 4 lines of systemic therapy including: carbo Taxol (for a duration of about three months) and then for about another three months, pembrolizumab (for a duration of about three months) and recently CX-2029 a probody drug targeting CD71. The subject had also received radiotherapy. The subject was hospitalized prior to Cycle 5 with hypoxia and found to have bilateral pleural effusions presumed to be disease progression and passed away.

Five (83.3%) of 6 subjects experienced at least one TEAE, and 4 (66.7%) of 6 subjects experienced at least 1 Grade 3-4 related TEAEs (1 Grade 3 and 3 Grade 4). There was one Grade 3 ALT/AST elevation (also with Grade 3 hypophosphatemia) and 3 Grade 4 lymphocyte count decrease (one in a subject with Grade 3 AST/ALT elevation, Grade 2 hyperbilirubinemia-DLT along with Grade 2 CRS). The lymphocyte count recovered to at least Grade 3 in 48 hours.

Two subjects experienced related SAEs: one Grade 1 fever in a subject with adrenal insufficiency requiring steroid adjustment, and one Grade 2 cytokine release syndrome (fever and hypotension requiring fluids and dexamethasone) associated with Grade 3 AST/ALT elevation and G2 hyperbilirubinemia. There was one instance of a DLT: a subject with Grade 3 AST/ALT elevation along with Grade 2 hyperbilirubinemia associated with Grade 2 CRS (fever and hypotension requiring hydration and dexamethasone). For this subject, the dose was reduced for C2D1. No drug discontinuations resulted from TEAs. TEAs are detailed in Table 5.

TABLE 5
Treatment Emergent Adverse Events (TEAE) (n = 6)

| System Organ Class | Grade 1 | Grade 2 | Grade 3 | Grade 4 | Grade 5 |
|---|---|---|---|---|---|
| Blood and lymphatic disorders | 0/6 (0%) | 1/6 (16.7%) | 1/6 (16.7%) | 0/6 (0%) | 0/6 (0%) |
| Cardiac disorders | 1/6 (16.7%) | 0/6 (0%) | 0/6 (0%) | 0/6 (0%) | 0/6 (0%) |
| General disorders and | 2/6 (33.3%) | 2/6 (33.3%) | 0/6 (0%) | 0/6 (0%) | 0/6 (0%) |
| administration site conitions | | | | | |
| Gastrointestinal disorders | 3/6 (50%) | 0/6 (0%) | 0/6 (0%) | 0/6 (0%) | 0/6 (0%) |
| Hepatobiliary Disorders | 0/6 (0%) | 1/6 (16.7%) | 0/6 (0%) | 0/6 (0%) | 0/6 (0%) |
| Immune system disorders | 0/6 (0%) | 1/6 (16.7%) | 0/6 (0%) | 0/6 (0%) | 0/6 (0%) |
| Investigations | 0/6 (0%) | 0/6 (0%) | 1/6 (16.7%) | 3/6 (50%) | 0/6 (0%) |
| Metabolism and nutrition | 1/6 (16.7%) | 2/6 (33.3%) | 1/6 (16.7%) | 0/6 (0%) | 0/6 (0%) |
| disorders | | | | | |
| Musculoskeletal and | 1/6 (16.7%) | 2/6 (33.3%) | 0/6 (0%) | 0/6 (0%) | 0/6 (0%) |
| connective tissue disorders | | | | | |
| Psychiatric disorders | 0/6 (0%) | 1/6 (16.7%) | 0/6 (0%) | 0/6 (0%) | 0/6 (0%) |
| Respiratory, thoracic and | 1/6 (16.7%) | 0/6 (0%) | 1/6 (16.7%) | 0/6 (0%) | 0/6 (0%) |
| mediastinal disorders | | | | | |
| Skin and subcutaneous | 1/6 (16.7%) | 0/6 (0%) | 0/6 (0%) | 0/6 (0%) | 0/6 (0%) |
| disorders | | | | | |
| Vascular disorders | 1/6 (16.7%) | 0/6 (0%) | 0/6 (0%) | 0/6 (0%) | 0/6 (0%) |

The following related events were reported: one Grade 3 AST/ALT and Grade 2 bilirubin (DLT) in the setting of Grade 2 CRS (fever, hypotension [BP97/56 mm Hg] and hypoxemia [SpO2 92%]) managed with fluid bolus, supplemental oxygen and dexamethasone with resolution required a dose reduction for C2D1; one patient fever, chills, rigors and hypoxemia (92%) requiring supportive care and oxygen (C2D1); one Grade 3 AST/ALT (C2D8) presumed related to IL-2 conjugate and pembrolizumab without other symptoms in the setting of alcoholism; and three Grade 4 lymphocyte count decrease.

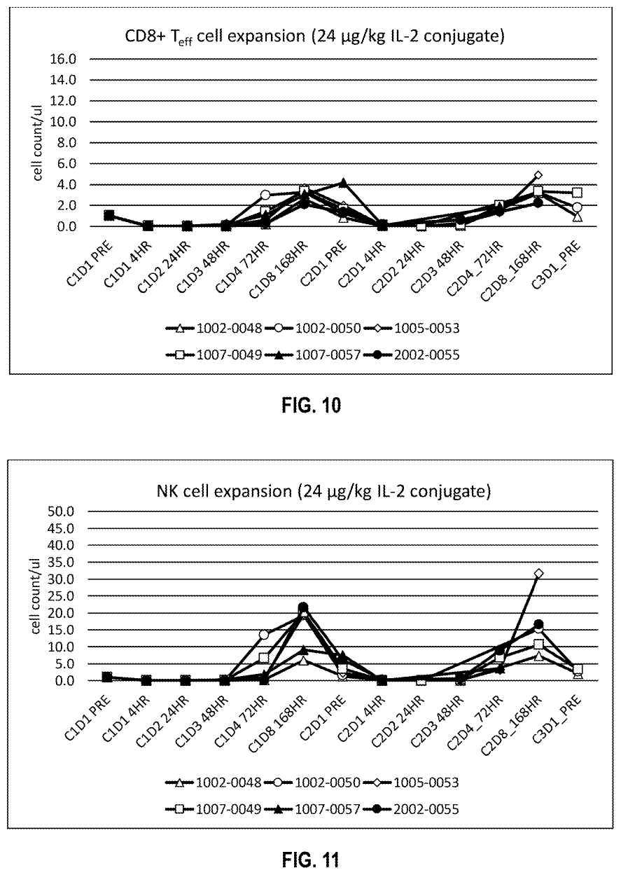

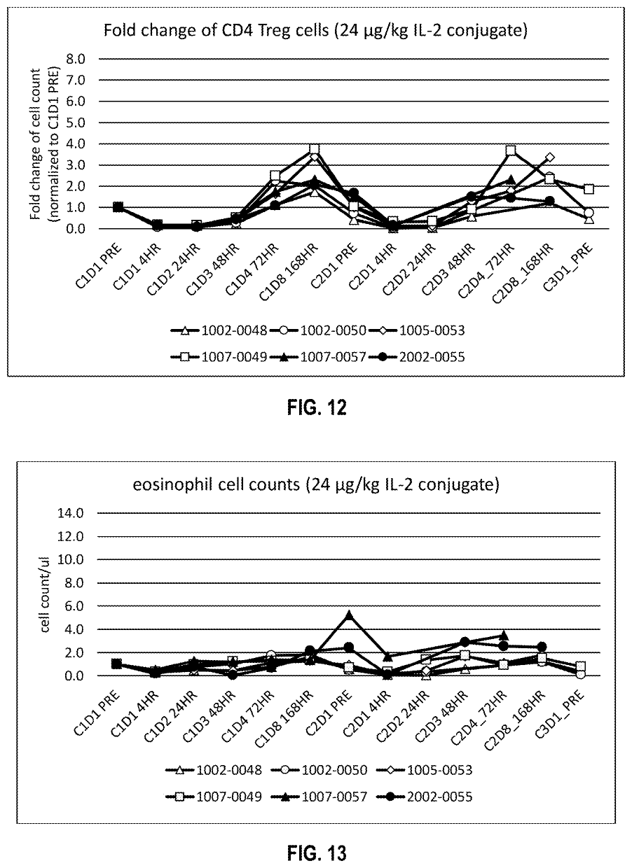

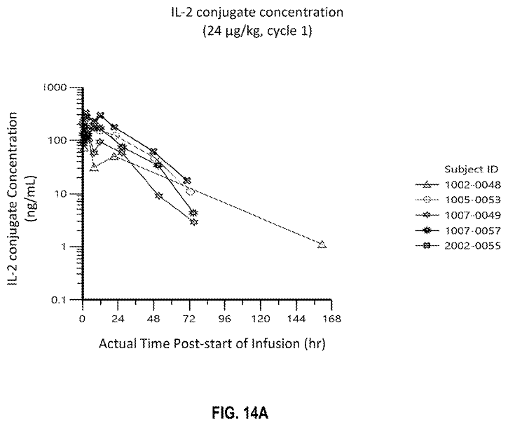

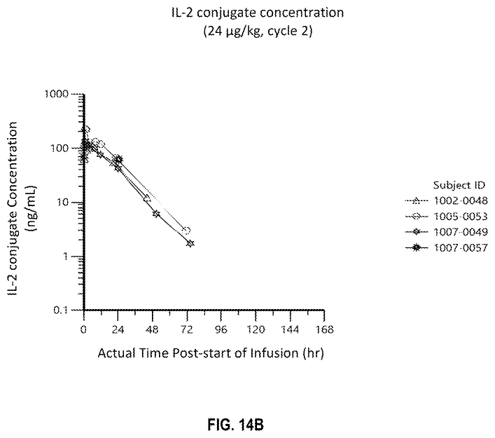

Efficacy biomarkers. Peripheral CD8+T_{eff} cell counts were measured (FIG. 10), and peripheral NK cell counts are shown in FIG. 11. Peripheral CD4+T_{reg} cell counts are shown in FIG. 12, and peripheral eosinophil cell counts are shown in FIG. 13.

Mean concentrations of the IL-2 conjugate after 1 and 2 cycles are shown in FIG. 14A and FIG. 14B, respectively.

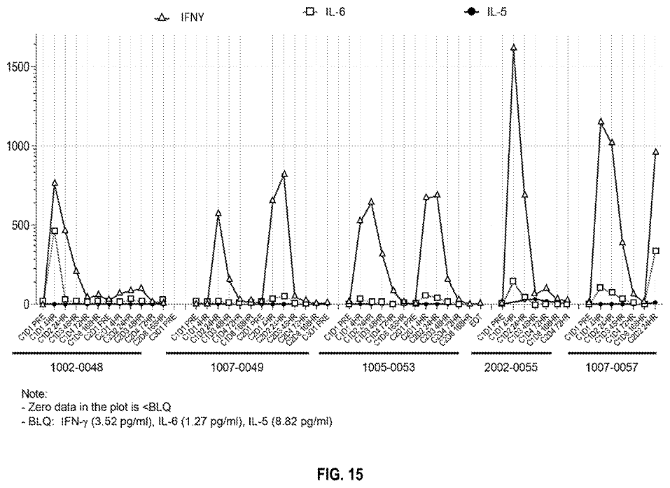

Cytokine levels (IFN-γ, IL-6, and IL-5) are shown in FIG. 15.

Accordingly, the IL-2 conjugate in combination with pembrolizumab demonstrated encouraging PD data and was generally well-tolerated with no discontinuations due to TRAE. Overall, the results are considered to support non-alpha preferential activity of the IL-2 conjugate, with a tolerable safety profile in combination with pembrolizumab as well as encouraging PD and preliminary evidence of activity in patients with immune-sensitive tumors.

Cohort Treated with 32 μg/Kg Dose

Three individuals having advanced or metastatic solid tumors received the IL-2 conjugate at a 32 μg/kg dose Q3W. Tumor types included ovarian carcinoma.

Each subject was treated with a) the IL-2 conjugate administered via IV infusion at a dose of 32 μg/kg for 30 minutes, and b) pembrolizumab administered at a dose of 200 mg IV sequentially. Treatment was given every 3 weeks [Q3W]. Effects on the same biomarkers described above for the 8 μg/kg and 16 μg/kg IL-2 conjugate doses were analyzed as surrogate predictors of safety and/or efficacy. Subjects in these studies met the same criteria as the subjects treated 8 μg/kg and 16 μg/kg doses.

All three (100%) subjects experienced at least one TEAE, and one (33.3%) of 3 subjects experienced at least 1 Grade 3-4 related TEAEs (1 Grade 4). There was one instance of Grade 4 lymphocyte count decrease (subject also had G3 fever). There was one related SAEs of Grade 1 fever and Grade 1 tachycardia requiring hospitalization for 24 hours (C2D2-C2D3). This was resolved with supportive care. There were no DLTs and no drug discontinuations resulting from TEAEs. TEAEs are detailed in Table 6.

TABLE 6
Treatment Emergent Adverse Events (TEAE) (n = 3)

| System Organ Class | Grade 1 | Grade 2 | Grade 3 | Grade 4 | Grade 5 |
|---|---|---|---|---|---|
| Blood and lymphatic disorders | 0/3 (0%) | 1/3 (33.3%) | 0/3 (0%) | 0/3 (0%) | 0/3 (0%) |
| Cardiac disorders | 1/3 (33.3%) | 0/3 (0%) | 0/3 (0%) | 0/3 (0%) | 0/3 (0%) |
| Endocrine disorders | 0/3 (0%) | 1/3 (33.3%) | 0/3 (0%) | 0/3 (0%) | 0/3 (0%) |
| Gastrointestinal disorders | 1/3 (33.3%) | 1/3 (33.3%) | 0/3 (0%) | 0/3 (0%) | 0/3 (0%) |
| General disorders and | 2/3 (66.6%) | 1/3 (33.3%) | 0/3 (0%) | 0/3 (0%) | 0/3 (0%) |
| administration conditions | | | | | |
| Immune system disorders | 0/3 (0%) | 1/3 (33.3%) | 0/3 (0%) | 0/3 (0%) | 0/3 (0%) |
| Infections and infestations | 1/3 (33.3%) | 0/3 (0%) | 0/3 (0%) | 0/3 (0%) | 0/3 (0%) |
| Investigations | 0/3 (0%) | 2/3 (66.6%) | 0/3 (0%) | 1/3 (33.3%) | 0/3 (0%) |
| Metabolism and nutrition | 0/3 (0%) | 2/3 (66.6%) | 0/3 (0%) | 0/3 (0%) | 0/3 (0%) |
| disorders | | | | | |
| Nervous system disorders | 1/3 (33.3%) | 0/3 (0%) | 0/3 (0%) | 0/3 (0%) | 0/3 (0%) |
| Respiratory, thoracic and | 1/3 (33.3%) | 0/3 (0%) | 0/3 (0%) | 0/3 (0%) | 0/3 (0%) |
| mediastinal disorders | | | | | |
| Skin and subcutaneous | 1/3 (33.3%) | 0/3 (0%) | 0/3 (0%) | 0/3 (0%) | 0/3 (0%) |
| disorders | | | | | |
| Vascular disorders | 1/3 (33.3%) | 0/3 (0%) | 0/3 (0%) | 0/3 (0%) | 0/3 (0%) |

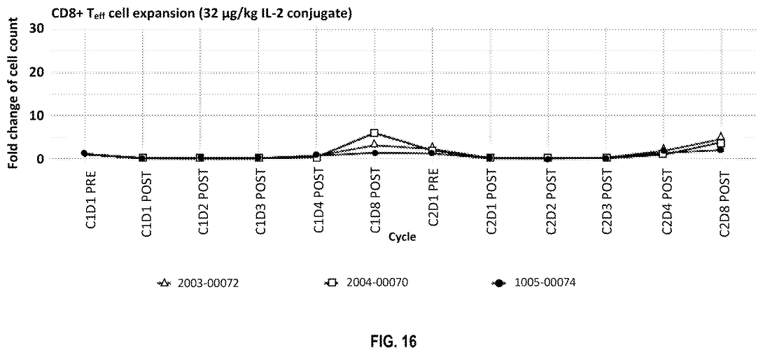

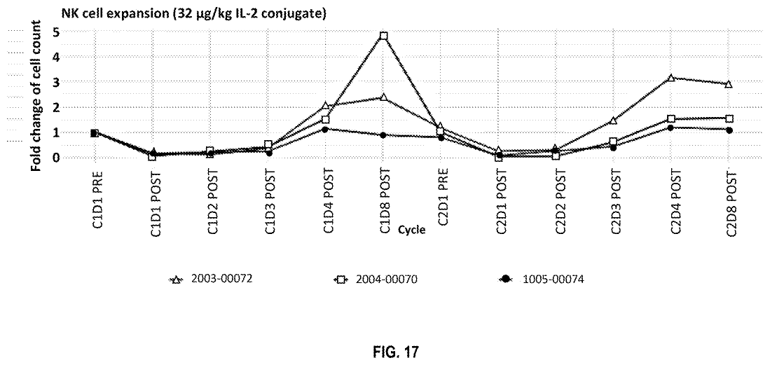

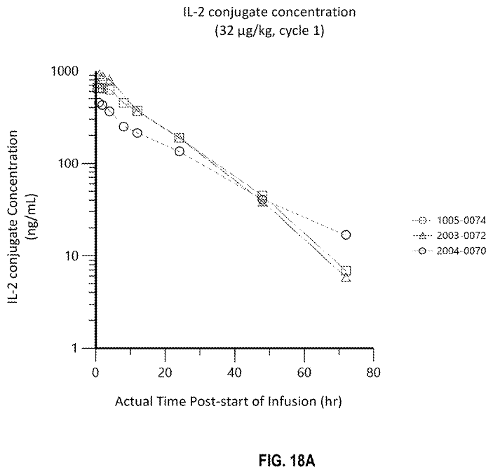

Efficacy biomarkers. Peripheral CD8+T_{eff} cell counts were measured (FIG. 16). Peripheral CD4+T_{reg} cell counts are shown in FIG. 17.

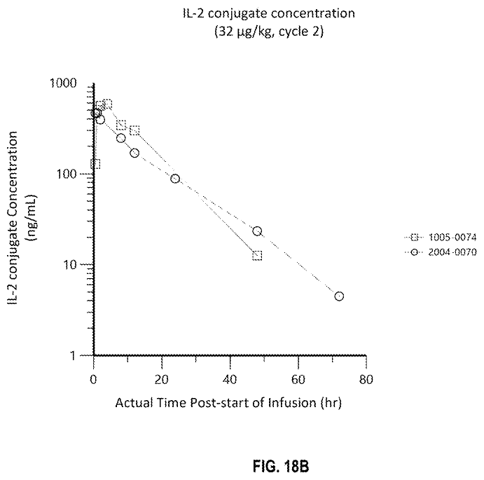

Mean concentrations of the T1L-2 conjugate after 1 and 2 cycles are shown in FIG. 18A and FIG. 18B, respectively.

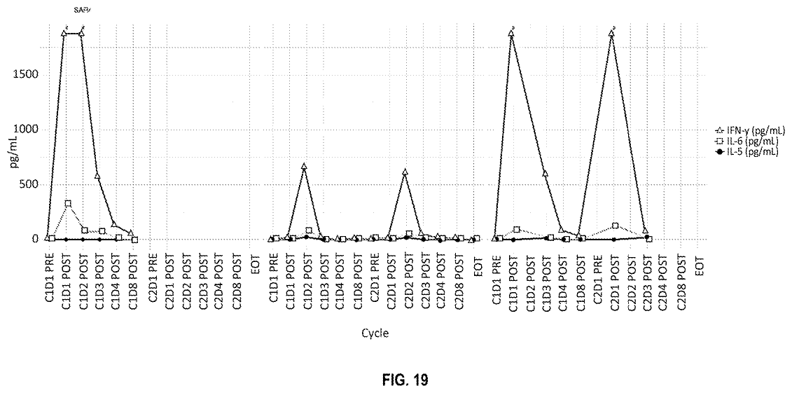

Cytokine levels (IFN-γ, T1L-6, and T1L-5) are shown in FIG. 19.

Accordingly, the T1L-2 conjugate in combination with pembrolizumab demonstrated encouraging PD data and was generally well-tolerated with no discontinuations due to TEAL. Overall, the results are considered to support non-alpha preferential activity of the T1L-2 conjugate, with a tolerable safety profile in combination with pembrolizumab as well as encouraging PD and preliminary evidence of activity in patients with immune-sensitive tumors.

### Example 3. Clinical Study of Combination Therapy Using an IL-2 Conjugate and Pembrolizumab in Subjects Having a PD-L1 Combined Positive Scope (CPS) Greater than or Equal to 1 (Cohort A1)

Preclinical studies have demonstrated that the IL-2 conjugate of Example 2 leads to polyclonal expansion of CD8+ T cells in murine and non-human primate (NHP) models. Studies have also shown that an anti-PD-1 antibody prevents T cell suppression through the PD-1/PD-L1 pathway. In a syngeneic mouse Ct-26 colon cancer model, treatment using an anti-PD-1 antibody in combination with the IL-2 conjugate demonstrated enhanced anti-tumor activity and prolonged survival compared to each monotherapy. These data support evaluation of the IL-2 conjugate in combination with pembrolizumab for treatment of HNSCC.

A Phase 2 non-randomized, open-label, multi-cohort, multi-center study assessing the clinical benefit of the IL-2 conjugate described in Example 2 in combination with pembrolizumab for the treatment of participants with HNSCC was undertaken. Cohort A1 participants were patients with HNSCC who are treatment-naïve (1L) for recurrent/metastatic disease and who have a PD-L1 combined positive score (CPS) greater than or equal to 1. Participants were males or females and are aged ≥18 years. Participants must have had at least one measurable lesion per RECIST v1.1 and a histologically or cytologically confirmed diagnosis of R/M HNSCC that was considered not amenable to further therapy with curative intent (eligible primary tumor locations: oropharynx, oral cavity, hypopharynx, and larynx). Participants with oropharyngeal status must have had known human papillomavirus p16 status. Participants must have had adequate cardiovascular, liver, and renal function and laboratory parameters.

Participants were subject to the following exclusion criteria:
ECOG performance status of 2
History of allogenic tissue/solid organ transplant
Immune-mediated/related toxicity from prior immunooncology therapy of Grade 4 or leading to discontinuation
Ongoing AEs caused by any prior anti-cancer therapy ≥Grade 2
Baseline oxygen saturation (SpO2)≤92% (without oxygen therapy)
Prior IL2-based anticancer treatment
Cannot temporarily (for at least 36 hours) withhold antihypertensive medications prior to each dose of the IL-2 conjugate
Any medical or clinical condition, laboratory abnormality, or any specific situation as judged by the Investigator that would preclude protocol therapy or would make the subject inappropriate for the study
Prior treatment with an agent that blocks the PD-1/PD-L1 pathway

Participants of Cohort A1 received the IL-2 conjugate (16 or 24 μg/kg dose) and pembrolizumab (200 mg) by IV infusion once every 3 weeks. The infusion time of the IL-2 conjugate and pembrolizumab was about 30 minutes each. For the first 4 cycles of treatment, prior to administering the IL-2 conjugate, all participants received IL-2 conjugate premedication to prevent or reduce the acute effect of infusion-associated reactions (IAR) or flu-like symptoms, 30 to 60 minutes prior to infusion of the IL-2 conjugate. The IL-2 conjugate premedication was as follows: acetaminophen (about 650-1000 mg, oral), diphenhydramine (about 25-50 mg, intravenous), and/or ondansetron (about 8 mg or 0.15 mg/kg, intravenous). After the first 4 cycles, administration of the IL-2 conjugate premedication was optional based on the supervising physician's assessment. The dosing sequence was as follows: (i) pembrolizumab; (ii) premedication for the IL-2 conjugate (administered 30-60 min. prior to the start of the IL-2 conjugate infusion); and (iii) IL-2 conjugate (the start of IL-2 conjugate infusion will be at least 30 min. after completion of pembrolizumab infusion). Treatment was repeated for up to a total of 35 cycles or for a duration up to 735 days.

The progression of disease was monitored in patients according to various criteria. The objective response rate (ORR) was evaluated in patients following administration of the IL-2 conjugate and pembrolizumab combination treatment per RECIST 1.1. The incidence of treatment emergent adverse events (TEAEs), dose-limiting toxicities (DLTs), serious adverse events (SAEs), and laboratory abnormalities were evaluated following administration of the IL-2 conjugate and pembrolizumab combination treatment according to the National Cancer Institute Common Terminology Criteria for Adverse Events (NCI CTCAE) v 5.0 and the American Society for Transplantation and Cellular Therapy (ASTCT) consensus gradings. The time to complete response (CR) or partial response (PR) per RECIST 1.1 was evaluated for patients following administration of the IL-2 conjugate and pembrolizumab combination treatment.

The following parameters were also evaluated in patients following administration of the IL-2 conjugate and pembrolizumab combination treatment: (1) duration of response (DoR), defined as the time from the first documented evidence of CR or PR until progressive disease (PD) determined per RECIST 1.1 or death from any cause, whichever occurs first; (2) clinical benefit rate (CBR), including confirmed CR or PR at any time or stable disease (SD) of at least 6 months per RECIST 1.1; and (3) progression free survival (PFS), defined as the time from the date of first administration of IL-2 conjugate and pembrolizumab combination treatment to the date of the first documented tumor progression, as per RECIST 1.1 or death due to any cause, whichever occurs first. Pharmacokinetic parameters, such as concentration of the IL-2 conjugate and incidence of anti-drug antibodies (ADAs) against the IL-2 conjugate, can also be evaluated in patients at various time points throughout the study. The following additional indicators of anti-tumor activity were also evaluated in patients following administration of the IL-2 conjugate and pembrolizumab combination treatment: (1) objective response rate by immune Response Evaluation Criteria in Solid Tumors for immune-based therapies (iRECIST); (2) disease control rate (DCR), defined as the proportion of participants who have achieved CR, PR, or SD, per RECIST 1.1; (3) complete response rate (CRR), defined as the proportion of participants who have a confirmed CR, determined per RECIST 1.1; and (4) OS, defined as the time from the first dose of the IL-2 conjugate and pembrolizumab combination treatment to the date of death due to any cause. The following additional effects on the immune system were also evaluated in patients following administration of the IL-2 conjugate and pembrolizumab combination treatment: (1) immune cells expansion and kinetics in blood (CD8+ T-cells and NK cells proliferation (Ki67) and expansion in blood); (2) modulation of immune response in the tumor microenvironment (TME) (programmed death-ligand 1 (PD-L1), CD8+/Ki67, CD4+, FoxP3, and T/NK/Treg expression on baseline and on-treatment tumor tissue samples); (3) kinetics of cytokine production (cytokine panel in blood); and (4) predictive markers of responses (PD-L1, mismatch repair status, tumor mutation burden (TMB), immune gene signature, circulating tumor DNA (ctDNA) on baseline samples).

Results. Five subjects having R/M HNSCC, PD-L1 CPS≥1 (Cohort A1) received the IL-2 conjugate at a dose of 24 μg/kg Q3W in combination with pembrolizumab (300 mg Q3W). Three of the subjects had at least one evaluable post-baseline tumor assessment scan (i.e., were evaluated for efficacy). For 1 of the 3 subjects, investigators reported an unconfirmed partial response (i.e., an apparent decrease in the size of target lesions). The other 2 subjects had a Best Overall Response (BOR) of stable disease without pending CR/PR confirmation. Evaluations are not yet available for the other subjects.

Treatment-emergent adverse events (TEAEs) are summarized in Table 7.

TABLE 7
Treatment Emergent Adverse Events: n = 5

| System Organ Class | All Grades | Grade ≥ 3 |
|---|---|---|
| Infections and Infestations | 3/5 (60.0%) | 1/5 (20%) |
| Blood and lymphatic system disorders | 2/5 (40.0%) | 0/5 (0%) |
| Immune system disorders | 1/5 (20.0%) | 0/5 (0%) |
| Metabolism and nutrition disorders | 0/5 (0%) | 0/5 (0%) |
| Psychiatric | 0/5 (0%) | 0/5 (0%) |
| Nervous system disorders | 0/5 (0%) | 0/5 (0%) |
| Cardiac disorders | 0/5 (0%) | 0/5 (0%) |
| Vascular disorders | 2/5 (40.0%) | 0/5 (0%) |
| Respiratory, Thoracic, and Mediastinal | 1/5 (20.0%) | 1/5 (20.0%) |
| disorders | | |
| Gastrointestinal disorders | 3/5 (60.0%) | 2/5 (40.0%) |
| Skin and subcutaneous tissue disorders | 1/5 (20.0%) | 0/5 (0%) |
| Musculoskeletal and connective tissue | 0/5 (0%) | 0/5 (0%) |
| disorders | | |
| Renal and urniary | 0/5 (0%) | 0/5 (0%) |
| General disorders and administration site | 2/5 (40.0%) | 1/5 (20.0%) |
| conditions | | |
| Injury, Poisoning, and Procedural | 2/5 (40.0%) | 1/5 (20.0%) |
| Complications | | |
| Acute febrile illness secondary to capillary | 1/5 (20.0%) | 0/5 (0%) |
| leak syndrome | | |

In some embodiments, an individual shows a decrease in the size of target lesion(s) after one cycle of treatment. In some embodiments, an individual shows a decrease in the size of target lesion(s) after the first tumor assessment. In some embodiments, an individual shows a response (i.e., a decrease in the size of target lesions) after the second, third, or fourth tumor assessment. In some embodiments, the individual shows a response (i.e., a decrease in the size of target lesions) after 1, 2, 3, 4, 5, 6, 7, 8, 9, or 10 cycles of treatment. In some embodiments, the individual shows a response (i.e., a decrease in the size of target lesions) after 1, 2, 3, 4, 5, 6, 7, 8, 9, 10, 12, 14, 16, 18, or 20 weeks following the first treatment.

### Example 4. Clinical Study of Combination Therapy Using an IL-2 Conjugate, Pembrolizumab, and Cetuximab in Subjects Having a PD-L1 Combined Positive Scope (CPS) Greater than or Equal to 1 (Cohort A2)

Monotherapy using the IL-2 conjugate of Example 2 has been demonstrated to promote a peripheral increase in the number of NK cells, which are important effector cells mediating antibody-dependent cellular cytotoxicity (ADCC) for IgG1 antibodies such as cetuximab. Furthermore, in vitro data have shown that NK cell-mediated lysis of head and neck tumor cells is significantly enhanced by the combination of cetuximab therapy with immune stimulatory cytokines, including IL-2 (Luedke, E. et al., Surgery, 2012, 152(3):431-40). Cetuximab can prime the immune system for anti-PD-1 therapy by recruiting cytotoxic cell effectors of both the innate and adaptive immune systems to the intratumoral space (Ferris, R. L. et al., Cancer Treat Rev., 2018, 63:48-60). In addition, associated negative feedback loops lead to upregulation of PD-1/PD-L1-mediated immunosuppression of active cytotoxic cell types, an issue that could be overcome successfully via combination therapy with anti-PD-1 therapy. Collectively, the combination of the IL-2 conjugate with pembrolizumab and cetuximab is supported by: a) increase in NK cells by the IL-2 conjugate; b) the ability of cytokines such as IL-2 to improve the ADCC response of cetuximab; and c) addition of an anti-PD-1 antibody to prevent PD-1/PD-L1 inhibition of infiltrating cytotoxic immune cells.

A Phase 2 non-randomized, open-label, multi-cohort, multi-center study assessing the clinical benefit of the IL-2 conjugate described in Example 2 in combination with pembrolizumab and cetuximab for the treatment of participants with HNSCC is undertaken. Cohort A2 participants are patients with HNSCC who are treatment-naïve (1L) for recurrent/metastatic disease and who have a PD-L1 combined positive score (CPS) greater than or equal to 1. Participants are males or females and are aged ≥18 years. Participants must have at least one measurable lesion per RECIST v1.1 and a histologically or cytologically confirmed diagnosis of R/M HNSCC that is considered not amenable to further therapy with curative intent (eligible primary tumor locations: oropharynx, oral cavity, hypopharynx, and larynx). Participants with oropharyngeal cancer must have known human papillomavirus p16 status. Participants must have adequate cardiovascular, liver, and renal function and laboratory parameters.

Participants are subject to the following exclusion criteria:
ECOG performance status of 2
History of allogenic tissue/solid organ transplant
Immune-mediated/related toxicity from prior immunooncology therapy of Grade 4 or leading to discontinuation
Ongoing AEs caused by any prior anti-cancer therapy ≥Grade 2
Baseline oxygen saturation (SpO2)≤92% (without oxygen therapy)
Prior IL2-based anticancer treatment
Cannot temporarily (for at least 36 hours) withhold antihypertensive medications prior to each dose of the IL-2 conjugate
Any medical or clinical condition, laboratory abnormality, or any specific situation as judged by the Investigator that would preclude protocol therapy or would make the subject inappropriate for the study
Prior treatment with an agent that blocks the PD-1/PD-L1 pathway
Prior treatment with cetuximab

Participants of Cohort A2 will receive the IL-2 conjugate (24 μg/kg dose) and pembrolizumab (200 mg) by IV infusion once every 3 weeks. Cetuximab will be given on Cycle 1 Day 1 as an initial loading dose of 400 mg/m² infused over 120 minutes (maximum infusion rate 10 mg/min), followed by 250 mg/m² infused over 60 minutes (maximum infusion rate 10 mg/min) for all subsequent doses starting with the Cycle 1 Day 8 administration, until progressive disease (PD). Cetuximab will be given on days 1, 8, and 15 of each 21 day cycle. The infusion time of the IL-2 conjugate and pembrolizumab will be about 30 minutes each. For the first 4 cycles of treatment, prior to administering the IL-2 conjugate, all participants will receive IL-2 conjugate premedication to prevent or reduce the acute effect of infusion-associated reactions (IAR) or flu-like symptoms, 30 to 60 minutes prior to infusion of the IL-2 conjugate. The IL-2 conjugate premedication is as follows: acetaminophen (about 650-1000 mg, oral), diphenhydramine (about 25-50 mg, intravenous), and/or ondansetron (about 8 mg or 0.15 mg/kg, intravenous). After the first 4 cycles, administration of the IL-2 conjugate premedication may be optional based on the supervising physician's assessment. Prior to administration of the first dose of cetuximab, all participants will be pre-medicated with diphenhydramine (about 25 to 50 mg, intravenous). Premedication for subsequent doses of cetuximab may be optional based on the supervising physician's assessment. When the IL-2 conjugate and cetuximab are given on the same day, participants who receive diphenhydramine as cetuximab premedication may skip the diphenhydramine as the IL-2 conjugate premedication. The dosing sequence is as follows: (i) pembrolizumab; (ii) premedication for cetuximab (30-60 min. prior to the start of cetuximab infusion); (iii) cetuximab; (iv) premedication for the IL-2 conjugate (administered 30-60 min. prior to the start of the IL-2 conjugate infusion); and (v) IL-2 conjugate. Treatment will be repeated until PD.

The progression of disease can be monitored in patients according to various criteria. The objective response rate (ORR) can be evaluated in patients following administration of the IL-2 conjugate, pembrolizumab, and cetuximab combination treatment per RECIST 1.1. The incidence of treatment emergent adverse events (TEAEs), dose-limiting toxicities (DLTs), serious adverse events (SAEs), and laboratory abnormalities can be evaluated following administration of the IL-2 conjugate, pembrolizumab, and cetuximab combination treatment according to the National Cancer Institute Common Terminology Criteria for Adverse Events (NCI CTCAE) v 5.0 and the American Society for Transplantation and Cellular Therapy (ASTCT) consensus gradings. The time to complete response (CR) or partial response (PR) per RECIST 1.1 can be evaluated for patients following administration of the IL-2 conjugate, pembrolizumab, and cetuximab combination treatment.

The following parameters can also be evaluated in patients following administration of the IL-2 conjugate, pembrolizumab, and cetuximab combination treatment: (1) duration of response (DoR), defined as the time from the first documented evidence of CR or PR until progressive disease (PD) determined per RECIST 1.1 or death from any cause, whichever occurs first; (2) clinical benefit rate (CBR), including confirmed CR or PR at any time or stable disease (SD) of at least 6 months per RECIST 1.1; and (3) progression free survival (PFS), defined as the time from the date of first administration of IL-2 conjugate, pembrolizumab, and cetuximab combination treatment to the date of the first documented tumor progression, as per RECIST 1.1 or death due to any cause, whichever occurs first. Pharmacokinetic parameters, such as concentrations of the IL-2 conjugate and cetuximab, and incidence of anti-drug antibodies (ADAs) against the IL-2 conjugate, can also be evaluated in patients at various time points throughout the study. The following additional indicators of anti-tumor activity can also be evaluated in patients following administration of the IL-2 conjugate, pembrolizumab, and cetuximab combination treatment: (1) objective response rate by immune Response Evaluation Criteria in Solid Tumors for immune-based therapies (iRECIST); (2) disease control rate (DCR), defined as the proportion of participants who have achieved CR, PR, or SD, per RECIST 1.1; (3) complete response rate (CRR), defined as the proportion of participants who have a confirmed CR, determined per RECIST 1.1; and (4) OS, defined as the time from the first dose of the IL-2 conjugate, pembrolizumab, and cetuximab combination treatment to the date of death due to any cause. The following additional effects on the immune system can also be evaluated in patients following administration of the IL-2 conjugate, pembrolizumab, and cetuximab combination treatment: (1) immune cells expansion and kinetics in blood (CD8+ T-cells and NK cells proliferation (Ki67) and expansion in blood); (2) modulation of immune response in the tumor microenvironment (TME) (programmed death-ligand 1 (PD-L1), CD8+/Ki67, CD4+, FoxP3, and T/NK/Treg expression on baseline and on-treatment tumor tissue samples); (3) kinetics of cytokine production (cytokine panel in blood); and (4) predictive markers of responses (PD-L1, mismatch repair status, tumor mutation burden (TMB), immune gene signature, circulating tumor DNA (ctDNA) on baseline samples).

In some embodiments, an individual shows a decrease in the size of target lesion(s) after one cycle of treatment. In some embodiments, an individual shows a decrease in the size of target lesion(s) after the first tumor assessment. In some embodiments, an individual shows a response (i.e., a decrease in the size of target lesions) after the second, third, or fourth tumor assessment. In some embodiments, the individual shows a response (i.e., a decrease in the size of target lesions) after 1, 2, 3, 4, 5, 6, 7, 8, 9, or 10 cycles of treatment. In some embodiments, the individual shows a response (i.e., a decrease in the size of target lesions) after 1, 2, 3, 4, 5, 6, 7, 8, 9, 10, 12, 14, 16, 18, or 20 weeks following the first treatment.

### Example 5. Clinical Study of Combination Therapy Using an IL-2 Conjugate, Pembrolizumab, and an Anti-TGFβ Antibody in Subjects Having a PD-L1 Combined Positive Scope (CPS) Greater than or Equal to 1 (Cohort A3)

In this study, the IL-2 conjugate and pembrolizumab are intended to expand innate and adaptive immune cells while relieving PD-1/PD-L1 mediated immune suppression, respectively, whereas TGFβ exerts strong immune suppressive effects within the tumor microenvironment which blunts all aspects of the anti-tumor immune response (Akhurst, R. J. et al., Nat. Rev. Drug Discov., 2012, 11(10):790-811). Addition of an anti-TGFβ antibody is expected to overcome prominent immune evasion mechanisms and boost anti-cancer T cell immunity that may bring meaningful clinical benefit. Preclinical murine syngeneic tumor models have demonstrated the combination of IL-2 and an anti-TGFβ antibody significantly increased the survival of metastatic tumor-bearing mice compared with IL-2 alone (Alverez, M. et al., J. Immunol., 2014, 193(4):1709-16), and other studies have shown that co-administration of TGFβ-blocking and anti-PD-L1 antibodies provoked vigorous anti-tumor immunity and tumor regression (Mariathasan, S. et al., Nature, 2018, 554(7693):544-48). For patients resistant to anti-PD-1 antibody monotherapy, the combination of TGFβ inhibition via an anti-TGFβ antibody with anti-PD-1 antibody may provide benefit due to the inhibition of the mechanism responsible for the resistance, namely TGFβ.

A Phase 2 non-randomized, open-label, multi-cohort, multi-center study assessing the clinical benefit of the IL-2 conjugate described in Example 2 in combination with pembrolizumab and an anti-TGFβ antibody (SAR439459; Greco, R. et al., OncoImmunology, 2020, 9:1, e1811605) for the treatment of participants with HNSCC is undertaken. The anti-TGFβ antibody used in this example is a human anti-TGFβ monoclonal antibody that neutralizes all isoforms of TGFβ and has a high sequence similarity to fresolimumab (GC1008), differing only by a single amino acid in the heavy chain (S228P, according to the EU numbering scheme). Cohort A3 participants are patients with HNSCC who are treatment-naïve for recurrent/metastatic disease and who have a PD-L1 combined positive score (CPS) greater than or equal to 1.

Participants of Cohort A3 will receive the IL-2 conjugate (16 or 24 μg/kg dose), pembrolizumab (200 mg), and the anti-TGFβ antibody (22.5 mg/kg) by IV infusion once every 3 weeks. The infusion time of the IL-2 conjugate, pembrolizumab, and anti-TGFβ antibody will be about 30 minutes each. For the first 4 cycles of treatment, prior to administering the IL-2 conjugate, all participants will receive IL-2 conjugate premedication to prevent or reduce the acute effect of infusion-associated reactions (IAR) or flu-like symptoms, 30 to 60 minutes prior to infusion of the IL-2 conjugate. The IL-2 conjugate premedication is as follows: acetaminophen (about 650-1000 mg, oral), diphenhydramine (about 25-50 mg, intravenous), and/or ondansetron (about 8 mg or 0.15 mg/kg, intravenous). After the first 4 cycles, administration of the IL-2 conjugate premedication may be optional based on the supervising physician's assessment. The dosing sequence is as follows: (i) pembrolizumab; (ii) premedication for the IL-2 conjugate (administered 30-60 min. prior to the start of the IL-2 conjugate infusion, may be given before pembrolizumab); (iii) IL-2 conjugate (the start of IL-2 conjugate infusion will be at least 30 min. after completion of pembrolizumab infusion); and (iv) anti-TGFβ antibody. Treatment will be repeated for up to a total of 35 cycles or for a duration up to 735 days.

The progression of disease can be monitored in patients according to various criteria. The objective response rate (ORR) can be evaluated in patients following administration of the IL-2 conjugate, pembrolizumab, and anti-TGFβ antibody combination treatment per RECIST 1.1. The incidence of treatment emergent adverse events (TEAEs), dose-limiting toxicities (DLTs), serious adverse events (SAEs), and laboratory abnormalities can be evaluated following administration of the IL-2 conjugate, pembrolizumab, and anti-TGFβ antibody combination treatment according to the National Cancer Institute Common Terminology Criteria for Adverse Events (NCI CTCAE) v 5.0 and the American Society for Transplantation and Cellular Therapy (ASTCT) consensus gradings. The time to complete response (CR) or partial response (PR) per RECIST 1.1 can be evaluated for patients following administration of the IL-2 conjugate, pembrolizumab, and anti-TGFβ antibody combination treatment.

The following parameters can also be evaluated in patients following administration of the IL-2 conjugate, pembrolizumab, and anti-TGFβ antibody combination treatment: (1) duration of response (DoR), defined as the time from the first documented evidence of CR or PR until progressive disease (PD) determined per RECIST 1.1 or death from any cause, whichever occurs first; (2) clinical benefit rate (CBR), including confirmed CR or PR at any time or stable disease (SD) of at least 6 months per RECIST 1.1; and (3) progression free survival (PFS), defined as the time from the date of first administration of IL-2 conjugate, pembrolizumab, and anti-TGFβ antibody combination treatment to the date of the first documented tumor progression, as per RECIST 1.1 or death due to any cause, whichever occurs first. Pharmacokinetic parameters, such as concentrations of the IL-2 conjugate and anti-TGFβ antibody, and incidence of anti-drug antibodies (ADAs) against the IL-2 conjugate and anti-TGFβ antibody, can also be evaluated in patients at various time points throughout the study. The following additional indicators of anti-tumor activity can also be evaluated in patients following administration of the IL-2 conjugate, pembrolizumab, and anti-TGFβ antibody combination treatment: (1) objective response rate by immune Response Evaluation Criteria in Solid Tumors for immune-based therapies (iRECIST); (2) disease control rate (DCR), defined as the proportion of participants who have achieved CR, PR, or SD, per RECIST 1.1; (3) complete response rate (CRR), defined as the proportion of participants who have a confirmed CR, determined per RECIST 1.1; and (4) OS, defined as the time from the first dose of the IL-2 conjugate, pembrolizumab, and anti-TGFβ antibody combination treatment to the date of death due to any cause. The following additional effects on the immune system can also be evaluated in patients following administration of the IL-2 conjugate, pembrolizumab, and anti-TGFβ antibody combination treatment: (1) immune cells expansion and kinetics in blood (CD8+ T-cells and NK cells proliferation (Ki67) and expansion in blood); (2) modulation of immune response in the tumor microenvironment (TME) (programmed death-ligand 1 (PD-L1), CD8+/Ki67, CD4+, FoxP3, and T/NK/Treg expression on baseline and on-treatment tumor tissue samples); (3) kinetics of cytokine production (cytokine panel in blood); and (4) predictive markers of responses (PD-L1, mismatch repair status, tumor mutation burden (TMB), immune gene signature, circulating tumor DNA (ctDNA) on baseline samples).

In some embodiments, an individual shows a decrease in the size of target lesion(s) after one cycle of treatment. In some embodiments, an individual shows a decrease in the size of target lesion(s) after the first tumor assessment. In some embodiments, an individual shows a response (i.e., a decrease in the size of target lesions) after the second, third, or fourth tumor assessment. In some embodiments, the individual shows a response (i.e., a decrease in the size of target lesions) after 1, 2, 3, 4, 5, 6, 7, 8, 9, or 10 cycles of treatment. In some embodiments, the individual shows a response (i.e., a decrease in the size of target lesions) after 1, 2, 3, 4, 5, 6, 7, 8, 9, 10, 12, 14, 16, 18, or 20 weeks following the first treatment.

### Example 6. Clinical Study of Combination Therapy Using an IL-2 Conjugate and Pembrolizumab in Subjects Having Platinum-Refractory HNSCC (Cohort B1)

A Phase 2 non-randomized, open-label, multi-cohort, multi-center study assessing the clinical benefit of the IL-2 conjugate described in Example 2 in combination with pembrolizumab for the treatment of participants with HNSCC was undertaken. Cohort B1 participants were patients with platinum-refractory 2L/3L recurrent and/or metastatic HNSCC and who had received prior treatment using a PD-1/PD-L1 based regimen. All Cohort B1 participants have had failure of a checkpoint-based regimen and a platinum-based regimen, and overall failure of no more than 2 regimens for R/M disease. Participants were males or females and are aged ≥18 years. Participants must have had at least one measurable lesion per RECIST v1.1 and a histologically or cytologically confirmed diagnosis of R/M HNSCC that is considered not amenable to further therapy with curative intent (eligible primary tumor locations: oropharynx, oral cavity, hypopharynx, and larynx). Participants with oropharyngeal cancer must have had known human papillomavirus p16 status. Participants must have had adequate cardiovascular, liver, and renal function and laboratory parameters.

Participants were subject to the following exclusion criteria:
ECOG performance status of 2
History of allogenic tissue/solid organ transplant
Immune-mediated/related toxicity from prior immunooncology therapy of Grade 4 or leading to discontinuation
Ongoing AEs caused by any prior anti-cancer therapy ≥Grade 2
Baseline oxygen saturation (SpO2)≤92% (without oxygen therapy)
Prior IL2-based anticancer treatment
Cannot temporarily (for at least 36 hours) withhold antihypertensive medications prior to each dose of the IL-2 conjugate
Any medical or clinical condition, laboratory abnormality, or any specific situation as judged by the Investigator that would preclude protocol therapy or would make the subject inappropriate for the study

Participants of Cohort B1 received the IL-2 conjugate (24 μg/kg dose) and pembrolizumab (200 mg) by IV infusion once every 3 weeks. The infusion time of the IL-2 conjugate and pembrolizumab was about 30 minutes each. For the first 4 cycles of treatment, prior to administering the IL-2 conjugate, all participants received IL-2 conjugate premedication to prevent or reduce the acute effect of infusion-associated reactions (IAR) or flu-like symptoms, 30 to 60 minutes prior to infusion of the IL-2 conjugate. The IL-2 conjugate premedication is as follows: acetaminophen (about 650-1000 mg, oral), diphenhydramine (about 25-50 mg, intravenous), and/or ondansetron (about 8 mg or 0.15 mg/kg, intravenous). After the first 4 cycles, administration of the IL-2 conjugate premedication was optional based on the supervising physician's assessment. The dosing sequence was as follows: (i) pembrolizumab; (ii) premedication for the IL-2 conjugate (administered 30-60 min. prior to the start of the IL-2 conjugate infusion); and (iii) IL-2 conjugate (the start of IL-2 conjugate infusion was at least 30 min. after completion of pembrolizumab infusion). Treatment was repeated for up to a total of 35 cycles or for a duration up to 735 days.

The progression of disease was monitored in patients according to various criteria. The objective response rate (ORR) was evaluated in patients following administration of the IL-2 conjugate and pembrolizumab combination treatment per RECIST 1.1. The incidence of treatment emergent adverse events (TEAEs), dose-limiting toxicities (DLTs), serious adverse events (SAEs), and laboratory abnormalities was evaluated following administration of the IL-2 conjugate and pembrolizumab combination treatment according to the National Cancer Institute Common Terminology Criteria for Adverse Events (NCI CTCAE) v 5.0 and the American Society for Transplantation and Cellular Therapy (ASTCT) consensus gradings. The time to complete response (CR) or partial response (PR) per RECIST 1.1 was evaluated for patients following administration of the IL-2 conjugate and pembrolizumab combination treatment.

The following parameters were also evaluated in patients following administration of the IL-2 conjugate and pembrolizumab combination treatment: (1) duration of response (DoR), defined as the time from the first documented evidence of CR or PR until progressive disease (PD) determined per RECIST 1.1 or death from any cause, whichever occurs first; (2) clinical benefit rate (CBR), including confirmed CR or PR at any time or stable disease (SD) of at least 6 months per RECIST 1.1; and (3) progression free survival (PFS), defined as the time from the date of first administration of IL-2 conjugate and pembrolizumab combination treatment to the date of the first documented tumor progression, as per RECIST 1.1 or death due to any cause, whichever occurs first. Pharmacokinetic parameters, such as concentration of the IL-2 conjugate and incidence of anti-drug antibodies (ADAs) against the IL-2 conjugate, were also evaluated in patients at various time points throughout the study. The following additional indicators of anti-tumor activity were also evaluated in patients following administration of the IL-2 conjugate and pembrolizumab combination treatment: (1) objective response rate by immune Response Evaluation Criteria in Solid Tumors for immune-based therapies (iRECIST); (2) disease control rate (DCR), defined as the proportion of participants who have achieved CR, PR, or SD, per RECIST 1.1; (3) complete response rate (CRR), defined as the proportion of participants who have a confirmed CR, determined per RECIST 1.1; and (4) OS, defined as the time from the first dose of the IL-2 conjugate and pembrolizumab combination treatment to the date of death due to any cause. The following additional effects on the immune system were also evaluated in patients following administration of the IL-2 conjugate and pembrolizumab combination treatment: (1) immune cells expansion and kinetics in blood (CD8+ T-cells and NK cells proliferation (Ki67) and expansion in blood); (2) modulation of immune response in the tumor microenvironment (TME) (programmed death-ligand 1 (PD-L1), CD8+/Ki67, CD4+, FoxP3, and T/NK/Treg expression on baseline and on-treatment tumor tissue samples); (3) kinetics of cytokine production (cytokine panel in blood); and (4) predictive markers of responses (PD-L1, mismatch repair status, tumor mutation burden (TMB), immune gene signature, circulating tumor DNA (ctDNA) on baseline samples).

### Results

Disease status was evaluated for three subjects, each of whom had progressive disease. Treatment-emergent adverse events (TEAEs) are summarized in Table 8.

TABLE 8
Treatment Emergent Adverse Events: n = 5

| System Organ Class | All Grades | Grade ≥ 3 |
|---|---|---|
| Infections and Infestations | 2/5 (40.0%) | 1/5 (20%) |
| Blood and lymphatic system disorders | 0/5 (0%) | 0/5 (0%) |
| Immune system disorders | 1/5 (20.0%) | 0/5 (0%) |
| Metabolism and nutrition disorders | 1/5 (20.0%) | 0/5 (0%) |
| Psychiatric | 0/5 (0%) | 0/5 (0%) |
| Nervous system disorders | 1/5 (20.0%) | 0/5 (0%) |
| Cardiac disorders | 0/5 (0%) | 0/5 (0%) |
| Vascular disorders | 1/5 (20.0%) | 0/5 (0%) |
| Respiratory, Thoracic, and Mediastinal | 1/5 (20.0%) | 0/5 (0%) |
| disorders | | |
| Gastrointestinal disorders | 2/5 (40.0%) | 0/5 (0%) |
| Skin and subcutaneous tissue disorders | 1/5 (20.0%) | 0/5 (0%) |
| Musculoskeletal and connective tissue | 2/5 (40.0%) | 0/5 (0%) |
| disorders | | |
| Renal and urinary | 1/5 (20.0%) | 0/5 (0%) |
| General disorders and Administration site | 5/5 (100%) | 2/5 (40.0%) |
| conditions | | |
| Investigations | 1/5 (20.0%) | 0/5 (0%) |
| Injury, Poisoning, and Procedural | 0/5 (0%) | 0/5 (0%) |
| Complications | | |

In some embodiments, an individual shows a decrease in the size of target lesion(s) after one cycle of treatment. In some embodiments, an individual shows a decrease in the size of target lesion(s) after the first tumor assessment. In some embodiments, an individual shows a response (i.e., a decrease in the size of target lesions) after the second, third, or fourth tumor assessment. In some embodiments, the individual shows a response (i.e., a decrease in the size of target lesions) after 1, 2, 3, 4, 5, 6, 7, 8, 9, or 10 cycles of treatment. In some embodiments, the individual shows a response (i.e., a decrease in the size of target lesions) after 1, 2, 3, 4, 5, 6, 7, 8, 9, 10, 12, 14, 16, 18, or 20 weeks following the first treatment.

### Example 7. In Vitro Study of IL-2 Conjugate and Cetuximab (PBMC ADCC Assay)

A study was performed to investigate the effects of antibody dependent cellular cytotoxicity (ADCC) by the IL-2 conjugate of Example 2 in combination with cetuximab using a co-culture of human PBMCs with calcein-labeled cancer cell lines (CAL27 and A431).

CAL27 Cells.

### Reagents.

Bioassay buffer: 1% ultra low IgG FBS added to phenol-red-free RPMI. Complete assay buffer: 450 μL probenecid added to 45 mL bioassay buffer with final probenecid concentration of 77 μg/mL. Calcein-acetoxymethyl ester (Calcein-AM): 50 μg in 25 μL DMSO. Calcein-AM staining buffer: 10 μL Calcein-AM added to 4 mL complete assay buffer (final Calcein-AM concentration of 5 μg/mL). Triton-X-100 lysis buffer: 20 μL Triton-X-100 added to 4 mL complete assay buffer (final concentration of 0.5%).

### Procedure.

On Day 1, a 6-point, 1 in 5 dilution series (in PBS) of the IL-2 conjugate was prepared. The IL-2 conjugate concentrations were 2, 0.4, 0.08, 0.016, 0.0032, and 0 μg/mL. PBMCs were collected by centrifugation at 200×g for 5 minutes and resuspended in phenol red-free RPMI+10% ultra-low IgG at 20 million cells/mL. Appropriate volumes of these PBMCs were transferred to 6 sections of a multi-well reservoir to which a range of the IL-2 conjugate dilutions was added. PBMCs were mixed well with the IL-2 conjugate by pipetting up and down and 50 mL were transferred into round-bottomed 96 well plates using a multi-channel pipette (final PBMC number per well was 1 million). Six empty wells were reserved for controls to be added the following day. The plates were incubated overnight in a humidified incubator at 37° C. in the presence of 5% carbon dioxide.

On Day 2, CAL27 cells (EGFR-expressing oral epithelial squamous cell carcinoma cell line) were harvested using TrypLE express dissociation buffer and collected by centrifugation at 200×g for 5 minutes. Cells were counted and 5 million cells were resuspended in 4 mL calcein-AM staining buffer and incubated for 30 minutes at 37° C. in the presence of 5% carbon dioxide. Cells were then collected and washed twice in complete assay buffer by centrifugation at 200×g for 5 minutes. Cells were counted and resuspended at 0.4 million cells/mL for a final target cell number of 20,000/well.

Cetuximab antibody (Eli Lilly & Co.) was diluted to a working concentration of 3×(3, 0.3, 0.03, 0.003 μg/mL) for final assay concentrations of 1, 0.1, 0.001, 0.0001 μg/mL. The isotype control (hIgG1, Biolegend) was diluted to 3 μg/mL for a final concentration of 1 μg/mL in complete assay buffer. Equal volumes of stained CAL27 cells at 0.4 million cells/mL were mixed with antibody dilutions or isotype control and incubated for 30 minutes at 4° C. to allow antibody to bind. Following incubation, 100 μL of antibody-CAL27 cell mixture were added to the 96 well plates containing 50 μL of the IL-2 conjugate treated PBMCs from Day 1.

Control wells without PBMCs but with 50 μL calcein-AM stained CAL27 cells treated with complete assay buffer (background signal) or stained CAL27 with 50 μL Triton-X-100 treatment (for maximum signal following cell lysis), both made up to 150 μL final volume with complete assay buffer were prepared in triplicate. The plates were centrifuged for 1 minute at 200×g, and then incubated for 60 minutes at 37° C. in the presence of 5% carbon dioxide. After incubation, the plates were again briefly centrifuged before transferring 90 μL of supernatant into fresh black, clear-bottomed plates, and the fluorescence signal was read on an Envision 2104 plate reader (excitation: 492 nm; emission: 515 nm).

The cytotoxicity was calculated using the following formula:

$$Cytotoxicity ( \% ) = ( A - B ) / ( C - B ) \times 100$$

where A is the fluorescence value for treated cells; B is the background from target cells alone; and C is the maximum release valued obtained from Triton-X-100 treatment.

The data represent the % cytotoxicity of the IL-2 conjugate treated human PBMCs on target cancer cells in the presence of cetuximab. The mean percentage from the technical replicates was converted to a proportion. The analysis was conducted using a two-way generalized linear mixed model (GLMM), with factors for the IL-2 conjugate, cetuximab and their interaction, with random donor effects, treating proportion as a pseudo-binomial variable. It was followed by a post-hoc test (with Dunnett-Hsu adjustment) to compare the IL-2 conjugate treated groups to the control group. Statistical analyses were performed using SAS (1) version 9.4 software. A probability less than 5% (p<0.05) was considered as significant.

### Results

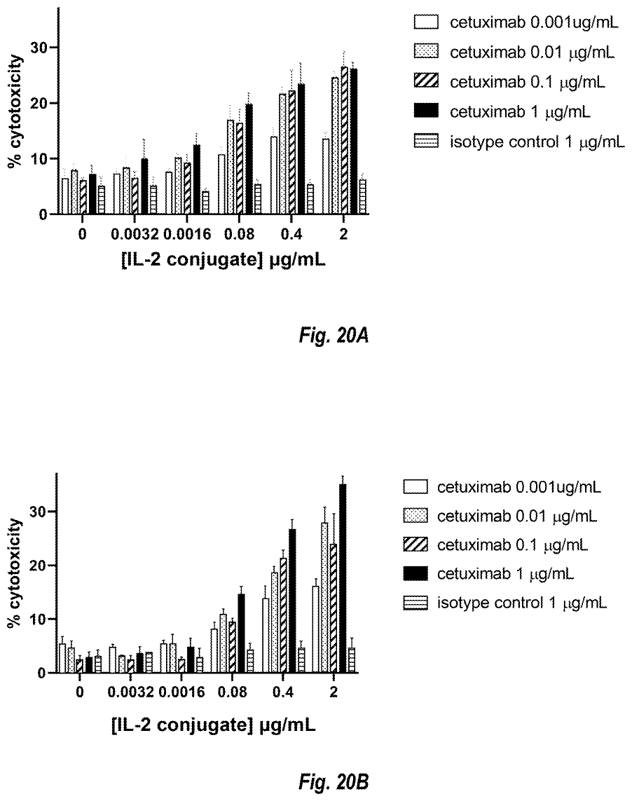

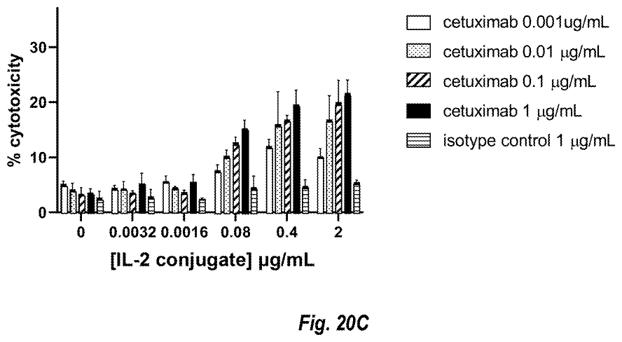

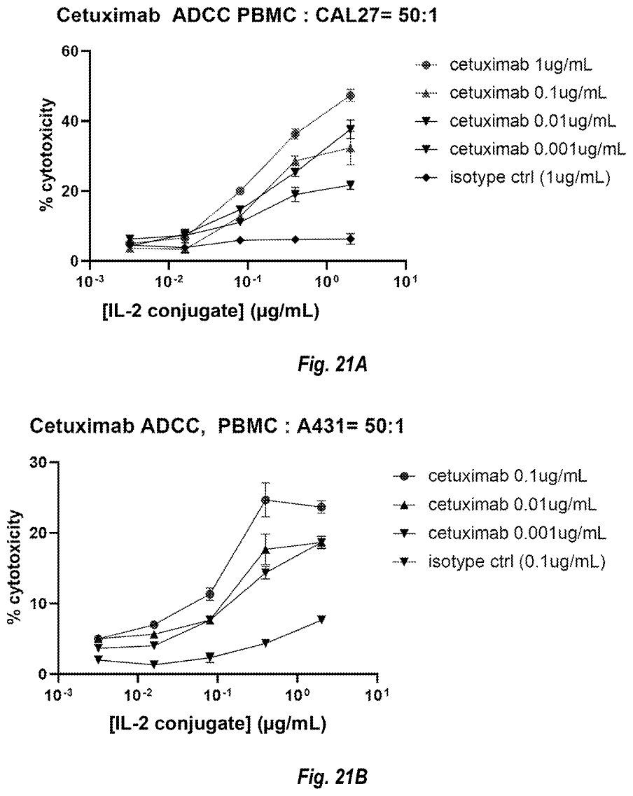

At cetuximab dose levels of 1, 0.1, and 0.01 mg/mL, the IL-2 conjugate enhanced ADCC function of cetuximab against EGFR expressing CAL27 cells (p<0.05) at concentrations of 0.08, 0.4 and 2 mg/mL (FIGS. 20A-C). At a cetuximab dose level of 0.001 mg/mL, the IL-2 conjugate enhanced ADCC function of cetuximab against EGFR expressing CAL27 cells (p<0.05) at concentrations of 0.4 and 2 mg/mL. FIG. 21A further shows the enhanced ADCC function of cetuximab against EGFR expressing CAL27 cells (PBMC to CAL27 ratio 50:1).

The tests of fixed effects from the GLMM model indicate that the factors IL-2 conjugate, cetuximab and their interaction have a significant effect on the cytotoxicity, i.e., the differences between IL-2 conjugate groups vary significantly for the different cetuximab concentrations. The pairwise comparisons indicated a significant difference between the IL-2 conjugate 2 mg/mL group versus the control group (p=0.0001) and between the IL-2 conjugate 0.4 mg/mL group versus the control group (p=0.0001) at a cetuximab concentration of 0.001 mg/mL. The pairwise comparisons also indicated a significant difference between the IL-2 conjugate 2 mg/mL group versus the control group (p<0.0001), between the IL-2 conjugate 0.4 mg/mL group versus the control group (p<0.0001), and between the IL-2 conjugate 0.08 mg/mL group versus the control group (p=0.0003) at a cetuximab concentration of 0.01 mg/mL. In addition, the pairwise comparisons indicated a significant difference between the IL-2 conjugate 2 mg/mL group versus the control group (p<0.0001), between the IL-2 conjugate 0.4 mg/mL group versus the control group (p<0.0001), and between the IL-2 conjugate 0.08 mg/mL group versus the control group (p<0.0001) at a cetuximab concentration of 0.1 mg/mL. Lastly, the pairwise comparisons indicated a significant difference between the IL-2 conjugate 2 mg/mL group versus the control group (p<0.0001), between the IL-2 conjugate 0.4 mg/mL group versus the control group (p<0.0001), and between the IL-2 conjugate 0.08 mg/mL group versus the control group (p<0.0001) at a cetuximab concentration of 1 mg/mL.

The data demonstrate that the IL-2 conjugate enhanced ADCC function of cetuximab against EGFR expressing CAL27 cancer cells. No significant differences were observed using the IL-2 conjugate in combination with the isotype control.

### A431 Cells.

Studies were performed using EGFR expressing A431 cells (epidermoid carcinoma) following the procedure outlined above for CAL27 cells. FIG. 21B shows the enhanced ADCC function of cetuximab against EGFR expressing A431 cells (PBMC to A431 ratio 50:1). The data demonstrate that the IL-2 conjugate enhanced ADCC function of cetuximab against EGFR expressing A431 cancer cells.

### Example 8. ADCC Assay Using an Engineered Cell Line NK-92.CD16 V as Effector Cells

The effect of the IL-2 conjugate of Example 2 on ADCC function of cetuximab was examined using a calcein-acetyoxymethyl (Calcein-AM; Invitrogen) release assay.

### Materials

NK-92.CD16 V (high affinity variant) (Conkwest Inc., San Diego, CA) was used as the effector cell line. The following cell lines were used as target cells: CAL27, A431, DLD-1, and FaDu.

The following reagents were used: cetuximab antibody (Eli Lilly & Co.); human isotype IgG1 antibody (Biolegend); calcein-acetyoxymethyl (Calcein-AM; Invitrogen C3100MP), and probenecid (Invitrogen; P36400). The bioassay medium was phenol red-free RPMI with 1% ultra low IgG fetal bovine serum, supplemented with 1% probenecid for complete assay medium. MyeloCult H5100 (Stemcell Cat #05150) supplemented with IL-2 (100 U/mL) and hydrocortisone (Sigma H6909; 10 mL at 50 μM) was used for the NK-92.CD16 V cell culture.

### Procedure.

IL-2 supplement was withdrawn from the NK-92.CD16 V cell culture, which was then incubated overnight prior to starting the assay. The next day, cells were plated in 96-well round-bottom plates (60,000 cells were plated for a 3:1 ratio of effector to target cells) in the presence of IL-2_P65_[AzK_L1_PEG30kD]-1 at varying concentrations (0.1 μg/mL, 0.01 μg/mL, 0.001 μg/mL, and 0 μg/mL) in phenol red-free RPMI 1640 media supplemented with 1% low IgG FBS for 18 hours at 37° C. in a humidified incubator with 5% CO₂. These cells are used as the effector cells. The following day, human EGFR positive cancer cell lines (A431, DLD-1, FaDu, or CAL27) were labeled with calcein-AM for 30 min (50 μg diluted in 25 μL DMSO to prepare a stock solution, then 10 μL of calcein stock solution was added to 4 mL RPMI 1640 containing 1% low IgG FBS and 1% probenecid for the staining of 5×10⁶ cells) and then washed. Cells were divided into several labeled tubes for incubation with varying concentrations of cetuximab or isotype control. Cetuximab and isotype human IgG1 antibody were added at 3×concentrations (for final assay concentrations from 10 μg/mL to 1 μg/mL), and the labeled target cells and antibody were mixed and allowed to sit for 30 min to allow opsonization. After this incubation, target cells (20,000) and antibody were added on top of NK-92.CD16 V cells in 100 μL. The plate was centrifuged briefly for 1 minute at 1100 rpm before incubating at 37° C. and 5% CO₂ for 1 hour.

Following incubation, the plates were again briefly centrifuged as before, and 90 μL of supernatant was transferred from each well to black plates with clear bottom without disturbing the cells. The fluorescence signal was read using Envision 2104 (excitation: 492 nm; emission: 515 nm).

For maximal release, the cells were lysed with 2% Triton X-100. The fluorescence value of the culture medium background was subtracted from that of the experimental release (A), the target cell spontaneous release (B), and the target cell maximal release (C).

The cytotoxicity and ADCC percentages for each plate (in duplicate) were calculated using the following formulas:

$$Cytotoxicity ( \% ) = ( A - B ) / ( C - B ) \times 100$$

ADCC (%)=Cytotoxicity (%, with antibody)−Cytotoxicity (%, without antibody)

For each experiment, measurements were conducted in triplicate using three replicate wells. Each experiment is repeated at least 3 times. The half-maximal effective concentration (EC50) values are calculated by fitting the data points to a 4-parameter equation using GraphPad Prism 5 (GraphPad Software, Inc., San Diego, CA).

### RESULTS

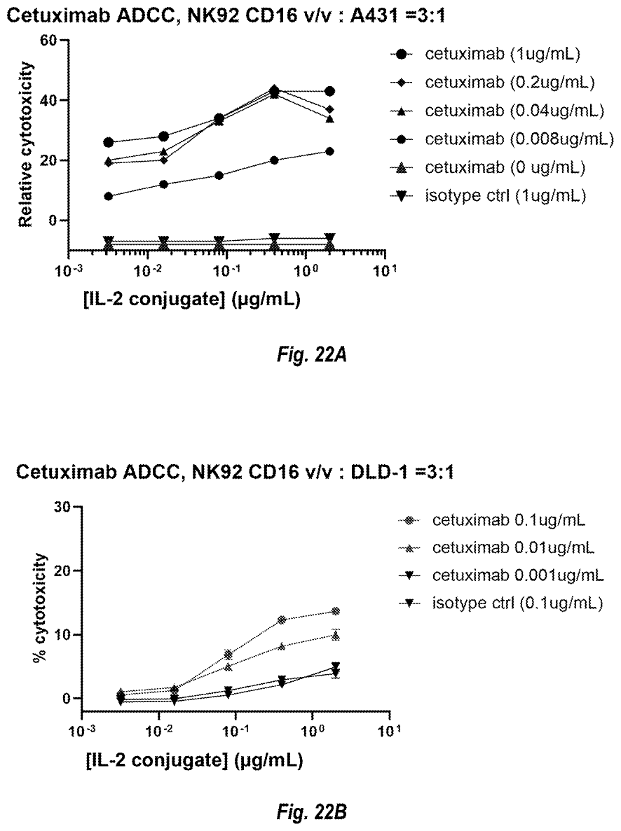

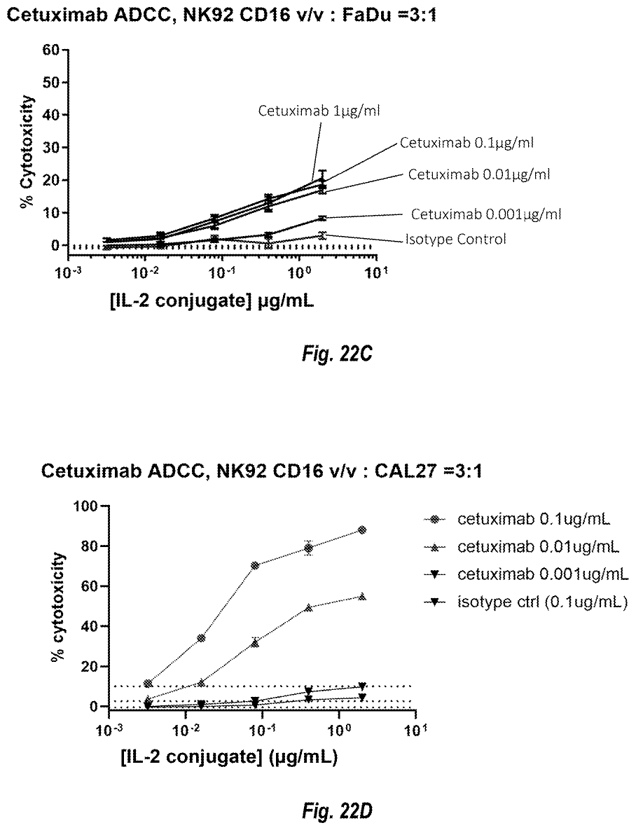

Cytotoxicity data using the NK92 cell line ADCC assay is shown in FIGS. 22A-D for EGFR expressing A431 (epidermoid carcinoma) (NK92 to A431 ratio 3:1), DLD-1 (adenocarcinoma, colorectal) (NK92 to DLD-1 ratio 3:1), FaDu (epithelial squamous cell carcinoma) (NK92 to FaDu ratio 3:1), and CAL27 (epithelial squamous cell carcinoma) (NK92 to CAL27 ratio 3:1) cells, respectively. The data demonstrate that the IL-2 conjugate enhanced ADCC function of cetuximab against EGFR expressing cancer cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating head and neck squamous cell carcinoma (HNSCC) in a subject in need thereof, comprising administering to the subject a combination therapy comprising (a) an IL-2 conjugate and (b) pembrolizumab, wherein:
the subject has recurrent and/or metastatic HNSCC; and
the IL-2 conjugate comprises the amino acid sequence of SEQ ID NO: 1 wherein the amino acid at position P64 is replaced by the structure of Formula (I): 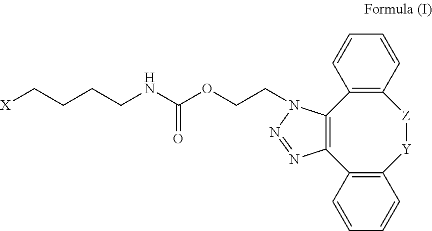
wherein:
Z is CH₂ and Y is 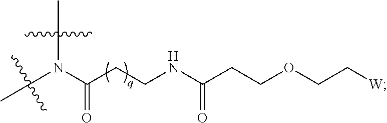
Y is CH₂ and Z is 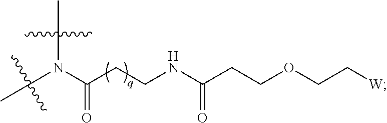
Z is CH₂ and Y is 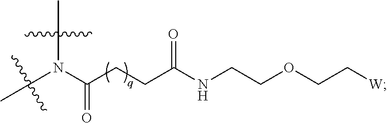
or
Y is CH₂ and Z is 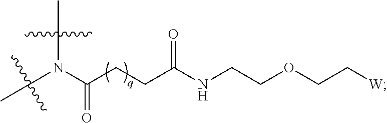
W is a PEG group having an average molecular weight of about 25 kDa-35 kDa;
q is 1, 2, or 3;
X is an L-amino acid having the structure: 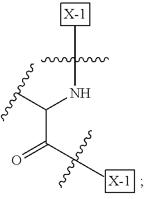
X−1 indicates the point of attachment to the preceding amino acid residue; and
X+1 indicates the point of attachment to the following amino acid residue.

2. A method of treating head and neck squamous cell carcinoma (HNSCC) in a subject in need thereof, comprising:
selecting a subject having HNSCC, wherein the subject is selected at least in part on the basis of the subject having recurrent and/or metastatic HNSCC; and
administering to the subject a combination therapy comprising (a) an IL-2 conjugate, and (b) pembrolizumab, wherein:
the IL-2 conjugate comprises the amino acid sequence of SEQ ID NO: 1 wherein the amino acid at position P64 is replaced by the structure of Formula (I): 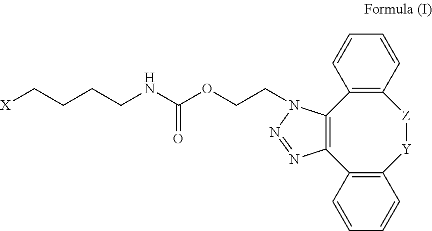
wherein:
Z is CH₂ and Y is 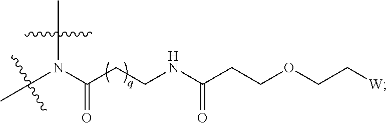
Y is CH₂ and Z is 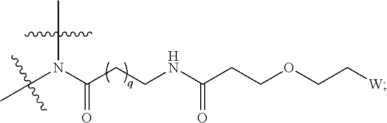
Z is CH₂ and Y is 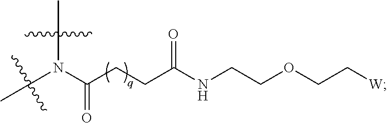
or
Y is CH₂ and Z is 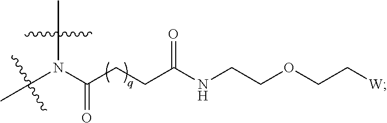
W is a PEG group having an average molecular weight of about 25 kDa-35 kDa;
q is 1, 2, or 3;
X is an L-amino acid having the structure: 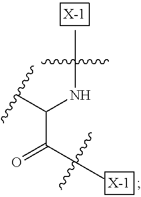
X−1 indicates the point of attachment to the preceding amino acid residue; and
X+1 indicates the point of attachment to the following amino acid residue.

3. (canceled)

4. (canceled)

5. A method of treating head and neck squamous cell carcinoma (HNSCC) in a subject in need thereof, comprising administering to the subject a combination therapy comprising (a) an IL-2 conjugate, (b) pembrolizumab, and (c) cetuximab, wherein:
the IL-2 conjugate comprises the amino acid sequence of SEQ ID NO: 1 wherein the amino acid at position P64 is replaced by the structure of Formula (I): 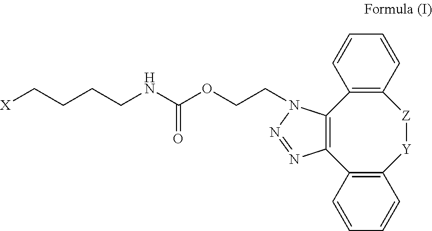
wherein:
Z is CH₂ and Y is 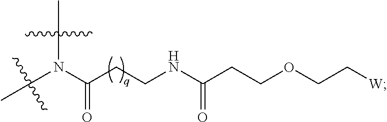
Y is CH₂ and Z is 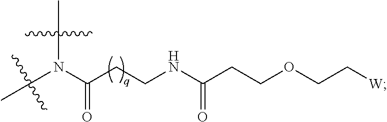
Z is CH₂ and Y is 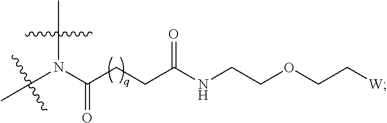
or
Y is CH₂ and Z is 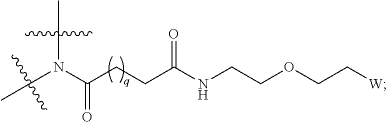
W is a PEG group having an average molecular weight of about 25 kDa-35 kDa;
q is 1, 2, or 3;
X is an L-amino acid having the structure: 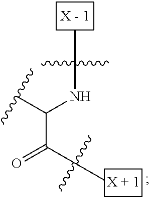 [SMILES: *NC(*)C(*)=O]
X−1 indicates the point of attachment to the preceding amino acid residue; and
X+1 indicates the point of attachment to the following amino acid residue.

6. A method of treating head and neck squamous cell carcinoma (HNSCC) in a subject in need thereof, comprising administering to the subject a combination therapy comprising (a) an IL-2 conjugate, (b) pembrolizumab, and (c) an anti-transforming growth factor beta (TGFβ) antibody, wherein:
the IL-2 conjugate comprises the amino acid sequence of SEQ ID NO: 1 wherein the amino acid at position P64 is replaced by the structure of Formula (I): 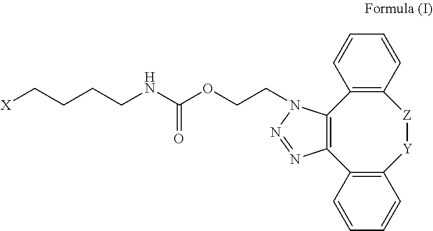
wherein:
Z is CH₂ and Y is 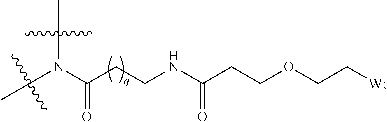
Y is CH₂ and Z is 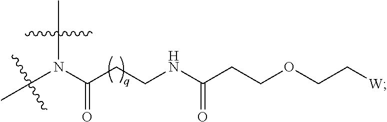
Z is CH₂ and Y is 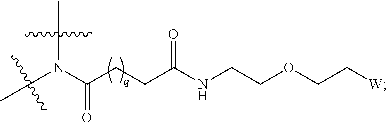
or
Y is CH₂ and Z is 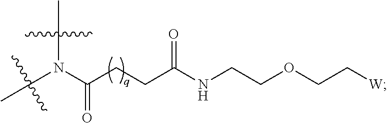
W is a PEG group having an average molecular weight of about 25 kDa-35 kDa;
q is 1, 2, or 3;
X is an L-amino acid having the structure: 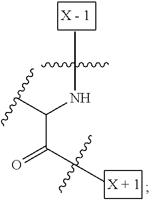 [SMILES: *NC(*)C(*)=O]
X−1 indicates the point of attachment to the preceding amino acid residue; and
X+1 indicates the point of attachment to the following amino acid residue.

7. The method of claim 1, wherein the subject has a PD-L1 combined positive score (CPS) greater than or equal to 1.

8. The method of claim 1, wherein the subject is treatment-naïve for recurrent and/or metastatic HNSCC.

9. A method of treating head and neck squamous cell carcinoma (HNSCC) in a subject in need thereof, comprising administering to the subject a combination comprising (a) an IL-2 conjugate and (b) pembrolizumab, wherein:
the HNSCC is recurrent and/or metastatic HNSCC; and
the IL-2 conjugate comprises the amino acid sequence of SEQ ID NO: 1 wherein the amino acid at position P64 is replaced by the structure of Formula (I): 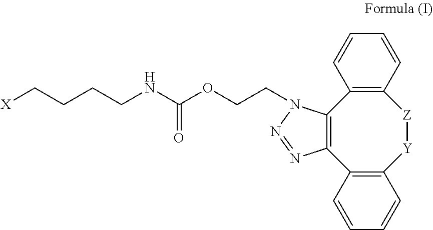
wherein:
Z is CH₂ and Y is 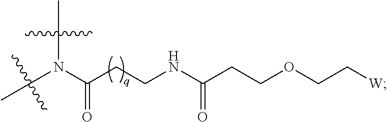
Y is CH₂ and Z is 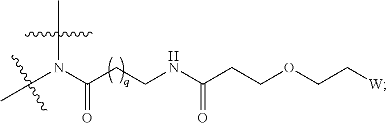
Z is CH₂ and Y is 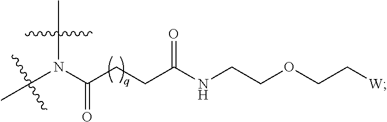
or
Y is CH₂ and Z is 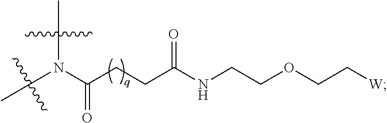
W is a PEG group having an average molecular weight of about 25 kDa-35 kDa;
q is 1, 2, or 3;
X is an L-amino acid having the structure: 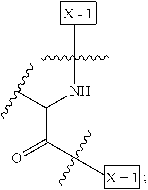 [SMILES: *NC(*)C(*)=O]
X−1 indicates the point of attachment to the preceding amino acid residue; and
X+1 indicates the point of attachment to the following amino acid residue.

10. A method of treating head and neck squamous cell carcinoma (HNSCC) in a subject in need thereof, comprising:
selecting a subject having HNSCC, wherein the subject is selected at least in part on the basis of the subject having recurrent and/or metastatic HNSCC; and
administering to the subject a combination comprising (a) an IL-2 conjugate, and (b) pembrolizumab, wherein:
the IL-2 conjugate comprises the amino acid sequence of SEQ ID NO: 1 wherein the amino acid at position P64 is replaced by the structure of Formula (I): 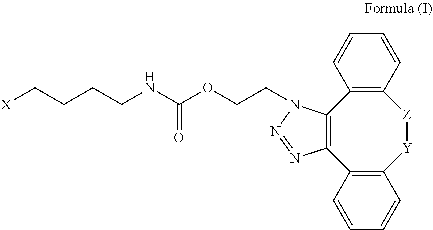
wherein:
Z is CH₂ and Y is 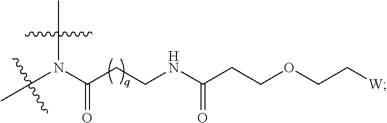
Y is CH₂ and Z is 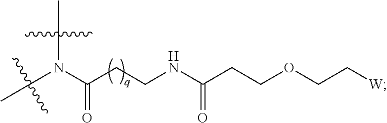
Z is CH₂ and Y is 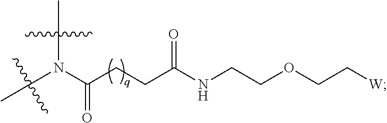
or
Y is CH₂ and Z is 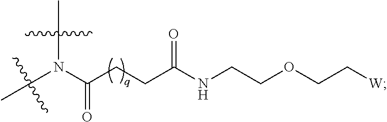
W is a PEG group having an average molecular weight of about 25 kDa-35 kDa;
q is 1, 2, or 3;
X is an L-amino acid having the structure: 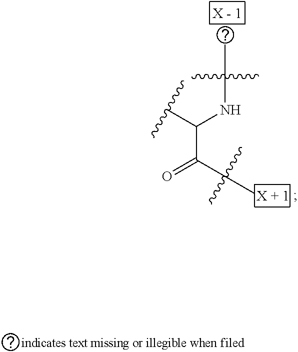 [SMILES: *NC(*)C(*)=O]
X−1 indicates the point of attachment to the preceding amino acid residue; and
X+1 indicates the point of attachment to the following amino acid residue.

11. The method of claim 9, wherein the subject was previously treated with a PD-1/PD-L1-based regimen.

12. The method of claim 3, wherein the subject was not previously treated with cetuximab.

13. The method of claim 9, wherein the subject has platinum-refractory HNSCC.

14. The method of claim 9, wherein the subject was previously treated for HNSCC and the previous treatment for HNSCC comprised failure of no more than two regimens.

15. The method of claim 9, wherein the subject has platinum-refractory HNSCC and the subject's previous treatment for HNSCC comprised failure of one regimen.

16. The method of claim 9, wherein the subject has platinum-refractory HNSCC and the subject's previous treatment for HNSCC comprised failure of two regimens.

17. The method of claim 1, comprising administering to the subject about 8 μg/kg to 32 μg/kg of the IL-2 conjugate.

18-21. (canceled)

22. The method of claim 1, wherein in the IL-2 conjugate the PEG group has an average molecular weight of about 30 kDa.

23. The method of claim 1, wherein in the IL-2 conjugate Z is CH₂ and Y is

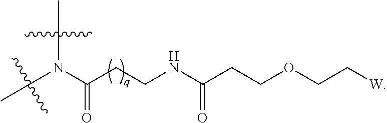

24. The method of claim 1, wherein in the IL-2 conjugate Y is CH₂ and Z is

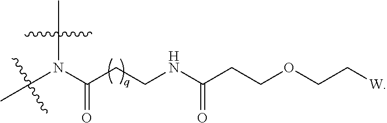

25. The method of claim 1, wherein in the IL-2 conjugate Z is CH₂ and Y is

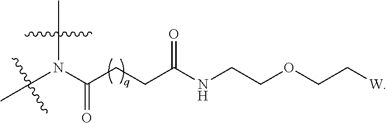

26. The method of claim 1, wherein in the IL-2 conjugate Y is CH₂ and Z is

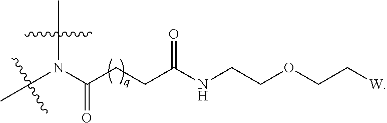

27. The method of claim 1, wherein the structure of Formula (I) has the structure of Formula (IV) or Formula (V), or is a mixture of Formula (IV) and Formula (V): 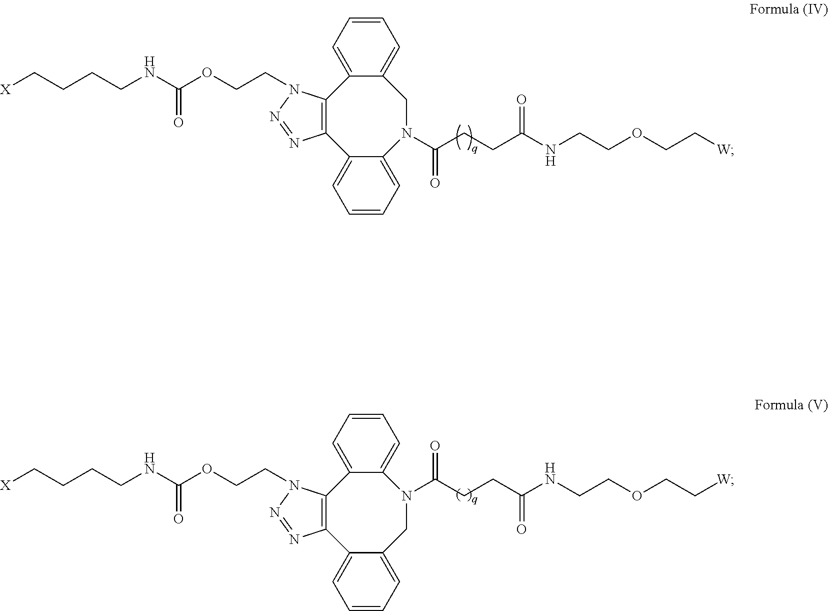
wherein:
W is a PEG group having an average molecular weight of about 25 kDa-35 kDa;
q is 1, 2, or 3;
X is an L-amino acid having the structure: 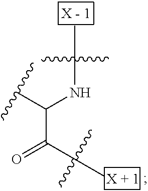 [SMILES: *NC(*)C(*)=O]
X−1 indicates the point of attachment to the preceding amino acid residue; and
X+1 indicates the point of attachment to the following amino acid residue.

28. The method of claim 1, wherein the structure of Formula (I) has the structure of Formula (XII) or Formula (XIII), or is a mixture of Formula (XII) and Formula (XIII): 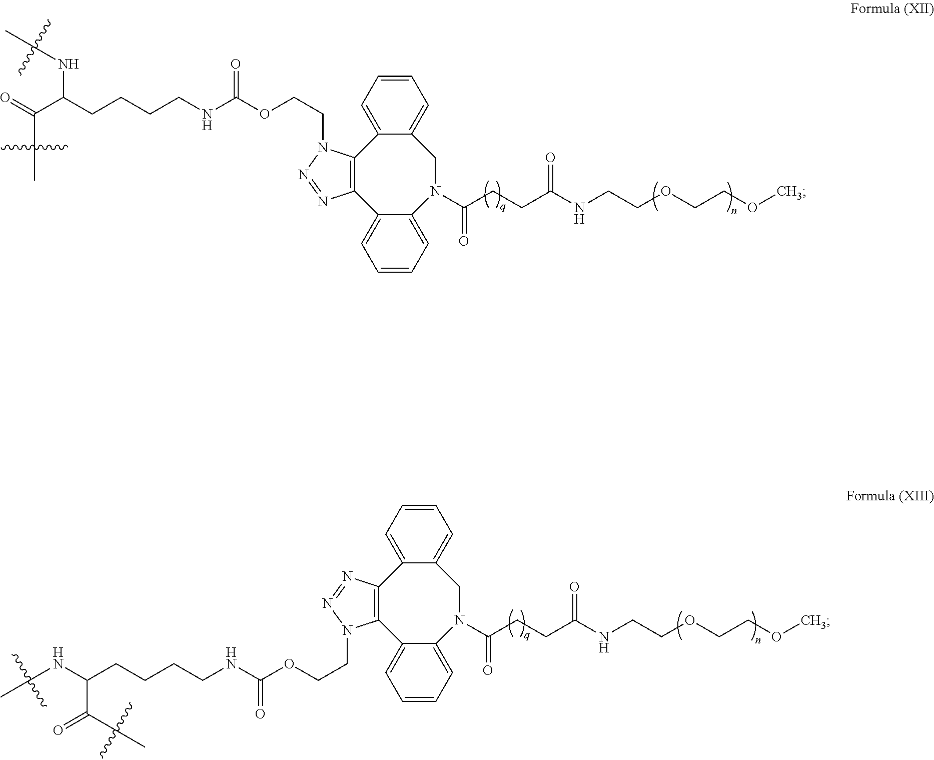
wherein:
n is an integer such that —(OCH₂CH₂)˜—OCH₃ has a molecular weight of about 30 kDa;
q is 1, 2, or 3; and
the wavy lines indicate covalent bonds to amino acid residues within SEQ ID NO: 1 that are not replaced.

29. The method of claim 1, wherein q is 1.

30-37. (canceled)

38. The method of claim 1, wherein the IL-2 conjugate is a pharmaceutically acceptable salt, solvate, or hydrate.

39. The method of claim 1, wherein pembrolizumab is administered at a dose of about 200 mg every 3 weeks.

40. The method of claim 1, wherein pembrolizumab is administered at a dose of about 400 mg every 6 weeks.

41. (canceled)

42. The method of claim 1, wherein the IL-2 conjugate and pembrolizumab are administered separately.

43. The method of claim 42, wherein the IL-2 conjugate and pembrolizumab are administered sequentially.

44. The method of claim 43, wherein the IL-2 conjugate is administered after pembrolizumab.

45. The method of claim 43, wherein pembrolizumab is administered after the IL-2 conjugate.

46. (canceled)

47. The method of claim 3, wherein cetuximab is administered after pembrolizumab.

48. The method of claim 3, wherein cetuximab is administered before the IL-2 conjugate.

49. (canceled)

50. (canceled)

51. The method of claim 4, wherein the anti-TGFβ antibody is administered after the IL-2 conjugate.

52-72. (canceled)


SEQUENCE LISTING (40 sequences)

SEQ ID NO: 1 - protein, 132 aa, synthetic construct
  REGION 1..132: note = IL-2 conjugate
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 132 aa, synthetic construct
  REGION 1..132: note = IL-2 conjugate
  MOD_RES 64: note = [AzK_L1_PEG30kD], which is N6-((2-azidoethoxy)-carbonyl)-L-lysine stably-conjugated to PEG. The PEG group has an average molecular weight of about 25-35 kiloDaltons (e.g., about 30 kDa), capped with a methoxy group.
PTSSSTKKTQLQLEHLLLDLQMILNGINNYKNPKLTRMLTFKFYMPKKATELKHLQCLEEELKLLEEVLNLAQSKNFHLRPRDLISNINVIVLELKGSETTFMCEYADETATIVEFLNRWITFSQSIISTLT

SEQ ID NO: 3 - protein, 215 aa, synthetic construct
  REGION 1..215: note = light chain sequence of fresolimumab (GC1008)
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 106 aa, synthetic construct
  REGION 1..106: note = VL sequence of fresolimumab (GC1008)
TVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEI

SEQ ID NO: 5 - protein, 218 aa, synthetic construct
  REGION 1..218: note = heavy chain sequence of fresolimumab (GC1008)
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 6 - protein, 116 aa, synthetic construct
  REGION 1..116: note = VH sequence of fresolimumab (GC1008)
LVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVS

SEQ ID NO: 7 - protein, 215 aa, synthetic construct; identical to SEQ ID NO: 3
  REGION 1..215: note = anti-TGFbeta light chain

SEQ ID NO: 8 - protein, 447 aa, synthetic construct
  REGION 1..447: note = anti-TGFbeta heavy chain
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 104 aa, synthetic construct
  REGION 1..104: note = VL sequence of the anti-TGFbeta antibody
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTR

SEQ ID NO: 10 - protein, 12 aa, synthetic construct
  REGION 1..12: note = LCDR-1 sequence of the anti-TGFbeta antibody
RASQSLGSSYLA

SEQ ID NO: 11 - protein, 7 aa, synthetic construct
  REGION 1..7: note = LCDR-2 sequence of the anti-TGFbeta antibody
GASSRAP

SEQ ID NO: 12 - protein, 9 aa, synthetic construct
  REGION 1..9: note = LCDR-3 sequence of the anti-TGFbeta antibody
QQYADSPIT

SEQ ID NO: 13 - protein, 120 aa, synthetic construct
  REGION 1..120: note = VH sequence of the anti-TGFbeta antibody
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 14 - protein, 5 aa, synthetic construct
  REGION 1..5: note = HCDR-1 sequence of the anti-TGFbeta antibody
SNVIS

SEQ ID NO: 15 - protein, 17 aa, synthetic construct
  REGION 1..17: note = HCDR-2 sequence of the anti-TGFbeta antibody
GVIPIVDIANYAQRFKG

SEQ ID NO: 16 - protein, 11 aa, synthetic construct
  REGION 1..11: note = HCDR-3 sequence of the anti-TGFbeta antibody
TLGLVLDAMDY

SEQ ID NO: 17 - protein, 15 aa, synthetic construct
  REGION 1..15: note = Pembrolizumab Light Chain CDR1
RASKGVSTSGYSYLH

SEQ ID NO: 18 - protein, 7 aa, synthetic construct
  REGION 1..7: note = Pembrolizumab Light Chain CDR2
LASYLES

SEQ ID NO: 19 - protein, 9 aa, synthetic construct
  REGION 1..9: note = Pembrolizumab Light Chain CDR3
QHSRDLPLT

SEQ ID NO: 20 - protein, 111 aa, synthetic construct [sequence omitted]

SEQ ID NO: 21 - protein, 218 aa, synthetic construct
  REGION 1..218: note = Pembrolizumab Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 5 aa, synthetic construct
  REGION 1..5: note = Pembrolizumab Heavy Chain CDR1
NYYMY

SEQ ID NO: 23 - protein, 17 aa, synthetic construct
  REGION 1..17: note = Pembrolizumab Heavy Chain CDR2
GINPSNGGTNFNEKFKN

SEQ ID NO: 24 - protein, 11 aa, synthetic construct
  REGION 1..11: note = Pembrolizumab Heavy Chain CDR3
RDYRFDMGFDY

SEQ ID NO: 25 - protein, 120 aa, synthetic construct [sequence omitted]

SEQ ID NO: 26 - protein, 447 aa, synthetic construct
  REGION 1..447: note = Pembrolizumab Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 15 aa, synthetic construct
  REGION 1..15: note = hPD-1.08A CDRL1
RASKSVSTSGFSWLH

SEQ ID NO: 28 - protein, 7 aa, synthetic construct
  REGION 1..7: note = hPD-1.08A CDRL2
LASNLES

SEQ ID NO: 29 - protein, 9 aa, synthetic construct
  REGION 1..9: note = hPD-1.08A CDRL3
QHSWQLPLT

SEQ ID NO: 30 - protein, 5 aa, synthetic construct
  REGION 1..5: note = hPD-1.08A CDRH1
SYYLY

SEQ ID NO: 31 - protein, 16 aa, synthetic construct
  REGION 1..16: note = hPD-1.08A CDRH2
GVNPSNGGTNFSEKFK

SEQ ID NO: 32 - protein, 11 aa, synthetic construct
  REGION 1..11: note = hPD-1.08A CDRH3
RDSNYDGGFDY

SEQ ID NO: 33 - protein, 122 aa, synthetic construct
  REGION 1..122: note = Mature h109A heavy chain variable region sequence
QVQLQQPGAELVKPGTSVKLSCKASGYTFTNYYMYWVKQRPGQGLEWIGGINPSNGGTNFNGKFKNKATLTVDSSSSTTYMQLSSLTSEDSAVYYCTRRDYRFDMGFDYWGQGTTLTVSSAK

SEQ ID NO: 34 - protein, 130 aa, synthetic construct
  REGION 1..130: note = Mature K09A light chain variable sequence
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 35 - protein, 130 aa, synthetic construct
  REGION 1..130: note = Mature K09A light chain variable sequence
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 36 - protein, 130 aa, synthetic construct
  REGION 1..130: note = Mature K09A light chain variable sequence
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 37 - protein, 466 aa, synthetic construct
  REGION 1..466: note = Mature 409 heavy chain sequence
MAVLGLLFCLVTFPSCVLSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 237 aa, synthetic construct
  REGION 1..237: note = Mature K09A light chain sequence
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 237 aa, synthetic construct
  REGION 1..237: note = Mature K09A light chain sequence
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 237 aa, synthetic construct
  REGION 1..237: note = Mature K09A light chain sequence
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC